(19) United States
(12) Patent Application Publication
(10) Pub. No.: US 2014/0243544 A1
(43) Pub. Date: Aug. 28, 2014

(54) METHOD OF MAKING 1-(ACYLOXY)-ALKYL CARBAMATE COMPOUNDS

(71) Applicant: XENOPORT, INC., SANTA CLARA, CA (US)

(72) Inventors: Huan Wang, King of Prussia, PA (US); Peng Liu, King of Prussia, PA (US); Qunying Dai, King of Prussia, PA (US); Hao Yin, Vienna, VA (US); Stephen P. Raillard, Mountain View, CA (US)

(73) Assignee: XENOPORT, INC., SANTA CLARA, CA (US)

(21) Appl. No.: 14/189,555

(22) Filed: Feb. 25, 2014

Related U.S. Application Data
(60) Provisional application No. 61/769,554, filed on Feb. 26, 2013.

(51) Int. Cl. C07C 69/96; C07C 255/57; C07C 269/00

(52) U.S. Cl. CPC C07C 69/96; C07C 269/00; C07C 255/57
USPC 558/273; 560/129

(57) ABSTRACT

Methods of preparing carbamate prodrugs of amine-containing drugs are provided. Carbonates useful in the synthesis of the carbamate prodrugs are also provided.

### RELATED APPLICATIONS

This application claims the benefit of the filing date of U.S. Provisional Application No. 61/769,554, filed Feb. 26, 2013, entitled METHOD OF MAKING 1-(ACYLOXY)-ALKYL CARBAMATE COMPOUNDS, the contents of which are herein incorporated by reference.

### FIELD

Provided herein are novel methods useful for preparation of 1-(acyloxy)-alkyl carbamate prodrugs of amino containing drug compounds. Also provided are compositions of novel compounds useful for the preparation of the carbamate prodrugs.

### BACKGROUND

Gabapentin ([1-(aminomethyl)cyclohexyl]acetic acid) is an FDA approved drug that is marketed for the treatment of post-herpetic neuralgia and epilepsy. Gabapentin suffers from poor oral bioavailability, which is primarily due to the fact that it is absorbed by a saturable active transport mechanism in the small intestine. Gabapentin also has a very short half life in vivo, and to maintain therapeutic levels in the body, frequent dosing is required.

The oral bioavailability of certain drugs can be improved by conversion to prodrugs. Certain prodrugs are derivatives of the parent drug in which a functional group is “masked” by a promoiety. Following administration to a patient, the prodrug is metabolised to release the parent drug.

The acyloxyalkoxylcarbonyl functionality is an example of a promoiety that has been used to functionalize amino containing drugs such as gabapentin. 1-{[(α-Isobutanoyloxyethoxy)carbonyl]aminomethyl}-1-cyclohexane acetic acid is a 1-(acyloxy)-alkyl carbamate prodrug of gabapentin that has utility in the treatment of epilepsy (WO 02/100347), pain (WO 02/100347), particularly neuropathic pain (including post-herpetic neuralgia and diabetic peripheral neuropathy) or pain associated with irritable bowel syndrome, anxiety (WO 02/100347), particularly general anxiety disorder, alcohol dependency or ethanol withdrawal syndrome (WO 02/100347), restless legs syndrome (WO 2005/027850), migraine prophylaxis (WO 2008/073257), fibromyalgia (WO 2008/073257), hot flashes (WO 2004/089289), particularly hot flashes associated with the menopause and essential tremor (Patent Application claiming priority from U.S. Provisional Application No. 61/158,065).

Methods of preparing 1-(acyloxy)-alkyl carbamate prodrugs are disclosed in WO 02/100347, WO 03/077902, WO 03/104184, WO 2005/010011 and WO 2005/066122 (all assigned to Xenoport, Inc.), WO 2010/017504 (assigned to Xenoport, Inc. and Glaxo Group Limited), and U.S. Pat. No. 4,760,057, U.S. Pat. No. 4,916,230 and U.S. Pat. No. 5,684,018 (all assigned to Merck & Co. Ltd.).

### SUMMARY

There is still a clear unmet need for improved methods for preparation of the carbamate prodrugs. The compounds, compositions, and methods of preparation described herein are directed toward this end.

Provided herein are novel methods of preparation of 1-(acyloxy)-alkyl carbamate prodrugs of amino containing drug molecules or drug compounds.

In certain aspects, provided herein are novel methods of preparation of 1-(acyloxy)-alkyl carbamate prodrugs of gabapentin and related compounds.

In certain aspect, the present disclosure provides a method of making a compound of formula (I), or a stereoisomer thereof, a diastereomer thereof, or a salt of any one of foregoing, comprising:

(A) reacting a compound of formula (II), or a stereoisomer or a salt thereof, with R¹CO₂H to form a compound of formula (III);

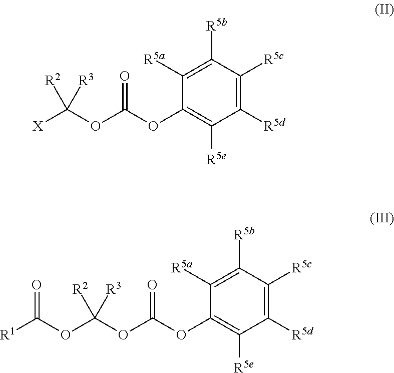

and

(B) reacting the compound of formula (III), or a stereoisomer or a salt thereof, with HNR^{4a}R^{4b} to form the compound of formula (I)

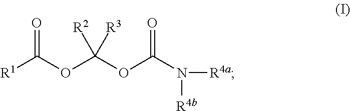

wherein:

each of R¹ and R² is independently C₁₋₄ alkyl;

R³ is H or C₁₋₄ alkyl;

HNR^{4a}R^{4b} is a drug molecule having an amino moiety;

R^{4a} and R^{4a} are groups of the drug molecule attached to the amino moiety;

each of R^{5a}, R^{5b}, R^{5c}, R^{5d}, and R^{5e} is independently selected from H, halo, C₁₋₄ alkyl, halo C₁₋₄ alkyl, phenyl, —C(O)O—C₁₋₄ alkyl, —C(O)—C₁₋₄ alkyl, —S(O)—C₁₋₄ alkyl, CN, —C(O)—NR^{6a}R^{6b}, substituted or unsubstituted C₁₋₄ alkoxy, and substituted or unsubstituted phenoxy;

each of R^{6a} and R^{6b} is independently H, or C₁₋₄ alkyl; or R^{6a} and R^{6b} together with N they are attached to form heterocycle;

provided that at least one of R^{5a}, R^{5b}, R^{5c}, R^{5d}, and R^{5e} is other than H; or any two adjacent R^{5a}, R^{5b}, R^{5c}, R^{5d}, and R^{5e} are joined together to form a carbocycle or heterocycle; and

X is a leaving group.

In some embodiments, R^{4a} is H; and R^{4b} is selected from:

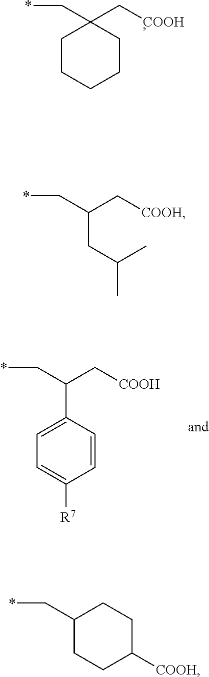

the * indicates the attachment point, and R⁷ is Cl or F.

In another aspect, the present disclosure provides a compound according to formula (III):

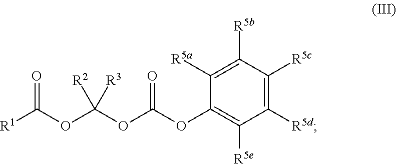

or stereoisomer thereof; or a salt of any one of foregoing;

wherein each of R¹ and R² is independently C₁₋₄ alkyl;

R³ is H or C₁₋₄ alkyl;

each of R^{5a}, R^{5b}, R^{5c}, R^{5d}, or R^{5e} is independently selected from a group consisting of H, halo, C₁₋₄ alkyl, halo C₁₋₄ alkyl, phenyl, —C(O)O—C₁₋₄ alkyl, —C(O)—C₁₋₄ alkyl, —S(O)—C₁₋₄ alkyl, CN, —C(O)—NR^{6a}R^{6b}, substituted or unsubstituted C₁₋₄ alkoxy, and substituted or unsubstituted phenoxy;

each of R^{6a} and R^{6b} is independently H, or C₁₋₄ alkyl; or R^{6a} and R^{6b} together with N they are attached to form heterocycle;

provided that at least one of R^{5a}, R^{5b}, R^{5c}, R^{5d}, and R^{5e} is other than H; or

any two of R^{5a}, R^{5b}, R^{5c}, R^{5d}, and R^{5e} form O—CH₂—O—, or O—CH₂—CH₂—O—.

In another specific aspect, provided herein are compositions comprising compounds according to formula (III).

Other objects and advantages will become apparent to those skilled in the art from a consideration of the ensuing detailed description.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGrawHill, N.Y., 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

When a range of values is listed, it is intended to encompass each value and sub range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl. Similarly, “C₁₋₄ alkyl” is intended to encompass a linear or branched saturated hydrocarbon group containing from 1 to 4 carbon atoms. “C₁₋₄ alkyl” thus encompasses methyl, ethyl, n-propyl, isopropyl, n-butyl, isobutyl, sec-butyl and tert butyl.

The following terms are intended to have the meanings presented therewith below and are useful in understanding the description and intended scope of the present disclosure. When describing the subject matter of the present disclosure, which may include compounds, pharmaceutical compositions containing such compounds and methods of using such compounds and compositions, the following terms, if present, have the following meanings unless otherwise indicated. It should also be understood that when described herein any of the moieties defined below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tertbutyl (C₄), secbutyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl.

The term ‘halo C_{x-y} alkyl’ as used herein refers to a C_{x-y} alkyl group as defined herein wherein at least one hydrogen atom is replaced with halogen. Examples of halo C₁₋₃ alkyl groups include fluoroethyl, trifluoromethyl or trifluoroethyl and the like.

“Acyl” refers to a radical —C(O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), C(O)—C₁-C₈ alkyl, C(O)—(CH₂)ₜ(C₆-C₁₀ aryl), —C(O)—(CH₂)ₜ(5-10 membered heteroaryl), C(O)—(CH₂)ₜ(C₃-C₁₀ cycloalkyl), and —C(O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄haloalkyl, unsubstituted C₁-C₄ hydroxyalkyl, or unsubstituted C₁-C₄ haloalkoxy or hydroxy.

The term ‘halo’ as used herein refers to fluoro, chloro, bromo or iodo.

“Compounds,” “compounds of the present disclosure”, and equivalent expressions, are meant to embrace the compounds as hereinbefore described, in particular compounds according to any of the Formula herein recited and/or described, which expression includes the prodrugs, the pharmaceutically acceptable salts, and the solvates, e.g., hydrates, where the context so permits. Similarly, reference to intermediates, whether or not they themselves are claimed, is meant to embrace their salts, and solvates, where the context so permits.

These and other examples of substituents are described in more detail in the Detailed Description, Examples, and claims. The present disclosure is not intended to be limited in any manner by the above listing of examples of substituents.

### OTHER DEFINITIONS

“Prodrugs” refers to compounds, including derivatives of the compounds of the present disclosure, which have cleavable groups and become by solvolysis or under physiological conditions the compounds of the present disclosure that are pharmaceutically active in vivo. Such examples include, but are not limited to, choline ester derivatives and the like, N-alkylmorpholine esters and the like. Other derivatives of the compounds of this present disclosure have activity in both their acid and acid derivative forms, and in some embodiments the acid sensitive form offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this disclosure are particular prodrugs. In some cases it is desirable to prepare double ester type prodrugs such as (acyloxy)alkyl esters or ((alkoxycarbonyl)oxy)alkylesters. Particularly the C₁ to C₈ alkyl, C₂-C₈ alkenyl, C₂-C₈ alkynyl, aryl, C₇-C₁₂ substituted aryl, and C₇-C₁₂ arylalkyl esters of the compounds of the present disclosure.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the present disclosure may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example when it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 80% by weight R-compound and at most about 20% by weight S-compound, at least about 90% by weight R-compound and at most about 10% by weight S-compound, at least about 95% by weight R-compound and at most about 5% by weight S-compound, at least about 99% by weight R-compound and at most about 1% by weight S-compound, at least about 99.9% by weight R-compound or at most about 0.1% by weight S-compound. In certain embodiments, the weights are based upon total weight of compound.

As used herein and unless otherwise indicated, the term “enantiomerically pure 5-compound” or “S-compound” refers to at least about 80% by weight S-compound and at most about 20% by weight R-compound, at least about 90% by weight S-compound and at most about 10% by weight R-compound, at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this disclosure may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Reference to “about” a value or parameter herein includes (and describes) variations that are directed to that value or parameter per se. For example, a description referring to “about X” includes the description of “X”.

As used herein and in the appended claims, the singular forms “a,” “or,” and “the” include plural referents unless the context clearly dictates otherwise. It is understood that aspects and variations of the subject matter described and disclosed herein include “consisting” and/or “consisting essentially of” aspects and variations.

Unless defined otherwise or clearly indicated by context, all technical and scientific terms and abbreviations used herein have the same meaning as commonly understood by one of ordinary skill in the art to which this disclosure belongs.

### DETAILED DESCRIPTION

Provided herein are novel methods of preparation of 1-(acyloxy)-alkyl carbamate prodrugs of amino containing drug molecules.

In certain aspects, provided herein are novel methods of preparation of 1-(acyloxy)-alkyl carbamate prodrugs of gabapentin and related compounds.

In certain aspects, the present disclosure provides a method of making a compound of formula (I), or a stereoisomer thereof, a diastereomer thereof, or a salt of any one of foregoing, comprising:

(A) reacting a compound of formula (II), or a stereoisomer or a salt thereof, with R¹CO₂H to form a compound of formula (III);

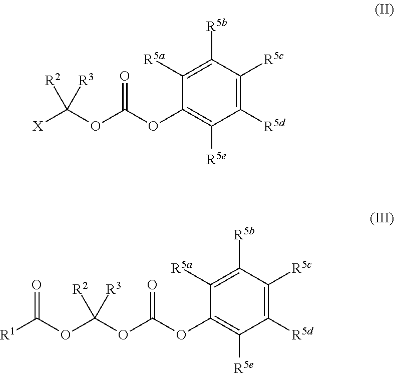

and

(B) reacting the compound of formula (III), or a stereoisomer or a salt thereof, with HNR^{4a}R^{4b} to form the compound of formula (I):

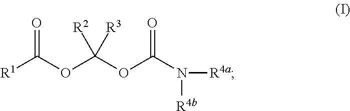

wherein:

each of R¹ and R² is independently C₁₋₄ alkyl;

R³ is H or C₁₋₄ alkyl;

HNR^{4a}R^{4b} is a drug molecule having an amino moiety;

R^{4a} and R^{4a} are groups of the drug molecule attached to the amino moiety;

each of R^{5a}, R^{5b}, R^{5c}, R^{5d}, and R^{5e} is independently selected from a group consisting of H, halo, C₁₋₄ alkyl, halo C₁₋₄ alkyl, phenyl, —C(O)O—C₁₋₄ alkyl, —C(O)—C₁₋₄ alkyl, —S(O)—C₁₋₄ alkyl, CN, —C(O)—NR^{6a}R^{6b}, substituted or unsubstituted C₁₋₄ alkoxy, and substituted or unsubstituted phenoxy;

each of R^{6a} and R^{6b} is independently H, or C₁₋₄ alkyl; or R^{6a} and R^{6b} together with N they are attached to form heterocycle;

provided that at least one of R^{5a}, R^{5b}, R^{5c}, R^{5d}, and R^{5e} is other than H; or any two adjacent R^{5a}, R^{5b}, R^{5c}, R^{5d}, and R^{5e} are joined together to form a carbocycle or heterocycle; and

X is a leaving group.

In some embodiments, the drug molecule HNR^{4a}R^{4b} is a drug molecule containing a primary or a secondary amino group.

In some embodiments, the drug molecule HNR^{4a}R^{4b} is selected from acebutalol, albuterol, alprenolol, atenolol, bunolol, bupropion, butopamine, butoxamine, carbuterol, cartelolol, colterol, deterenol, dexpropanolol, diacetolol, dobutamine, exaprolol, exprenolol, fenoterol, fenyripol, labotolol, levobunolol, metolol, metaproterenol, metoprolol, nadolol, pamatolol, penbutalol, pindolol, pirbuterol, practolol, prenalterol, primidolol, prizidilol, procaterol, propanolol, quinterenol, rimiterol, ritodrine, solotol, soterenol, sulfiniolol, sulfinterol, sulictidil, tazaolol, terbutaline, timolol, tiprenolol, tipridil, tolamolol, thiabendazole, albendazole, albutoin, alendronate, alinidine, alizapride, amiloride, a minorex, aprinocid, cambendazole, cimetidine, cisapride, clonidine, cyclobenzadole, delavirdine, efegatrin, etintidine, fenbendazole, fenmetazole, flubendazole, fludorex, gabapentin, icadronate, lobendazole, mebendazole, metazoline, metoclopramide, methylphenidate, mexiletine, neridronate, nocodazole, oxfendazole, oxibendazole, oxmetidine, pamidronate, parbendazole, pramipexole, prazosin, pregabalin, procainamide, ranitidine, tetrahydrazoline, tiamenidine, tinazoline, tiotidine, tocamide, tolazoline, tramazoline, xylometazoline, dimethoxyphenethylamine, n-[3(R)-[2-piperidin-4-yl)ethyl]-2-piperidone-1-yl]acetyl-3(R)-methyl-β-alanine, adrenolone, aletamine, amidephrine, amphetamine, aspartame, bamethan, betahistine, carbidopa, clorprenaline, chlortermine, dopamine, L-dopa, ephrinephrine, etryptamine, fenfluramine, methyldopamine, norepinephrine, enviroxime, nifedipine, nimodipine, triamterene, pipedemic acid and similar compounds, 1-ethyl-6-fluoro-1,4-dihydro-4-oxo-7-(1-piperazinyl)-1,8-napthyridine-3-carboxylic acid and 1-cyclopropyl-6-fluoro-1,4-dihydro-4-oxo-7-(piperazinyl)-3-quinolinecarboxylic acid, theprubicin, deoxyspergualin, seglitide, nebracetam, benanomicin B, eremomycin, thrazarine, tosufloxacin, baogongteng A, angiopeptin, boholmycin, ravidomycin, tageflar, orienticins, amphotericin B, tiamdipine, doxorubicin, lysobactin, mofegiline, octreotide, oxolide, amikacin, phospholine, nuvanil, cispentacin, chlorotetain, remacemide, ramoplanins, janthinomycins, mersacidin, droxidopa, helvecardin A, helvecardin B, rilmazafone, vigabatrin, amlodipine, (R)-(+)-amlodipine, mideplanin, milnacipran, pranedipine, olradipine, deoxymethylspergualin, fudosteine, trovafloxacin, ceranapril, restricticin, idarubicin, arbekacin, giracodazole, poststatin, pazufloxacin, D-cycloserine, ovothiol A, ceftizoxime, icatibant, p-iodorubidazone, aladapcin, dalargin, seproxetine, pradimicin E, pradimicin FA-2, tafenoquine, sampatrilat, ruboxyl, dactimicin, alatrofloxacin, galarubicin, metaraminol, exatecan, squalamine, paromomycin, leustroducsin A, leustroducsin B, leustroducsin C, lanicemine, azoxybacilin, tetrafibricin, pixantrone, ziconotide, garomefrine, spinorphin, doripenem, alestramustine, seraspenide, safingol, aminolevulinic acid, pelagiomicin C, styloguanidine, L-4-oxalysine, eglumegad, rhodopeptins, mycestericin E, midaxifylline, anisperimus, lagatide, ibutamoren, oritavancin, ecenofloxacin, metyrosine, methyldopa, baclofen, tranylcypromine, micronomicin, zorubicin, epirubicin, gilatide, epithalon, cystamine, pluraflavin A, pluraflavin B, pasireotide, caprazamycin, barusiban, spisulosine, 21-aminoepothilone B, capsavanil, olcegepant, sulphostin, lobophorin A, papuamide A, papuamide B, cystocin, deoxynegamycin, galnon, pyloricidin B, brasilicardin A, neramexane, kaitocephalin, icofungipen, aliskiren, capromorelin, histaprodifen, donitriptan, cambrescidins, tipifarnib, tabimorelin, belactosin A, belactosin C, circinamide, targinine, sulphazocine, nepicastat, oseltamivir, hydrostatin A, butabindide, netamiftide, memantine, fluvoxamine, deferoxamine, tranexamic acid, fortimicin A, cefaclor, lisinopril, ubestatin, cefminox, aspoxicillin, cefcanel, cefcanel daloxate, olamufloxacin, R-(+)-aminoindane, gemifloxacin, kahalalide F, palau'amine, examorelin, leustroducsin H, sabarubicin, amifostine, L-homothiocitrulline, L-thiocitrulline, impentamine, neboglamine, amselamine, cetefloxacin, cyclothialidine, fluvirucin B2, loracarbef, cefprozil, sperabillins, milacamide, avizafone, α-methyltryptophan, cytaramycin, lanomycin, decaplanin, eflornithine, L-histidinol, tuftsin, kanamycin, amthamine, sitafloxacin, leurubicin, amantadine, isodoxorubicin, gludopa, bactobolin, esafloxacin, tabilautide, lazabemide, enalkiren, amrubicin, daunorubicin, mureidomycins, pyridazomycin, cimaterol, (+)-isamoltan, N-desmethylmilameline, noberastine, fosopamine, adaprolol, pradimicin B, amosulalol, xamoterol, boholmycin, risotilide, indeloxazine, denopamine, parodilol, utibapril, nardeterol, biemnidin, sparfloxacin, sibanomicin, tianeptine, oberadilol, methoctramine, sezolamide, anabasine, zilpaterol, zabiciprilat, enkastins, ulifloxacin, (+)-sotalol, deoxynojirimycin, altromycin A, altromycin C, dorzolamide, fepradinol, delapril, ciprofloxacin, balofloxacin, mepindolol, berlafenone, ramipril, dopexamine, dilevalol, (−)-nebivolol, duramycin, enalapril, meluadrine, zelandopam, voglibose, sertraline, carvedilol, pafenolol, paroxetine, fluoxetine, phendioxan, salmeterol, solpecainol, repinotan, bambuterol, safinamide, tilisolol, 7-oxostaurosporine, caldaret, sertraline, cilazapril, benazepril, prisotinol, gatifloxacin, ovothiol B, adaprolol, tienoxolol, fluparoxan, alprenoxime, efegatran, pradimicin, salbostatin, ersentilide, (S)-noremopamil, esperamicin A1, batoprazine, ersentilide, osutidine, quinapril, dihydrexidine, argiopine, pradimicin D, frovatriptan, hispidospermidin, silodosin, michellamine B, sibenadet, tetrindol, talibegron, topixantrone, nortopixantrone, tecalcet, buteranol, α-methylepinephrine, nornicotine, thiofedrine, lenapenem, imidapril, epibatidine, premafloxacin, socorromycin, trandolapril, tamsulosin, dirithromycin, inogatran, vicenistatin, immepyr, immepip, balanol, orbifloxacin, maropitant, dabelotine, lerisetron, ertapenem, nolomirole, moxifloxacin, vofopitant, halofuginone, melagatran, ximelagatran, fasudil, isofagomine, pseudoephedrine, propafenone, celiprolol, carteolol, penbutolol, labetalol, acebutolol, reproterol, rimoterol, amoxapine, maprotiline, viloxazine, protriptyline, nortriptyline, desipramine, oxprenolol, propranolol, ketamine, butofilolol, flecamide, tulobuterol, befunolol, immucillin-H, vestipitant, cinacalcet, lapatinib, desloratadine, ladostigil, vildagliptin, tulathromycin B, becampanel, salbutamol, delucemine, solabegron, paroxetine, gaboxadol, telavancin, ralfinamide, tomoxetine, dalbavancin, elarofiban, ferulinolol, fenoldopam, sumanirole, sarizotan, brinzolamide, pradofloxacin, garenoxacin, reboxetine, ezlopitant, palindore, nebivolol, dinapsoline, proxodolol, repinotan, demexiptiline, mitoxantrone, norfloxacin, dilevalol, nipradilol, esmolol, ibopamine, troxipide, arotinolol, formoterol, bopindolol, cloranolol, mefloquine, perindopril, mabuterol, bisoprolol, bevantolol, betaxolol, tertatolol, enoxacin, lotrafiban, moexipril, droxinavir, adrogolide, alniditan, tigecycline, lubazodone, meropenem, temocapril, napsamycins, (−)-cicloprolol, ecteinascidins, alprafenone, landiolol, tirofiban, noberastine, rasagiline, setazindol, picumeterol, arbutamine, mecamylamine, delfaprazine, imidapril, midafotel, manzamines, binospirone, duloxetine, and litoxetine.

In some embodiments, the drug molecule is any secondary or primary amine drug HNR^{4a}R^{4b} described in various compendia accessible to the skilled artisan, such as, for example, the Merck Index, 13^{th} Edition, 2001 or the Physicians Desk Reference, 59^{th} Edition, 2005. Accordingly, secondary or primary amine drugs HNR^{4a}R^{4b} described in references such as those, supra, are within the ambit of the present description.

In some embodiments, the drug molecule is selected from alendronate, amifostine, rac-baclofen, R-baclofen, carbidopa, clonidine, ciprofloxacin, cisapride, daunorubicin, doxorubicin, fenoldopam, fenoterol, gabapentin, gentamycin, kanamycin, levodopa, meropenem, metazoline, neomycin, pamidronate, pregabalin, tobramycin, trovafloxacin and vigabatrin. In yet other embodiments, the drug molecule HNR^{4a}R^{4b} is gabapentin. In still other embodiments, HNR^{4a}R^{4b} is R-baclofen. In still other embodiments, HNR^{4a}R^{4b} is a GABA analog.

In some embodiments, X is halo.

In some embodiments, X is Cl.

In some embodiments, the reaction step (A) occurs in a solvent.

In another embodiment, the reaction step (A) occurs in absence of any solvent.

In yet another embodiment, the reaction step (A) occurs in an aprotic solvent.

In yet another embodiment, the reaction step (A) occurs in a protic solvent.

In yet another embodiment, the reaction step (A) occurs in a solvent selected from heptane, xylene, toluene, N-methylpyrrolidine, N,N-diisopropylamine, dimethyl formamide, dimethyl sulfoxide, diphenyl ether, and combinations thereof. In some embodiments, the reaction step (A) occurs in heptane, xylene, toluene, or N-methylpyrrolidine. In some embodiments, the reaction step (A) occurs in xylene or heptane. In some embodiments, the reaction step (A) occurs in heptane. In certain embodiments, hepatane is a mixture of heptanes.

In yet another embodiment, the reaction step (A) occurs in a solvent which is inert to the carboxylic acid salt. In one embodiment, the solvent is alcohol (such as methanol, ethanol, isopropanol, or tert-butanol), water, dichloromethane, dichloroethane, dimethylformamide, dimethylacetamide, hexamethylphosphoramide, N-methylpyrrolidinone, dimethyl sulfoxide, pyridine, ethyl acetate, acetone, 2-butanone, methyl-tert-butyl ether, chloroform, acetonitrile, benzene, toluene, xylene or a carboxylic acid (such as the corresponding carboxylic acid), or mixtures thereof. In some embodiments, the reaction takes place at a suitable temperature such as from room temperature to the boiling point of the particular solvent or solvent combination employed.

In some embodiments, the reaction step (A) occurs at a temperature from about 50° C. to about 120° C. In some embodiments, the reaction step (A) occurs at a temperature from about 90° C. to about 120° C. In some embodiments, the reaction step (A) occurs at a temperature from about 115° C. to about 120° C.

In some embodiments, the reaction step (A) occurs by reaction of a metal salt of R¹CO₂H with the compound of formula (II). The cation of the salt can be silver, copper, mercury, sodium, potassium, lithium, caesium, calcium, magnesium or zinc. In certain embodiments, the molar ratio of the carboxylic acid salt to the compound for formula (II) or salt thereof is between 1:1 and 1:20, more particularly between 1:1 and 1:5 and most particularly about 1:1.

In some embodiments, the reaction step (A) occurs in the presence of a metal oxide.

In some embodiments, the reaction step (A) occurs in the presence of a metal oxide; and the metal is silver, copper, mercury, sodium, potassium, lithium, caesium, calcium, magnesium or zinc. In certain embodiments, the reaction step (A) occurs in the presence of Cu₂O. In some embodiments, with respect to the reaction in the presence of Cu₂O, the corresponding carboxylic acid, a mixture of heptane or o-xylene and the corresponding carboxylic acid, a mixture of xylene isomers and ethylbenzene (all contained in the solvent mixture designated “xylenes”) or toluene may be used as a solvent.

In some embodiments, the reaction step (A) occurs in the presence of a metal alkanoate; or a metal salt of R¹CO₂H; and R¹ is as defined herein. In certain embodiments, the metal is silver, copper, mercury, sodium, potassium, lithium, cesium, calcium, magnesium or zinc. In some embodiments, the reaction step (A) occurs in the presence of a silver salt of R¹CO₂H.

In some embodiments, the reaction step (A) occurs in the presence of silver isobutyrate. In certain embodiments, when the reaction step (A) occurs in the presence of silver isobutyrate, the reaction takes place at a suitable temperature, such as 90° C. In certain embodiments, for this step, the corresponding carboxylic acid may be used as a solvent. For example, when R¹ is isopropyl, isobutyric acid may be used as a solvent.

In some embodiments, the reaction step (A) occurs in the presence of R¹C(O)—O—C(O)R¹; and R¹ is as defined herein. In certain embodiments, the reaction step (A) occurs in the presence of isobutyric anhydride.

In some embodiments, the reaction step (A) occurs in the presence of an organic base, such as N,N-diisopropylethylamine, triethylamine, tributylamine, dimethylisopropylamine, N-methylmorpholine, N-methypyrrolidine, N-methylpiperidine, pyridine, 2-methylpyridine, 2,6-methylpyridine, 4-dimethylaminopyridine, 1,4-diazabicyclo[2.2.2]octane, 1,8-diazabicyclo[5.4.0]undec-7-ene or 1,1-diazabicyclo[4.3.0]undec-7-ene or by reaction with a quaternary ammonium salt of the corresponding carboxylic acid, wherein the cation is tetramethylammonium, tetraethylammonium or tetrabutylammonium. In certain embodiments, the molar ratio of the carboxylic acid or the quaternary ammonium salt of the carboxylic acid to the compound of formula (II) or salt thereof is between 1:1 and 1:20, more particularly between 1:1 and 1:5 and most particularly about 1:1. The reaction is typically conducted in a solvent which is inert to the carboxylic acid or quaternary ammonium salt of the carboxylic acid such as alcohol (such as methanol, ethanol, isopropanol, or tert-butanol), water, dichloromethane, dichloroethane, dimethylformamide, dimethylacetamide, hexamethylphosphoramide, N-methylpyrrolidinone, dimethyl sulfoxide, pyridine, ethyl acetate, acetone, 2-butanone, methyl-tert-butyl ether, chloroform, acetonitrile, benzene, toluene, xylene or a carboxylic acid (such as the corresponding carboxylic acid), or mixtures thereof. The reaction takes place at a suitable temperature such as from room temperature to the boiling point of the particular solvent or solvent combination employed. In some embodiments, the organic base is N,N-diisopropylethylamine. In certain embodiments of this step, a catalytic amount of an iodide or bromide salt (e.g. sodium iodide, potassium iodide, tetramethylammonium iodide or tetrabutylammonium iodide or n-tetrabutylammonium bromide, particularly sodium iodide) may be used. The reaction takes place at a suitable temperature, such as 80° C. A mixture of dimethyl carbonate and the isobutyric acid may be used as a solvent.

In some embodiments, the reaction step (A) occurs in the presence of a tetraalkylammonium salt. In certain embodiments, the reaction step (A) occurs in the presence of tetraalkylammonium chloride, tetraalkylammonium bromide, or tetraalkylammonium iodide. In some embodiments, the reaction step (A) occurs in the presence of tetrabutylammonium bromide.

In some embodiments, the reaction step (A) occurs over a period of 0.5 to 10 hr. In some embodiments, the reaction occurs over a period of 1-7 hrs. In some embodiments, the reaction occurs over a period of 3-5 hrs.

In some embodiments, when R³ is H, the reaction step (A) may further comprise preparation of individual stereoisomers or pure enantiomers of the compound of formula (III).

In some embodiments, when R³ is H, the reaction step (A) may further comprise enzymatic resolution of the compound of formula (III). In certain embodiments, the enzymatic resolution occurs in the presence of a suitable enzyme. In some embodiments, the enzyme is an esterase enzyme. In some embodiments, the enzyme is lipase. In some embodiments, the resolution occurs in the presence of a phosphate buffer.

In some embodiments, the enzymatic resolution is carried out by following procedures as described in U.S. Pat. No. 7,872,046 or 8,062,870.

In some embodiments, the enzymatic resolution is used to prepare the compounds according to formulae (III-A) and (III-B):

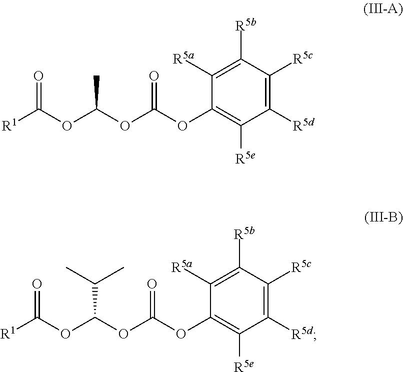

wherein R¹ and R^{5a}-R^{5e} are as defined herein.

In some embodiments, the reaction step (B) occurs in a protic or aprotic solvent; or combinations thereof.

In some embodiments, the reaction step (B) occurs in an alcohol solvent. In some embodiments, the reaction step (B) occurs in a combination of alcohol solvents. In certain embodiments, the alcohol solvent is methanol, ethanol, i-propanol, or n-propanol. In some embodiments, the alcohol solvent is i-propanol or isopropanol.

In another embodiment, the reaction step (B) occurs in heptane, xylene, toluene, dialkyl ether, cyclic ethers, dimethyl formamide, dimethyl sulfoxide, water, acetonitrile, ethyl acetate, or combinations thereof.

In some embodiments, the reaction step (B) occurs in t-butyl methyl ether, heptane, THF, water, acetonitrile, or combinations thereof.

In some embodiments, the reaction step (B) occurs in a mixture of heptane, water, and acetonitrile.

In some embodiments, the reaction step (B) occurs in a mixture of t-butyl methyl ether, water, and acetonitrile.

In certain embodiments, the reaction step (B) occurs at a temperature from about 0° C. to about 80° C., about 10° C. to about 60° C. or about 10° C. to about 50° C. In some embodiments, the reaction step (B) occurs at a temperature from about 10° C. to about 20° C.

In some embodiments, the reaction step (B) occurs in the presence of a base. In some embodiments, the base is an inorganic base. In other embodiments, the base is an organic base.

In some embodiments, the reaction step (B) occurs in the presence of triethylamine, tetramethylguanidine (TMG), aqueous NaOH, aqueous KOH, aqueous Na₂CO₃, aqueous K₂CO₃, aqueous NaHCO₃, aqueous KHCO₃, or mixtures thereof.

In some embodiments, R¹ is methyl, ethyl, isopropyl, or n-propyl.

In some embodiments, R¹ is isopropyl.

In some embodiments, R² is methyl, ethyl, isopropyl or n-propyl.

In some embodiments, R² is methyl or isopropyl.

In some embodiments, R³ is hydrogen.

In some embodiments, one of R^{5a}, R^{5b}, R^{5c}, R^{5d}, and R^{5e} is selected from halo, C₁₋₄ alkyl, —C(O)O—C₁₋₄ alkyl, —C(O)—C₁₋₄ alkyl, —S(O)—C₁₋₄ alkyl, CN, —C(O)—NR^{6a}R^{6b}, or substituted or unsubstituted alkoxy, and the rest are H. In other embodiments, two of R^{5a}, R^{5b}, R^{5c}, R^{5d}, and R^{5e} are independently selected from halo, C₁₋₄ alkyl, —C(O)O—C₁₋₄ alkyl, —C(O)—C₁₋₄ alkyl, —S(O)—C₁₋₄ alkyl, CN, —C(O)—NR^{6a}R^{6b}, or substituted or unsubstituted alkoxy, and the rest are H. In other embodiments, three of R^{5a}, R^{5b}, R^{5c}, R^{5d}, and R^{5e} are independently selected from halo, C₁₋₄ alkyl, —C(O)O—C₁₋₄ alkyl, or substituted or unsubstituted alkoxy, and the rest are H.

In some embodiments, one of R^{5a}, R^{5b}, R^{5c}, R^{5d}, and R^{5e} is —C(O)—NR^{6a}R^{6b}; and each of R^{6a} and R^{6b} is independently H or C₁₋₄ alkyl. In other embodiments, one of R^{5a}, R^{5b}, R^{5c}, R^{5d}, and R^{5e} is —C(O)—NR^{6a}R^{6b}; and R^{6a} and R^{6b} together with N they are attached to form a heterocycle. In some embodiments, one of R^{5a}, R^{5b}, R^{5c}, R^{5d}, and R^{5e} is —C(O)—NH₂, or —C(O)NMe₂. In other embodiments, R^{5c} is —C(O)—NH₂, or —C(O)NMe₂.

In some embodiments, one of R^{5a}, R^{5b}, R^{5c}, R^{5d}, and R^{5e} is —C(O)—C₁₋₄ alkyl. In other embodiments, one of R^{5a}, R^{5b}, R^{5c}, R^{5d}, and R^{5e} is —C(O)Me, or —C(O)Et. In some embodiments, R^{5c} is —C(O)Me, or —C(O)Et.

In some embodiments, one of R^{5a}, R^{5b}, R^{5c}, R^{5d}, and R^{5e} is —S(O)—C₁₋₄ alkyl. In other embodiments, one of R^{5a}, R^{5b}, R^{5c}, R^{5d}, and R^{5e} is —S(O)Me, or —S(O)Et. In some embodiments, R^{5c} is —S(O)Me, or —S(O)Et.

In some embodiments, one of R^{5a}, R^{5b}, R^{5c}, R^{5d}, and R^{5e} is CN. In some embodiments, R^{5c} is CN.

In some embodiments, R^{5a} is halo. In other embodiments, R^{5a} is F, or Cl.

In some embodiments, R^{5a} is F; and each of R^{5b}, R^{5c}, R^{5d}, and R^{5e} is H.

In other embodiments, one, two, three, or four of R^{5a}R^{5b}, R^{5c}, R^{5d}, and R^{5e} is/are independently Cl, F, Me, Et, —C(O)OMe or —C(O)OEt; and the rest are H.

In some embodiments, R^{5c} is —C(O)OMe or Me; and each of R^{5a}, R^{5b}, R^{5d}, and R^{5e} is H.

In some embodiments, R^{5c} is —C(O)Me; and each of R^{5a}, R^{5b}, R^{5d}, and R^{5e} is H.

In some embodiments, R^{5c} is —S(O)OMe or Me; and each of R^{5a}, R^{5b}, R^{5d}, and R^{5e} is H.

In some embodiments, R^{5c} is —S(O)Me; and each of R^{5a}, R^{5b}, R^{5d}, and R^{5e} is H.

In some embodiments, R^{5c} is —C(O)—NH₂, or —C(O)NMe₂; and each of R^{5a}, R^{5b}, R^{5d}, and R^{5e} is H.

In some embodiments, R^{5c} is CN; and each of R^{5a}, R^{5b}, R^{5d}, and R^{5e} is H.

In other embodiments, R^{5b} is —OMe; and each of R^{5a}, R^{5e}, R^{5d}, and R^{5e} is H.

In other embodiments, any two of R^{5a}, R^{5b}, R^{5c}, R^{5d}, and R^{5e} form —O—CH₂—O—, or —O—CH₂—CH₂—O—; and the rest are H.

In other embodiments, each of R^{5a}, R^{5b}, R^{5c}, R^{5d}, or R^{5e} is independently selected from H, halo, C₁₋₄ alkyl, halo C₁₋₄ alkyl, phenyl, —C(O)O—C₁₋₄ alkyl, —C(O)—C₁₋₄ alkyl, —S(O)—C₁₋₄ alkyl, CN, —C(O)—NR^{6a}R^{6b}, substituted or unsubstituted C₁₋₄ alkoxy, and substituted or unsubstituted phenoxy; and provided that the pKa of the starting phenol (II′) is about 7-11.

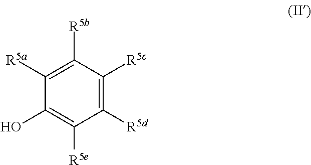

In other embodiments, the pKa is about 8 to 10. In some embodiments, the pKa is about 8 to 9. In other embodiments, the pKa is about 8.1 to 8.5. In some embodiments, the pKa is about 8, 8.1, 8.2, 8.3, 8.4, or 8.5.

In some embodiments, when R^{5a} is fluoro and R^{5b}, R^{5c}, R^{5d} and R^{5e} are hydrogen, the step (B) takes place in the presence of a base such as aqueous sodium hydroxide in a solvent such as tetrahydrofuran. In other embodiments, triethylamine base could be used and a mixture of water, acetonitrile and heptanes could be used as the solvent. In some embodiments, the reaction takes place at a temperature from 10° C. to 30° C.

In some embodiments, when R^{5a} is chloro and R^{5b}, R^{5c}, R^{5d} and R^{5e} are hydrogen, the step (B) takes place in the presence of a base such as aqueous sodium hydroxide in a solvent such as tetrahydrofuran, and at a temperature from 0° C. to 50° C.

In other embodiments, R^{5b} is halo and R^{5a}, R^{5c}, R^{5d} and R^{5e} are hydrogen. In some embodiments, R^{5b} is fluoro and R^{5a}, R^{5c}, R^{5d} and R^{5e} are hydrogen.

In some embodiments, when R^{5b} is fluoro and R^{5a}, R^{5c}, R^{5d} and R^{5e} are hydrogen, the step (B) takes place in the presence of a base such as triethylamine in a solvent such as a mixture of water, acetonitrile and tert-butyl methyl ether, and at a temperature from 10° C. to 20° C.

In some embodiments, R^{5c} is halo and R^{5a}, R^{5b}, R^{5d} and R^{5e} are hydrogen. In some embodiments, R^{5c} is fluoro and R^{5a}, R^{5b}, R^{5d} and R^{5e} are hydrogen.

In some embodiments, when R^{5c} is fluoro and R^{5a}, R^{5b}, R^{5d} and R^{5e} are hydrogen, the step (B) takes place in the presence of a base such as triethylamine in a solvent such as a mixture of water, acetonitrile and tert-butyl methyl ether, and at a temperature from 0° C. to 20° C.

In some embodiments, two of R^{5a}, R^{5b}, R^{5c}, R^{5d} and R^{5e} are halo and the remaining groups are hydrogen. In some embodiments, two of R^{5a}, R^{5b}, R^{5c}, R^{5d} and R^{5e} are fluoro and the remaining groups are hydrogen. In some embodiments, R^{5a} and R^{5e} are fluoro and R^{5b}, R^{5c} and R^{5d} are hydrogen.

In some embodiments, when R^{5a} and R^{5e} are fluoro and R^{5b}, R^{5c} and R^{5d} are hydrogen, the step (B) takes place in a solvent such as a mixture of water and acetonitrile, at a temperature from 10° C. to 60° C.

In some embodiments, one or more of R^{5a}, R^{5b}, R^{5c}, R^{5d} and R^{5e} is —OC₁₋₃ alkyl and the remaining groups are hydrogen. In some embodiments, one of R^{5a}, R^{5b}, R^{5c}, R^{5d} and R^{5e} is methoxy and the remaining groups are hydrogen.

In some embodiments, R^{5b} is —OC₁₋₃alkyl and R^{5a}, R^{5c}, R^{5d} and R^{5e} are hydrogen. In some embodiments, R^{5b} is methoxy and R^{5a}, R^{5c}, R^{5d} and R^{5e} are hydrogen.

In some embodiments, when R^{5b} is methoxy and R^{5a}, R^{5c}, R^{5d} and R^{5e} are hydrogen, the step (B) takes place in the presence of a base such as aqueous sodium hydroxide and tetramethylguanidine in a solvent such as a tetrahydrofuran, at a temperature of 20° C.

In some embodiments, R^{4a} is H.

In some embodiments, R^{4b} is

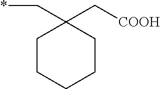 [SMILES: *CC1(CC(=O)O)CCCCC1]

and wherein the * represents the attachment point.

In other embodiments, R^{4b} is

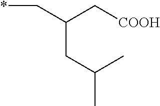 [SMILES: *CC(CC(=O)O)CC(C)C]

and wherein the * represents the attachment point.

In other embodiments, R^{4b} is

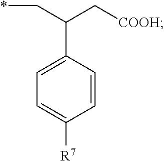

wherein the * represents the attachment point; and R⁷ is F or Cl.

In some embodiments, R⁷ is F. In other embodiments, R⁷ is Cl.

In some embodiments, R^{4b} is

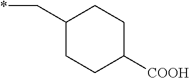 [SMILES: *CC1CCC(C(=O)O)CC1]

wherein the * represents the attachment point.

In some embodiments, with respect to the compound of formula (I), the compound is a compound according to formula (IVa), (IVb), (IVc), or (IVd):

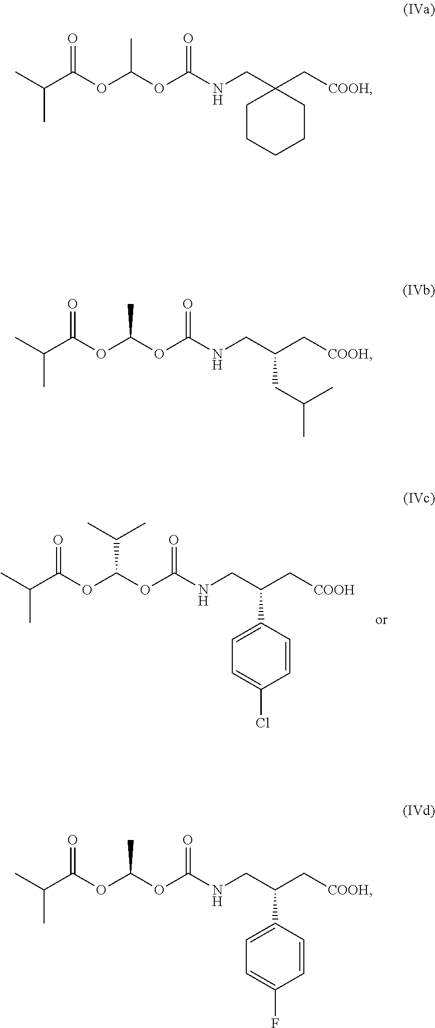 [SMILES: CC(OC(=O)NCC1(CC(=O)O)CCCCC1)OC(=O)C(C)C ; CC(C)C[C@H](CNC(=O)O[C@H](C)OC(=O)C(C)C)CC(=O)O ; CC(C)C(=O)O[C@@H](OC(=O)NC[C@H](CC(=O)O)c1ccc(Cl)cc1)C(C)C ; CC(C)C(=O)O[C@@H](C)OC(=O)NC[C@H](CC(=O)O)c1ccc(F)cc1]

or a salt thereof.

In some embodiments, with respect to the compound of formula (I), the compound is a compound according to formula (IVe):

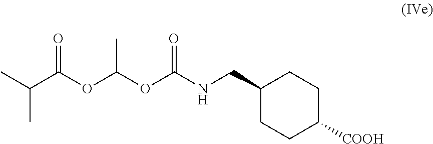 [SMILES: CC(OC(=O)NC[C@H]1CC[C@H](C(=O)O)CC1)OC(=O)C(C)C]

or a salt thereof.

In another aspect, the process of the present disclosure is for the preparation of 1-{[(α-isobutanoyloxyethoxy)carbonyl]aminomethyl}-1-cyclohexane acetic acid or a salt thereof. In some embodiments, the product of the process of the present disclosure is 1-{[(α-isobutanoyloxyethoxy)carbonyl]aminomethyl}-1-cyclohexane acetic acid. In some embodiments, the process of the present disclosure is for the preparation of crystalline 1-{[(α-isobutanoyloxyethoxy)carbonyl]aminomethyl}-1-cyclohexane acetic acid. Crystalline 1-{[(α-isobutanoyloxyethoxy)carbonyl]aminomethyl}-1-cyclohexane acetic acid may be prepared from 1-{[(α-isobutanoyloxyethoxy)carbonyl]aminomethyl}-1-cyclohexane acetic acid as described in PCT Publication No. WO 2005/037784, the contents of which are incorporated herein by reference. In some embodiments, crystallisation is induced by seeding a solution of 1-{[(α-isobutanoyloxyethoxy)carbonyl]aminomethyl}-1-cyclohexane acetic acid with crystals of 1-{[(α-isobutanoyloxyethoxy)carbonyl]aminomethyl}-1-cyclohexane acetic acid.

In another aspect, the present disclosure provides a compound according to formula (III):

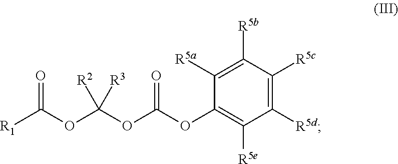

or a stereoisomer thereof, a diastereomer thereof; or a salt of any one of foregoing;
wherein each R¹ and R² is independently C₁₋₄ alkyl; R³ is H or C₁₋₄ alkyl;
and each of R^{5a}, R^{5b}, R^{5c}, R^{5d}, or R^{5e} is independently selected from H, halo, C₁₋₄ alkyl, halo C₁₋₄ alkyl, phenyl, —C(O)O—C₁₋₄ alkyl, —C(O)—C₁₋₄ alkyl, —S(O)—C₁₋₄ alkyl, CN, —C(O)—NR^{6a}R^{6b}, substituted or unsubstituted C₁₋₄ alkoxy, and substituted or unsubstituted phenoxy;
each R^{6a} and R^{6b} is independently H, or C₁₋₄ alkyl; or R^{6a} and R^{6b} together with the N they are attached to form heterocycle;
provided that at least one of R^{5a}, R^{5b}, R^{5c}, R^{5d}, and R^{5e} is other than H; or
any two of R^{5a}, R^{5b}, R^{5c}, R^{5d}, and R^{5e} form —O—CH₂—O—, or —O—CH₂—CH₂—O—.

In some embodiments, R¹ is Me, Et, n-Pr, i-Pr, n-Bu, i-Bu, sec-Bu, or t-Bu.

In some embodiments, R¹ is i-Pr.

In some embodiments, R³ is H, Me, Et, n-Pr, i-Pr, n-Bu, i-Bu, sec-Bu, or t-Bu.

In some embodiments, R³ is H.

In some embodiments, with respect to the compound of formula (III), the compound is a compound according to formula (V):

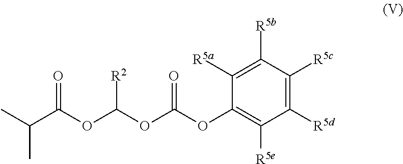

or a stereoisomer thereof, a diastereomer thereof; or a salt of any one of foregoing;
and wherein R², R^{5a}, R^{5b}, R^{5c}, R^{5d}, and R^{5e} are as defined herein.

In some embodiments, with respect to the compound of formula (III) or (V), R² is Me, Et, n-Pr, i-Pr, n-Bu, i-Bu, sec-Bu, or t-Bu. In some embodiments, R² is Me or i-Pr.

In some embodiments, with respect to the compound of formula (III), the compound is a compound according to formula (VIa) or (VIb):

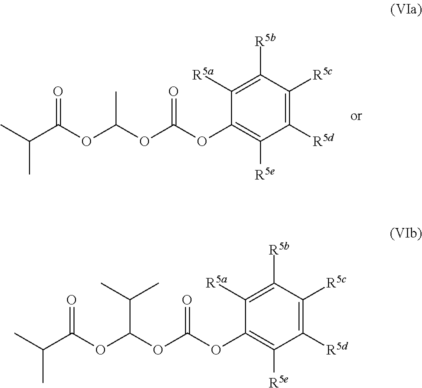

or a stereoisomer thereof, a diastereomer thereof; or a salt of any one of foregoing;
wherein R^{5a}, R^{5b}, R^{5c}, R^{5d}, and R^{5e} are as defined herein.

In some embodiments, with respect to the compound of formula (III), (V), (VIa) or (VIb), one of R^{5a}, R^{5b}, R^{5c}, R^{5d}, and R^{5e} is halo, C₁₋₄ alkyl, —C(O)O—C₁₋₄ alkyl, or substituted or unsubstituted alkoxy, and the rest are H. In other embodiments, two of R^{5a}, R^{5b}, R^{5c}, R^{5d}, and R^{5e} are independently halo, C₁₋₄ alkyl, —C(O)O—C₁₋₄ alkyl, —C(O)—C₁₋₄ alkyl, —S(O)—C₁₋₄ alkyl, CN, —C(O)—NR^{6a}R^{6b}, or substituted or unsubstituted alkoxy, and the rest are H. In other embodiments, three of R^{5a}, R^{5b}, R^{5c}, R^{5d}, and R^{5e} are independently halo, C₁₋₄ alkyl, —C(O)O—C₁₋₄ alkyl, or substituted or unsubstituted alkoxy, and the rest are H.

In some embodiments, with respect to the compound of formula (III), (V), (VIa) or (VIb), R^{5a} is halo. In other embodiments, R^{5a} is F, or Cl.

In some embodiments, with respect to the compound of formula (III), (V), (VIa) or (VIb), R^{5a} is F; and each of R^{5b}, R^{5c}, R^{5d}, and R^{5e} is H.

In other embodiments, with respect to the compound of formula (III), (V), (VIa) or (VIb), one, two, three, or four of R^{5a}, R^{5b}, R^{5c}, R^{5d}, and R^{5e} is/are independently Cl, F, Me, Et, —C(O)OMe, —C(O)OEt, —COMe, —SOMe, —CONH₂, CONMe₂, or —CN; and the rest are H.

In some embodiments, with respect to the compound of formula (III), (V), (VIa) or (VIb), R^{5c} is —C(O)OMe or Me; and each of R^{5a}, R^{5b}, R^{5d}, and R^{5e} is H.

In some embodiments, with respect to the compound of formula (III), (V), (VIa) or (VIb), R^{5c} is —C(O)Me or —S(O)Me; and each of R^{5a}, R^{5b}, R^{5d}, and R^{5e} is H.

In some embodiments, with respect to the compound of formula (III), (V), (VIa) or (VIb), R^{5c} is —C(O)—NH₂, or —C(O)NMe₂; and each of R^{5a}, R^{5b}, R^{5d}, and R^{5e} is H.

In some embodiments, with respect to the compound of formula (III), (V), (VIa) or (VIb), R^{5c} is CN; and each of R^{5a}, R^{5b}, R^{5d}, and R^{5e} is H.

In some embodiments, with respect to the compound of formula (III), (V), (VIa) or (VIb), R^{5b} is —OMe; and each of R^{5a}, R^{5e}, R^{5d}, and R^{5e} is H.

In some embodiments, with respect to the compound of formula (III), (V), (VIa) or (VIb), one of R^{5a}, R^{5b}, R^{5c}, R^{5d}, and R^{5e} is halo, and the rest are H.

In some embodiments, with respect to the compound of formula (III), (V), (VIa) or (VIb), two of R^{5a}, R^{5b}, R^{5c}, R^{5d}, and R^{5e} are halo, and the rest are H.

In some embodiments, with respect to the compound of formula (III), (V), (VIa) or (VIb), three of R^{5a}, R^{5b}, R^{5c}, R^{5d}, and R^{5e} are halo, and the rest are H.

In some embodiments, with respect to the compound of formula (III), (V), (VIa) or (VIb), R^{5a}, R^{5b}, or R^{5e} is Cl or F.

In some embodiments, with respect to the compound of formula (III), (V), (VIa) or (VIb), R^{5a} is Cl or F.

In some embodiments, with respect to the compound of formula (III), (V), (VIa) or (VIb), R^{5b} is Cl or F.

In some embodiments, with respect to the compound of formula (III), (V), (VIa) or (VIb), R^{5c} is Cl or F.

In some embodiments, with respect to the compound of formula (III), (V), (VIa) or (VIb), each of R^{5a} and R^{5e} is Cl or F.

In some embodiments, with respect to the compound of formula (III), (V), (VIa) or (VIb), one of R^{5a}, R^{5b}, R^{5c}, R^{5d}, and R^{5e} is independently Cl, F, Me, Et, C(O)OMe or C(O)OEt, and the rest are H.

In some embodiments, with respect to the compound of formula (III), (V), (VIa) or (VIb), R^{5c} is C(O)OMe or Me.

In some embodiments, with respect to the compound of formula (III), (V), (VIa) or (VIb), one of R^{5a}, R^{5b}, R^{5c}, R^{5d}, and R^{5e} is OMe, and the rest are H.

In some embodiments, with respect to the compound of formula (III), (V), (VIa) or (VIb), R^{5c} is OMe.

In some embodiments, with respect to the compound of formula (III), (V), (VIa) or (VIb), one or two of R^{5a}, R^{5b}, R^{5c}, R^{5d}, and R^{5e} is/are CF₃, and the rest are H.

In some embodiments, with respect to the compound of formula (III), (V), (VIa) or (VIb), R^{5a} is —CF₃. In other embodiments, R^{5e} is CF₃.

In some embodiments, with respect to the compound of formula (III), (V), (VIa) or (VIb), any two of R^{5a}, R^{5b}, R^{5c}, R^{5d}, and R^{5e} form O—CH₂—O—, or O—CH₂—CH₂—O—.

In some embodiments, with respect to the compound of formula (III), (V), (VIa) or (VIb), R^{5a} and R^{5b} form O—CH₂—O—.

In some embodiments, with respect to the compound of formula (III), (V), (VIa) or (VIb), R^{5b} and R^{5e} form O—CH₂—O—.

In some embodiments, with respect to the compound of formula (III), (V), (VIa) or (VIb), R^{5a} and R^{5b} form O—CH₂—CH₂—O—.

In some embodiments, with respect to the compound of formula (III), (V), (VIa) or (VIb), R^{5b} and R^{5e} form O—CH₂—CH₂—O—.

In some embodiments, with respect to the compound of formula (III), the compound is a compound according to formula (VIIa), (VIIb), (VIIc), (VIId), (VIIe), (VIIf), (VIIg), or (VIIh):

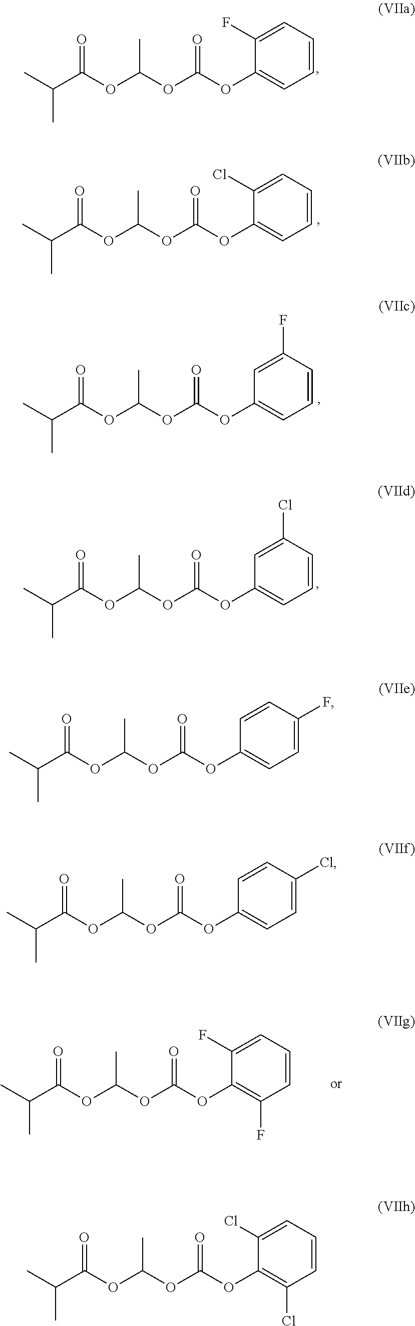

or a stereoisomer thereof, a diastereomer thereof; or a salt of any one of foregoing.

In some embodiments, with respect to the compound of formula (III), the compound is a compound according to formula (VIIIa), (VIIIb), (VIIIc), (VIIId), (VIIIe), or (VIIIf):

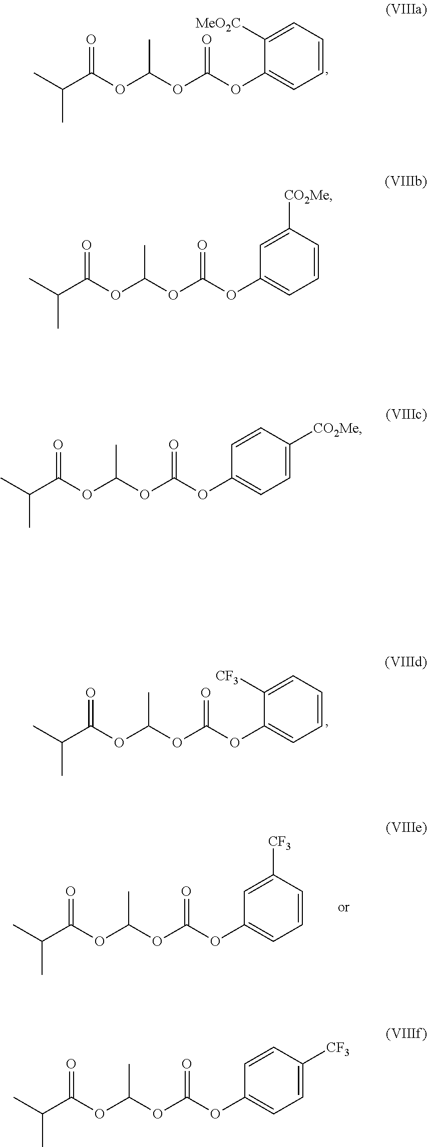 [SMILES: COC(=O)c1ccccc1OC(=O)OC(C)OC(=O)C(C)C ; COC(=O)c1cccc(OC(=O)OC(C)OC(=O)C(C)C)c1 ; COC(=O)c1ccc(OC(=O)OC(C)OC(=O)C(C)C)cc1 ; CC(OC(=O)Oc1ccccc1C(F)(F)F)OC(=O)C(C)C ; CC(OC(=O)Oc1cccc(C(F)(F)F)c1)OC(=O)C(C)C ; CC(OC(=O)Oc1ccc(C(F)(F)F)cc1)OC(=O)C(C)C]

or a stereoisomer thereof, a diastereomer thereof; or a salt of any one of foregoing.

In some embodiments, with respect to the compound of formula (III), the compound is a compound according to formula (IXa), (IXb), (IXc), (IXd), (IXe), (IXf), (IXg), or (IXh):

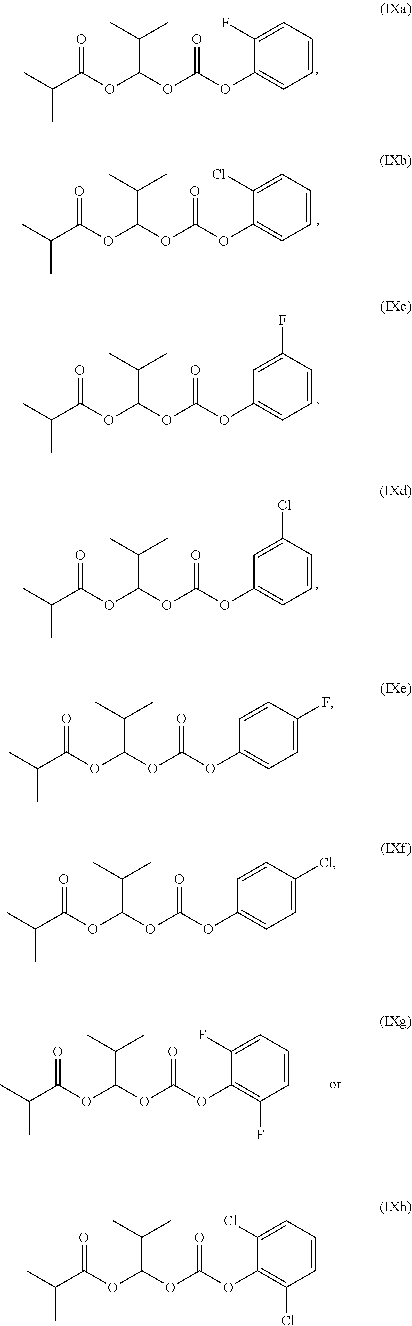

or a stereoisomer thereof, a diastereomer thereof; or a salt of any one of foregoing.

In some embodiments, with respect to the compound of formula (III), the compound is a compound according to formula (Xa), (Xb), (Xc), (Xd), (Xe), or (Xf):

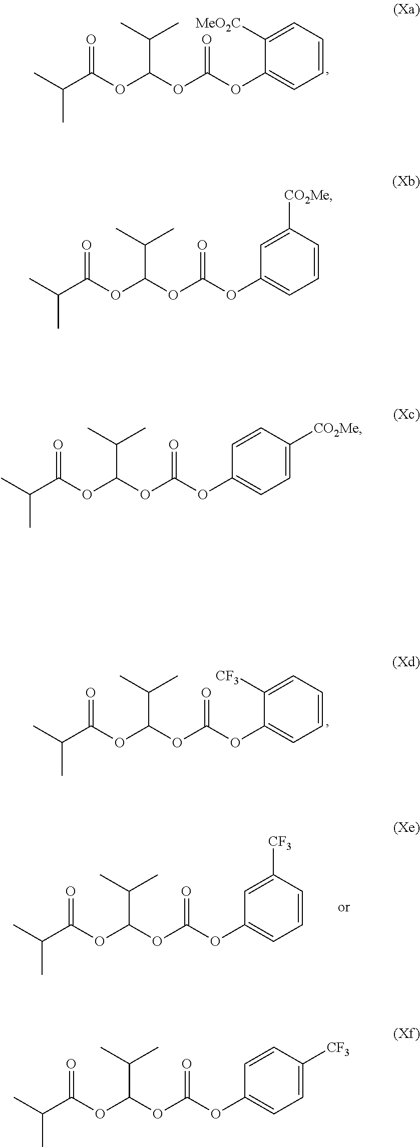 [SMILES: COC(=O)c1ccccc1OC(=O)OC(OC(=O)C(C)C)C(C)C ; COC(=O)c1cccc(OC(=O)OC(OC(=O)C(C)C)C(C)C)c1 ; COC(=O)c1ccc(OC(=O)OC(OC(=O)C(C)C)C(C)C)cc1 ; CC(C)C(=O)OC(OC(=O)Oc1ccccc1C(F)(F)F)C(C)C ; CC(C)C(=O)OC(OC(=O)Oc1cccc(C(F)(F)F)c1)C(C)C ; CC(C)C(=O)OC(OC(=O)Oc1ccc(C(F)(F)F)cc1)C(C)C]

or a stereoisomer thereof, a diastereomer thereof; or a salt of any one of foregoing.

In some embodiments, with respect to the compound of formula (III), the compound is a compound according to formula (XIa), (XIb), (XIc), (XId), (XIe), (XIf), (XIg), or (XIh):

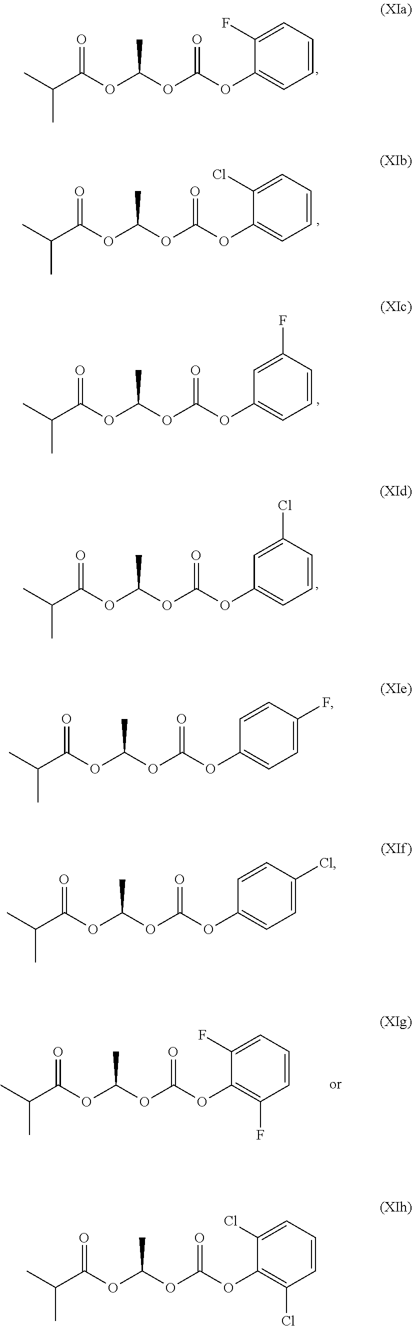

a stereoisomer thereof, a diastereomer thereof; or a salt thereof.

In some embodiments, with respect to the compound of formula (III), the compound is a compound according to formula (XIIa), (XIIb), (XIIc), (XIId), (XIIe), or (XIIf):

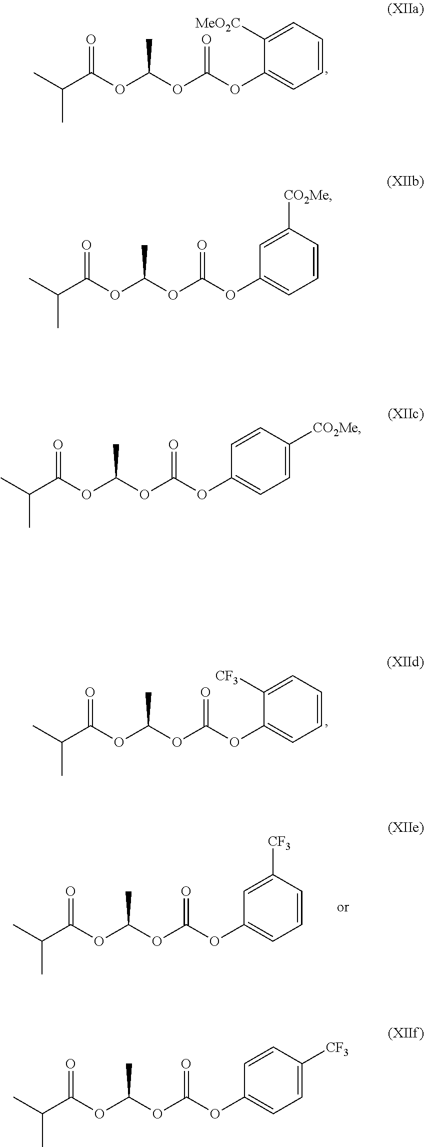 [SMILES: COC(=O)c1ccccc1OC(=O)O[C@H](C)OC(=O)C(C)C ; COC(=O)c1cccc(OC(=O)O[C@H](C)OC(=O)C(C)C)c1 ; COC(=O)c1ccc(OC(=O)O[C@H](C)OC(=O)C(C)C)cc1 ; CC(C)C(=O)O[C@@H](C)OC(=O)Oc1ccccc1C(F)(F)F ; CC(C)C(=O)O[C@@H](C)OC(=O)Oc1cccc(C(F)(F)F)c1 ; CC(C)C(=O)O[C@@H](C)OC(=O)Oc1ccc(C(F)(F)F)cc1]

a stereoisomer thereof, a diastereomer thereof; or a salt thereof.

In some embodiments, with respect to the compound of formula (III), the compound is a compound according to formula (XIIIa), (XIIIb), (XIIIc), (XIIId), (XIIIe), (XIIIf), (XIIIg), or (XIIIh):

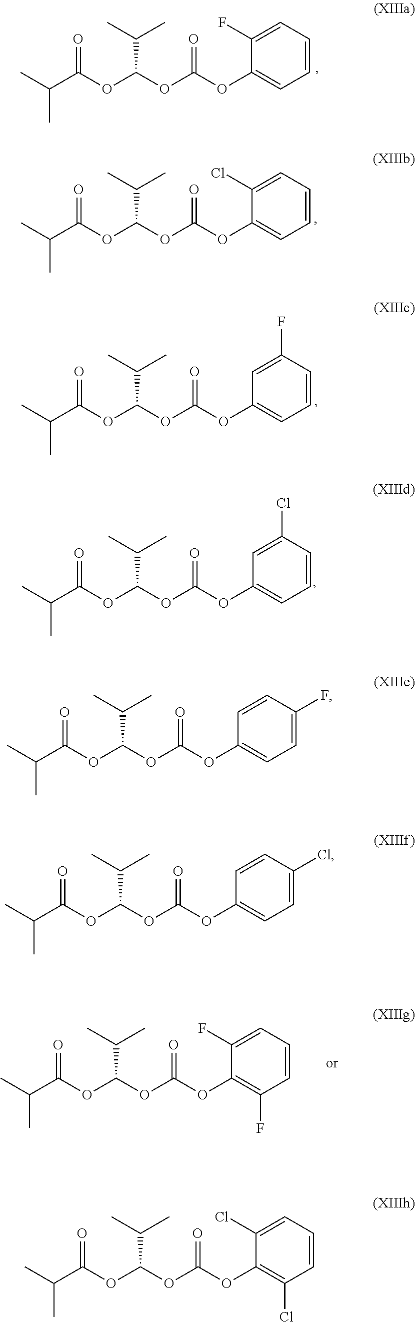

a stereoisomer thereof, a diastereomer thereof; or a salt thereof.

In some embodiments, with respect to the compound of formula (III), the compound is a compound according to formula (XIVa), (XIVb), (XIVc), (XIVd), (XIVe), or (XIVf):

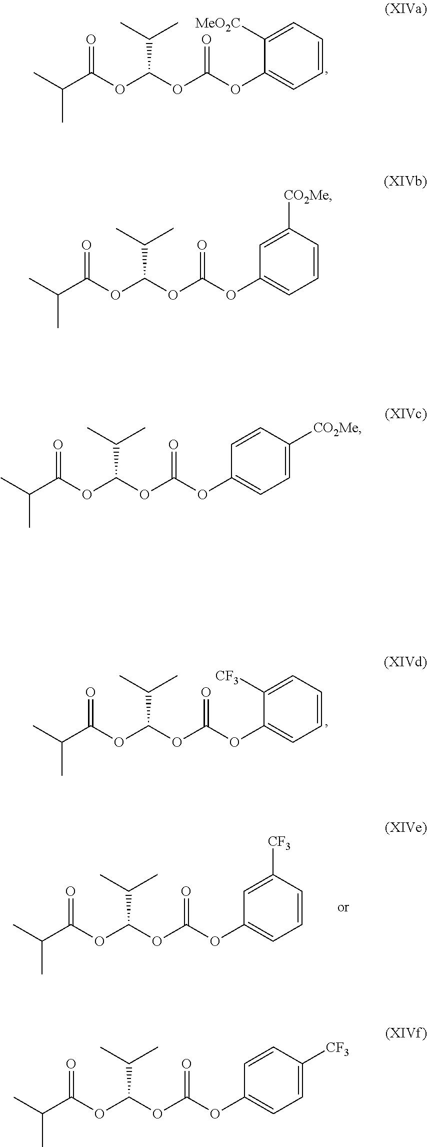 [SMILES: COC(=O)c1ccccc1OC(=O)O[C@H](OC(=O)C(C)C)C(C)C ; COC(=O)c1cccc(OC(=O)O[C@H](OC(=O)C(C)C)C(C)C)c1 ; COC(=O)c1ccc(OC(=O)O[C@H](OC(=O)C(C)C)C(C)C)cc1 ; CC(C)C(=O)O[C@@H](OC(=O)Oc1ccccc1C(F)(F)F)C(C)C ; CC(C)C(=O)O[C@@H](OC(=O)Oc1cccc(C(F)(F)F)c1)C(C)C ; CC(C)C(=O)O[C@@H](OC(=O)Oc1ccc(C(F)(F)F)cc1)C(C)C]

a stereoisomer thereof, a diastereomer thereof; or a salt thereof.

In some embodiments, with respect to the compound of formula (III), the compound is a compound according to formula (XVa), (XVb), (XVc), (XVd), (XVe), or (XVf):

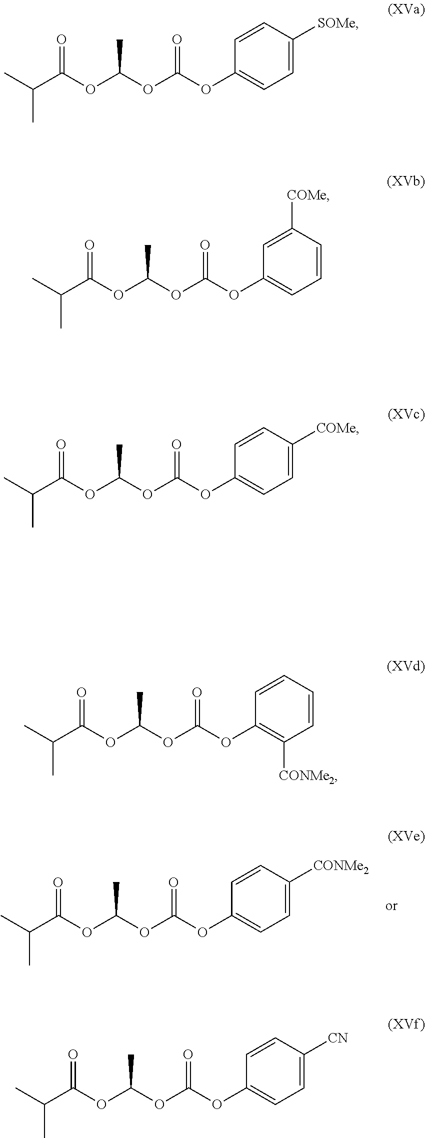

a stereoisomer thereof, a diastereomer thereof; or a salt thereof.

In some embodiments, with respect to the compound of formula (III), the compound is a compound according to formula (XVIa), (XVIb), (XVIc), (XVId), (XVIe), or (XVIf):

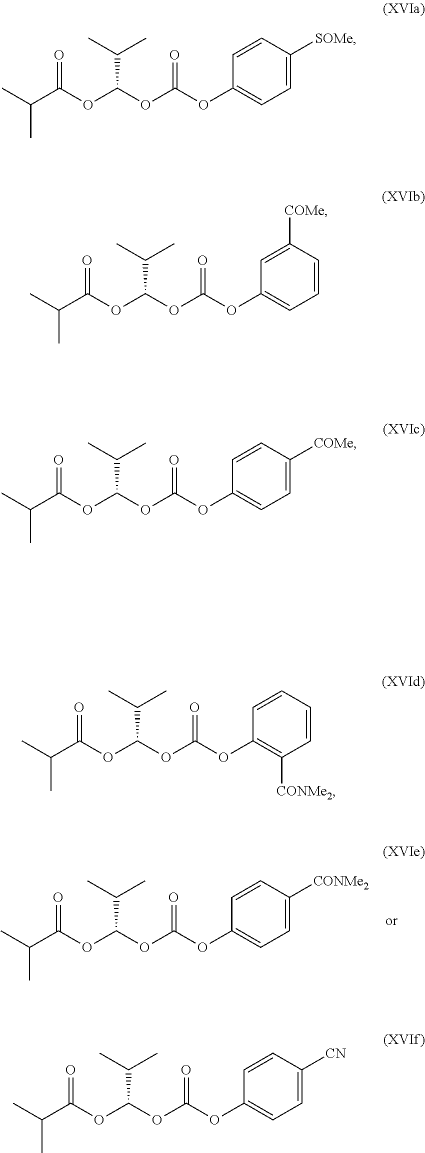

a stereoisomer thereof, a diastereomer thereof; or a salt thereof.

In some embodiments, with respect to the compound of formula (III), the compound may form solvates (e.g. hydrates).

Certain compounds and salts used in the process of the present disclosure may form solvates (e.g. hydrates).

In the context of this disclosure, reference to a salt of a compound encompasses all possible stoichiometric and non-stoichiometric forms of that salt. Because of their potential use in medicine, in some embodiments, the salts of the compound of formula (I) are pharmaceutically acceptable.

Pharmaceutically acceptable base addition salts of compounds of formula (I) include metal salts (such as sodium, potassium, aluminum, calcium, magnesium and zinc) and ammonium salts (such as isopropylamine, diethylamine, and diethanolamine salts). Such salts may be prepared by the skilled chemist, by treating a compound of formula (I) with the appropriate base in a suitable solvent, followed by crystallisation and filtration.

Certain compounds used in the process of the present disclosure are capable of existing in stereoisomeric forms. It will be understood that reference to these compounds encompasses all geometric and optical isomers of these compounds and the mixtures thereof including racemates. The present disclosure also extends to any tautomeric forms and mixtures thereof.

Certain processes of the present disclosure are beneficially conducted as continuous processes. Additionally, the mass efficiency (calculated by dividing the mass of product by the mass of starting materials) of certain processes of the present disclosure is high (higher mass efficiencies are more environmentally friendly). Specifically, where the compound of formula (III) is 2-chlorophenol, mass efficiencies of 2.0% without solvent recovery, or 3.0% with solvent recovery can be achieved. In combination with the cost of starting materials, these features combine to make processes of the present disclosure commercially attractive.

In another aspect, the present disclosure provides a compound of formula (I) or a salt thereof obtainable by the processes of the present disclosure. The present disclosure also provides a pharmaceutical composition which comprises a compound of formula (I) obtained by the processes of the present disclosure, or a pharmaceutically acceptable salt thereof, and a pharmaceutically acceptable carrier. The present disclosure also provides a compound of formula (I), or a pharmaceutically acceptable salt thereof, obtained by the processes of the present disclosure for use as a therapeutic substance in the treatment of epilepsy, pain (particularly neuropathic pain such as post-herpetic neuralgia or diabetic painful neuropathy, or pain associated with irritable bowel syndrome), anxiety (particularly general anxiety disorder), alcohol dependency (ethanol withdrawal syndrome), restless legs syndrome, migraine prophylaxis, fibromyalgia, hot flashes (particularly hot flashes associated with the menopause) and essential tremor.

In some embodiments, the compound of formula (III) is selected from:
1-({[(2-Fluorophenyl)oxy]carbonyl}oxy)ethyl 2-methylpropanoate;
1-({[(3-Fluorophenyl)oxy]carbonyl}oxy)ethyl 2-methylpropanoate;
1-({[(4-Fluorophenyl)oxy]carbonyl}oxy)ethyl 2-methylpropanoate;
1-({[(2,6-Difluorophenyl)oxy]carbonyl}oxy)ethyl 2-methylpropanoate;
1-({[(2-Chlorophenyl)oxy]carbonyl}oxy)ethyl 2-methylpropanoate; and
1-[({[3-(Methyloxy)phenyl]oxy}carbonyl)oxy]ethyl 2-methylpropanoate.

Additional embodiments within the scope provided herein are set forth in non-limiting fashion elsewhere herein and in the examples. It should be understood that these examples are for illustrative purposes only and are not to be construed as limiting in any manner.

### GENERAL SYNTHETIC PROCEDURES

The compounds, intermediates and starting materials provided herein can be purchased or prepared from readily available starting materials using general methods and procedures. See, e.g., Synthetic Schemes below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group, as well as suitable conditions for protection and deprotection, are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography or HPLC. The compounds provided herein may be prepared from known or commercially available starting materials and reagents by one skilled in the art of organic synthesis.

The enantiomerically pure compounds provided herein may be prepared according to any techniques known to those of skill in the art. For instance, they may be prepared by chiral or asymmetric synthesis from a suitable optically pure precursor, or obtained from a racemate by any conventional technique, for example, by chromatographic resolution using a chiral column, TLC or by the preparation of diastereoisomers, separation thereof and regeneration of the desired enantiomer. See, e.g., “Enantiomers, Racemates and Resolutions,” by J. Jacques, A. Collet, and S. H. Wilen, (Wiley-Interscience, New York, 1981); S. H. Wilen, A. Collet, and J. Jacques, Tetrahedron, 2725 (1977); E. L. Eliel Stereochemistry of Carbon Compounds (McGraw-Hill, NY, 1962); and S. H. Wilen Tables of Resolving Agents and Optical Resolutions 268 (E. L. Eliel ed., Univ. of Notre Dame Press, Notre Dame, Ind., 1972, Stereochemistry of Organic Compounds, Ernest L. Eliel, Samuel H. Wilen and Lewis N. Manda (1994 John Wiley & Sons, Inc.), and Stereoselective Synthesis A Practical Approach, Mihály Nográdi (1995 VCH Publishers, Inc., NY, N.Y.).

In certain embodiments, an enantiomerically pure compound of formula (I) may be obtained by reaction of the racemate with a suitable optically active acid or base. Suitable acids or bases include those described in Bighley et al., 1995, Salt Forms of Drugs and Adsorption, in Encyclopedia of Pharmaceutical Technology, vol. 13, Swarbrick & Boylan, eds., Marcel Dekker, New York; ten Hoeve & H. Wynberg, 1985, Journal of Organic Chemistry 50:4508-4514; Dale & Mosher, 1973, J. Am. Chem. Soc. 95:512; and CRC Handbook of Optical Resolution via Diastereomeric Salt Formation, the contents of which are hereby incorporated by reference in their entireties.

Enantiomerically pure compounds can also be recovered either from the crystallized diastereomer or from the mother liquor, depending on the solubility properties of the particular acid resolving agent employed and the particular acid enantiomer used. The identity and optical purity of the particular compound so recovered can be determined by polarimetry or other analytical methods known in the art. The diasteroisomers can then be separated, for example, by chromatography or fractional crystallization, and the desired enantiomer regenerated by treatment with an appropriate base or acid. The other enantiomer may be obtained from the racemate in a similar manner, or worked up from the liquors of the first separation. In certain embodiments, enantiomerically pure compound can be separated from racemic compound by chiral chromatography. Various chiral columns and eluents for use in the separation of the enantiomers are available and suitable conditions for the separation can be empirically determined by methods known to one of skill in the art. Examples of chiral columns available for use in the separation of the enantiomers provided herein include, but are not limited to, CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK.

Compounds disclosed herein may be obtained via the general synthetic methods illustrated in the synthetic schemes presented herein. General synthetic methods useful in the synthesis of compounds, precursors, and starting materials described herein are available in the art. Starting materials useful for preparing compounds and intermediates thereof, and/or practicing methods described herein, are commercially available or may be prepared by well-known synthetic methods.

Additionally, as will be apparent to those skilled in the art, conventional protecting groups or protecting strategies may be necessary to prevent certain functional groups from undergoing undesired reactions. Suitable protecting groups for various functional groups as well as suitable conditions for protecting and protecting particular functional groups are well known in the art.

It will be appreciated that where typical or preferred process conditions, e.g., reaction temperatures, reaction times, molar ratios of reactants, solvents, pressures, etc., are given other process conditions may also be used. Optimal reaction conditions may vary with the particular reactants, solvents, functional groups, and protecting groups used, but such conditions may be determined by one skilled in the art by routine optimization procedures.

Furthermore, certain compounds provided by the present disclosure will contain one or more stereogenic centers. Accordingly, and if desired, such compounds may be prepared or isolated as pure stereoisomers, e.g., as individual enantiomers, diastereomers, atropisomers, rotamers, or as stereoisomer enriched mixtures or racemates. All such stereoisomers are included within the scope of this disclosure. Pure stereoisomers (or enriched mixtures thereof) may be prepared using, for example, optically active starting materials, stereoselective reagents such as chiral catalysts and auxiliaries well known in the art. Alternatively, racemic mixtures of such compounds may be separated or partially enriched using, for example, chromatographic methods with chiral stationary phases, chiral resolving agents, and the like. Diastereomers may be separated by physical methods such as chromatography or crystallization.

The methods presented in the schemes provided by the present disclosure are illustrative rather than comprehensive.

A compound of formula (I) or a pharmaceutically acceptable salt or solvate or hydrate thereof may be provided according to Scheme 1.

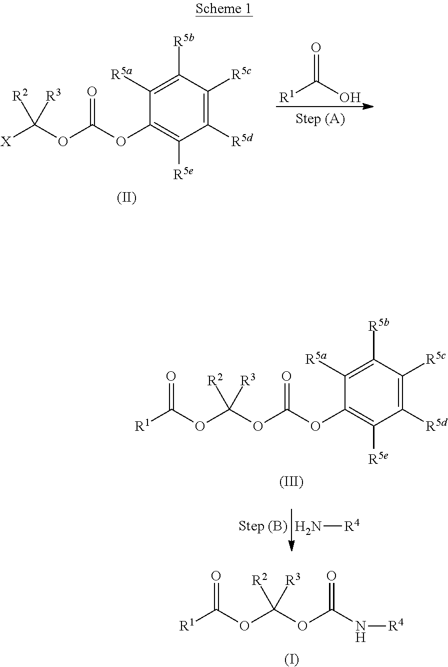

wherein X, R¹, R², R³, R⁴, R^{5a}, R^{5b}, R^{5c}, R^{5d}, and R^{5e} are as described herein.

### Representative Synthesis of Starting Carbonates of Formula (II)

The starting carbonate compounds of formula (II) may be prepared by reacting the appropriate phenol with the appropriate chloroformate as described below.

### Description 1-1

### 1-Chloroethyl 2-fluorophenyl carbonate (D1-1)

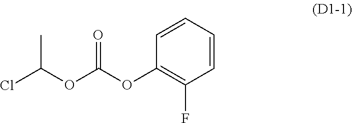

### Method A

2-Fluorophenol (4.87 g) and chloroethyl chloroformate (6.2 ml) were mixed in acetonitrile (25 ml) at 0° C. Triethylamine (7.9 ml) was slowly added while keeping the reaction temperature ≦10° C. The reaction mixture was stirred for approximately 5 minutes at ≦10° C., then warmed up to room temperature (˜22° C.). The reaction was stirred at ˜22° C. for one hour. The solid in the reaction was filtered and the filtrate was distilled to remove acetonitrile. The reaction mixture was then extracted with tert-butyl methyl ether (50 ml) and water (20 ml) to remove triethylamine hydrochloride salt. Distillation to remove solvents yielded the title compound (9.71 g).

### Method B

2-Fluorophenol (7.08 g) and chloroethyl chloroformate (13.90 g) were mixed in ethyl acetate (170 ml) under a nitrogen atmosphere. The mixture was cooled to −20° C. Triethylamine (9.7 ml) was slowly added to the reaction over 40 minutes while keeping temperature at ≦−15° C. The reaction was warmed to −5° C., and stirred for 1 hour. The solid in the reaction was filtered and washed with ethyl acetate (20 ml). The combined ethyl acetate solution was washed with water (50 ml) twice. Solvents and excess chloroethyl chloroformate were distilled off under reduced pressure to yield the title compound (14.1 g).

### Method C

To a 1 L jacketed laboratory reactor was charged water (700 ml), sodium bicarbonate (72.0 grams). The mixture was cooled to 0° C. A premixed solution containing 2-fluorophenol (60.0 grams) and chloroethyl chloroformate (80.3 grams) was slowly added to the reaction over 1 hour. The reaction was stirred at 0° C. for 2.5 hours. Heptane (400 ml) was added to the reaction. The reaction temperature was warmed up to 20° C. After 20 minutes stirring, the aqueous layer was removed. The heptane solution was washed with water (250 ml) twice. Heptane and excess chloroethyl chloroformate were distilled out at 30-40° C. under reduced pressure to yield the title compound as an oil (113.1 g).

### Description 1-2

### 1-Chloroethyl 3-fluorophenyl carbonate (D1-2)

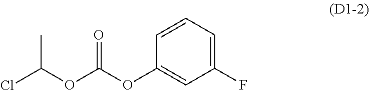

3-Fluorophenol (5.78 g) and dichloromethane (60 ml) were charged to reactor, followed by chloroethyl chloroformate (7.74 g). The reaction mixture was cooled to 0° C. Triethylamine (7.6 ml) was added slowly whilst maintaining the reaction temperature ≦20° C. The reaction mixture was warmed to room temperature, and stirred overnight. The reaction mixture was washed by water (50 ml) twice to remove triethylamine hydrochloride salt. Solvents and excess chloroethyl chloroformate were distilled off under reduced pressure to yield the title compound (10.7 g).

### Description 1-3

### 1-Chloroethyl 4-fluorophenyl carbonate (D1-3)

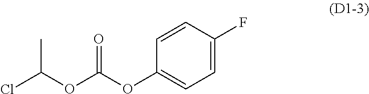

4-Fluorophenol (5.79 g) and dichloromethane (60 ml) were charged to reactor, followed by chloroethyl chloroformate (7.75 g). The reaction mixture was cooled to 0° C. Triethylamine (7.6 ml) was added slowly whilst maintaining the reaction temperature ≦20° C. The reaction mixture was warmed to room temperature, and stirred overnight. The reaction mixture was washed by water (40 ml) twice to remove triethylamine hydrochloride salt. Solvents and excess chloroethyl chloroformate were distilled off under reduced pressure to yield the title compound (11.1 g).

### Description 1-4

### 1-Chloroethyl 2,6-difluorophenyl carbonate (D1-4)

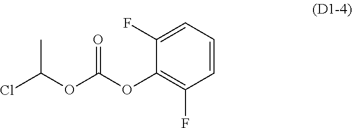

To a solution of 2,6-difluoro-phenol (8.7 g) in dichloromethane (80 ml) was added chloroethyl chloroformate (7.6 ml). The mixture was cooled to 0° C. Triethylamine (9.8 ml) was added slowly whilst maintaining the reaction temperature ≦20° C. The reaction was warmed up to room temperature and stirred for 1 hour. The reaction mixture was washed with water (60 ml) twice. Dichloromethane was distilled out to give a light oil product, which solidified to an off-white solid upon standing. Heptane (20 ml) was added to the crude product. After stiffing for 2 hours at 0° C., the slurry was filtered to yield the title compound as a white crystalline solid (14.8 g).

### Description 1-5

### 1-Chloroethyl 2-chlorophenyl carbonate (D1-5)

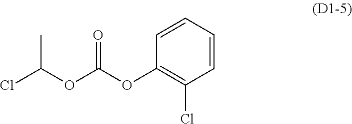

Water (4343.5 ml) and K₂CO₃ (1120.7 g) were charged to a 16 L jacketed laboratory reactor. The vessel contents were cooled to 0° C. A premixed solution of 2-chlorophenol (868.7 g) and 1-chloroethyl chloroformate (1159.3 g) in toluene (868.7 ml) were added to the vessel using a separate addition funnel over 52 minutes. The temperature of the vessel contents was maintained between −1° C. and 2.2° C. during the addition. After a further 2 hours 40 minutes (approx), the reaction was quenched by addition of aqueous ammonia hydroxide (28-30% as ammonia; 23 g). Toluene (5212.2 ml) was added and the mixture was stirred for 30 minutes. The aqueous layer was drained and the organic layer was washed in 2.6 L water.

The organic layer was maintained at 5° C. overnight. The organic layer was then distilled. The vessel contents were then washed with water, acetone and toluene. The mixture was concentrated on a rotovap at 50° C., then on a high vac overnight at room temperature to yield the title compound.

### Description 1-6

### 1-Chloroethyl 3-(methyloxy)phenyl carbonate (D1-6)

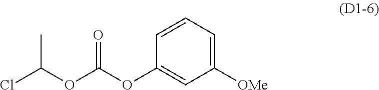

A solution of diethyl carbonate (0.93 vol) in N,N-diisopropylethylamine (1.47 vol, 1.05 eq) is prepared and cooled to 5° C. A solution of 3-methoxyphenol (1 wt, 1 eq) in 2M NaOH (4.23 vol, 1.05 eq) is prepared and cooled to 5° C. The diethyl carbonate and 3-methoxyphenol solutions are combined using a t-piece and fed directly into the loop of the loop reactor. 1-Chloroethylchloroformate (1.05 eq) is added via the centrifugal pump inlet to give a total residence time of 5-15 min. The outlet of the reactor is passed through a tube reactor (5-15 min residence time) held at 5° C. The process stream is separated in a phase separator or CLLE where the phases separate at ambient temperature with the organic phase at the top and the aqueous phase at the bottom.

The volumetric ratio of organic to aqueous is ˜2:3 at all times.

Percent yield range observed following the method of Description 1-6: >95%.

### Description 1-7

### 1-Chloroethyl 2-trifluoromethylphenyl carbonate (D1-7)

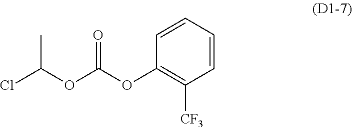 [SMILES: CC(Cl)OC(=O)Oc1ccccc1C(F)(F)F]

2-Trifluoromethylphenol (50 mmol) and chloroethyl chloroformate (6.2 ml) are mixed in acetonitrile (25 ml) at 0° C. Triethylamine (7.9 ml) is slowly charged while keeping the reaction temperature ≦10° C. The reaction mixture is stirred for approximately 5 minutes at ≦10° C., then warmed up to room temperature (˜22° C.). The reaction is stirred at ˜22° C. for one hour. The solid in the reaction is filtered and the filtrate is distilled to remove acetonitrile. The reaction mixture is then extracted with tert-butyl methyl ether (50 ml) and water (20 ml) to remove triethylamine hydrochloride salt. Distillation to remove solvents yields the title compound.

1-Chloroethyl 3-trifluoromethylphenyl carbonate (D1-8) and 1-chloroethyl 4-trifluoromethylphenyl carbonate (D1-9) can be prepared using the appropriate reagents and following the method described for D1-7.

### Description 1-10

### 1-Chloroethyl 4-methoxycarbonylphenyl carbonate (D1-10)

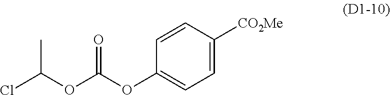 [SMILES: COC(=O)c1ccc(OC(=O)OC(C)Cl)cc1]

4-Hydroxybenzoic acid methyl ester (45 mmol) and chloroethyl chloroformate (6.2 ml) are mixed in acetonitrile (25 ml) at 0° C. Triethylamine (7.9 ml) is slowly charged while keeping the reaction temperature ≦10° C. The reaction mixture is stirred for approximately 5 minutes at ≦10° C., then warmed up to room temperature (˜22° C.). The reaction is stirred at ˜22° C. for one hour. The solid in the reaction is filtered and the filtrate is distilled to remove acetonitrile. The reaction mixture is then extracted with tert-butyl methyl ether (50 ml) and water (20 ml) to remove triethylamine hydrochloride salt. Distillation to remove solvents yields the title compound.

1-Chloroethyl 2-methoxycarbonylphenyl carbonate (D1-11) and 1-chloroethyl 3-methoxycarbonylphenyl carbonate (D1-12) can be prepared using the appropriate reagents and following the method described for D1-10.

### Description 1-13

### 1-Chloroethyl 3,4-methylenedioxyphenyl carbonate (D1-13)

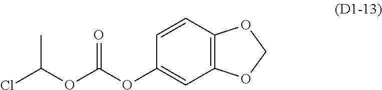 [SMILES: CC(Cl)OC(=O)Oc1ccc2c(c1)OCO2]

3,4-Methylenedioxyphenol (50 mmol) and chloroethyl chloroformate (6.0 ml) are mixed in acetonitrile (25 ml) at 0° C. Triethylamine (7.7 ml) is slowly charged while keeping the reaction temperature ≦10° C. The reaction mixture is stirred for approximately 5 minutes at ≦10° C., then warmed up to room temperature (˜22° C.). The reaction is stirred at ˜22° C. for one hour. The solid in the reaction is filtered and the filtrate is distilled to remove acetonitrile. The reaction mixture is then extracted with tert-butyl methyl ether (50 ml) and water (20 ml) to remove triethylamine hydrochloride salt. Distillation to remove solvents yields the title compound.

1-Chloroethyl 2,3-methylenedioxyphenyl carbonate (D1-14) can be prepared using the appropriate reagent and following the method described for D1-13.

### Description 1-15

### 1-Chloroethyl 3,4-ethylenedioxyphenyl carbonate (D1-15)

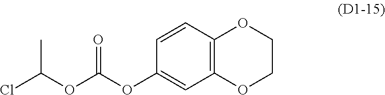 [SMILES: CC(Cl)OC(=O)Oc1ccc2c(c1)OCCO2]

3,4-Ethylenedioxyphenol (25 mmol) and chloroethyl chloroformate (3.0 ml) are mixed in acetonitrile (25 ml) at 0° C. Triethylamine (3.9 ml) is slowly charged while keeping the reaction temperature ≦10° C. The reaction mixture is stirred for approximately 5 minutes at ≦10° C., then warmed up to room temperature (˜22° C.). The reaction is stirred at ˜22° C. for one hour. The solid in the reaction is filtered and the filtrate is distilled to remove acetonitrile. The reaction mixture is then extracted with tert-butyl methyl ether (50 ml) and water (20 ml) to remove triethylamine hydrochloride salt. Distillation to remove solvents yields the title compound.

1-Chloroethyl 2,3-ethylenedioxyphenyl carbonate (D1-16) can be prepared using the appropriate reagent and following the method described for D1-15.

The following compounds can be prepared by following the method described above.

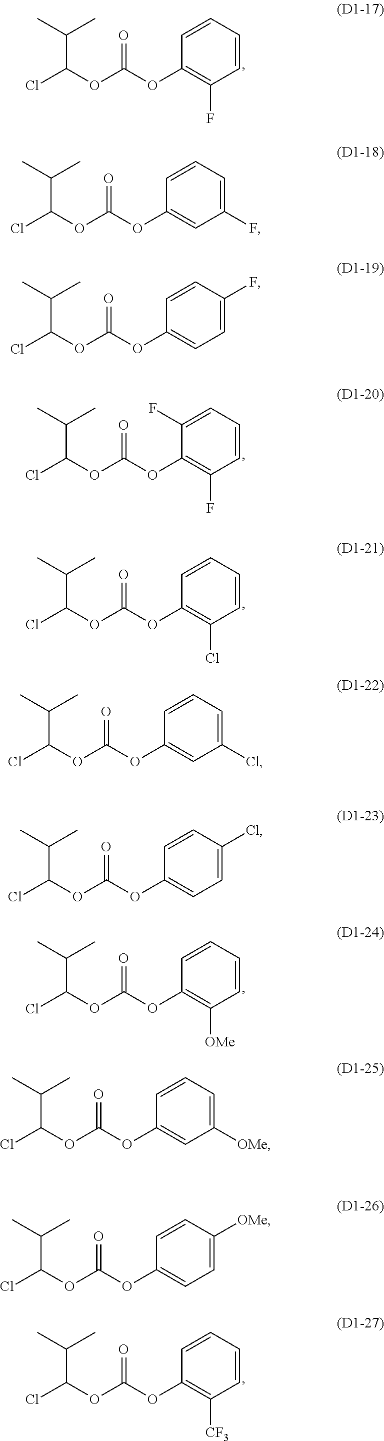

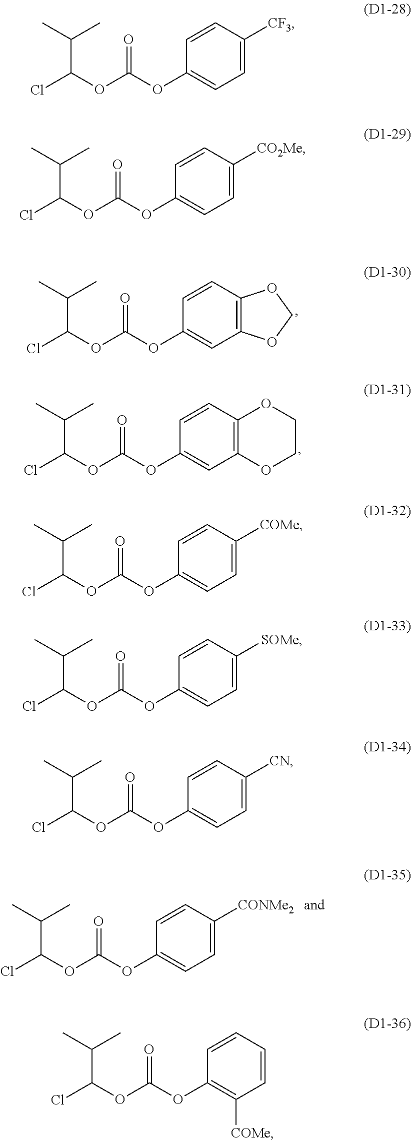

### Representative Synthesis of Carbonates of Formula (III) (Step A)

The carbonate compounds of formula (III) may be prepared by reacting the carbonate of formula (II) with the appropriate carboxylic acid as described below.

### Description 2-1

### 1-({[(2-Fluorophenyl)oxy]carbonyl}oxy)ethyl 2-methylpropanoate (D2-1)

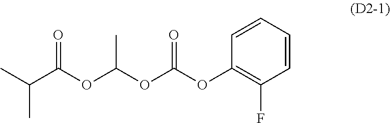 [SMILES: CC(OC(=O)Oc1ccccc1F)OC(=O)C(C)C]

### Method A

To a reaction vessel equipped with a Dean-Stark trap and reflux condenser was charged 1-chloroethyl 2-fluorophenyl carbonate (may be prepared as described in Description 1-1; 22.10 g), heptane (68 ml) and Cu₂O (10.80 g) under nitrogen atmosphere, followed by isobutyric acid (67 ml). The reactor was degassed by nitrogen. The reaction was heated to 115-120° C. and stirred for 3-5 hrs. The reaction mixture was then cooled to <50° C. The solids in the reaction were filtered and washed with heptane (80 ml). The filtrate and the heptane wash were combined. Water (100 ml) was added to the mixture. The mixture was cooled to 0° C. A dilute aqueous solution of ammonium hydroxide (10-15%, 90 ml) was slowly added to the mixture while keeping the temperature below 15° C. to adjust the pH of the mixture, which was adjusted to 9.5-10. After mixing for 10 minutes and settling for 10 minutes, the aqueous layer was removed. The organic layer was washed with water (80 ml). The solvents were distilled out to dryness to yield the title compound as an oil (24.3 g).

### Method B

To a mixture of 1-chloroethyl 2-fluorophenyl carbonate (may be prepared as described in Description 1-1; 0.58 g) and isobutyric acid (2.0 ml) was added silver isobutyrate (0.78 g). The mixture was heated to 90° C. for one hour. The reaction was then cooled to room temperature and filtered. The reactor and filter cake were washed with dichloromethane (10 ml). The combined filtrate and wash were distilled under reduced pressure until all isobutyric acid and solvents were removed to yield the title compound as an oil (0.58 g).

### Description 2-2

### 1-({[(3-Fluorophenyl)oxy]carbonyl}oxy)ethyl 2-methylpropanoate (D2-2)

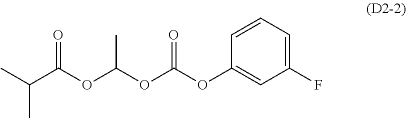 [SMILES: CC(OC(=O)Oc1cccc(F)c1)OC(=O)C(C)C]

To a reaction vessel under nitrogen atmosphere was charged 1-chloroethyl 3-fluorophenyl carbonate (may be prepared as described in Description 1-2; 21.8 g), isobutyric acid (80 ml) and Cu₂O (15.0 g). The reactor was degassed by nitrogen. The reaction was heated to 115-120° C. and stirred for 3-5 hrs. The reaction mixture was then was cooled to <50° C. The solids in the reaction were filtered and washed with heptane (160 ml). The filtrate and the heptane wash were combined. Water (100 ml) was added to the mixture. The mixture was cooled to 0° C. A dilute aqueous solution of ammonium hydroxide (˜15%) was slowly added to the mixture while keeping the temperature below 15° C. to adjust the pH of the mixture to 9.5-10. After mixing for 10 minutes and settling for 10 minutes, the aqueous layer was removed. The organic layer was separated and washed with water (110 ml). The solvents were distilled out to dryness to yield the title compound as an oil (20.3 g).

### Description 2-3

### 1-({[(4-Fluorophenyl)oxy]carbonyl}oxy)ethyl 2-methylpropanoate (D2-3)

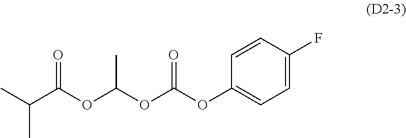 [SMILES: CC(OC(=O)Oc1ccc(F)cc1)OC(=O)C(C)C]

To a reaction vessel under nitrogen atmosphere was charged 1-chloroethyl 4-fluorophenyl carbonate (may be prepared as described in Description 1-3; 24.4 g), isobutyric acid (100 ml) and Cu₂O (16.8 g). The reactor was degassed by nitrogen. The reaction was heated to 100-110° C. and stirred for 4 hrs. When the reaction was complete as determined by HPLC, it was cooled to 20° C. The solids in the reaction were filtered and washed with heptane (100 ml). The filtrate and the heptane wash were combined. Water (100 ml) was added to the mixture. The mixture was cooled to 0° C. A dilute aqueous solution of ammonium hydroxide (−15%) was slowly added to the mixture while keeping the temperature below 15° C. to adjust the pH of the mixture, which was adjusted to 9.5-10. After mixing for 10 minutes and settling for 10 minutes, the aqueous layer was removed. The organic layer was washed with water (150 ml). The solvents were distilled out to dryness to yield the title compound as an oil (24.6 g).

### Description 2-4

### 1-({[(2,6-Difluorophenyl)oxy]carbonyl}oxy)ethyl 2-methylpropanoate (D2-4)

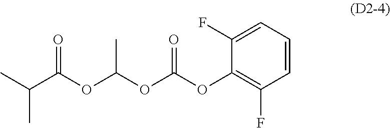 [SMILES: CC(OC(=O)Oc1c(F)cccc1F)OC(=O)C(C)C]

To a solution of 1-chloroethyl 2,6-difluorophenyl carbonate (may be prepared as described in Description 1-4; 0.1 g) in isobutyric acid (1.0 ml) was added silver isobutyrate (0.2 g). The mixture was heated to 90° C. for 3 hours. The reaction was then cooled to room temperature and filtered. The reactor and filter cake were washed with dichloromethane (10 ml). The combined filtrate and the wash were distilled under reduced pressure until all isobutyric acid and solvents were removed to yield the title compound as an oil (0.2 g).

### Description 2-5

### 1-({[(2-Chlorophenyl)oxy]carbonyl}oxy)ethyl 2-methylpropanoate (D2-5)

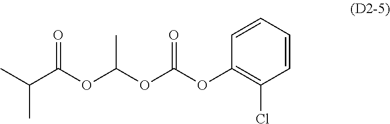 [SMILES: CC(OC(=O)Oc1ccccc1Cl)OC(=O)C(C)C]

Add Cu₂O, o-xylene, isobutyric acid, and isobutyric anhydride to a 1 L jacketed laboratory reactor sequentially in quick succession. Purge atmosphere immediately by pulling vacuum and backfilling with N₂ over three cycles. Heat the mixture to 120° C. and stir overnight at this temperature. Add 1-chloroethyl 2-chlorophenyl carbonate (may be prepared as described in Description 1-5) via syringe while maintaining the temperature of the reaction ≧105° C. Stir at 120° C. for 3 h or until HPLC indicates <0.5% PAR 1-chloroethyl 2-chlorophenyl carbonate. Cool the mixture to 20° C. and filter off the copper salt. Add o-xylene and water and neutralize the solution with NH₄OH (aq) to a final pH of 9.5-10. Drain off the aqueous layer and wash the organic layer with NH₄OH (aq). Drain off the aqueous layer and wash the organic layer with water. Pass through a polishing filter. Distill to minimum stir in jacketed laboratory reactor to yield the title compound (93%).

### Description 2-6

### 1-[({[3-(Methyloxy)phenyl]oxy}carbonyl)oxy]ethyl 2-methylpropanoate (D2-6)

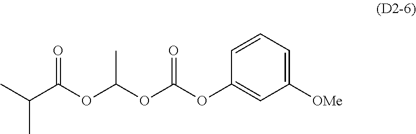 [SMILES: COc1cccc(OC(=O)OC(C)OC(=O)C(C)C)c1]

A solution of N,N-diisopropylethylamine (0.604 vol, 0.8 eq), iso-butyric acid (4.02 vol, 10 eq) and n-tetrabutylammonium bromide (0.14 wt, 0.1 eq) is heated to 105±5° C. before a solution of 1-chloroethyl 3-(methyloxy)phenyl carbonate (may be prepared as described in Description 1-6; 1.0 wt) is added. The resulting solution is heated at 105±5° C. for ca. 120 minutes or until the reaction is deemed complete by HPLC, before being cooled to 20±5° C.

The resulting reaction mixture is diluted with water (0.6 vol) before being diluted with tert-butyl methyl ether (8 vol). The resulting mixture is then washed with 5M NaOH (12 eq) counter currently through 3 stages in a CLLE at a rate of 3 g min⁻¹. The resulting organic phase is then washed with 0.5 M H₂SO₄ (1.9 eq) in the fourth stage of the CLLE before being washed with water (8 vol) in the final stage. The tert-butyl methyl ether phase is concentrated under atmospheric distillation to yield the title compound.

### Description 2-7

### 1-({[(2-Trifluoromethylphenyl)oxy]carbonyl}oxy)ethyl 2-methylpropanoate (D2-7)

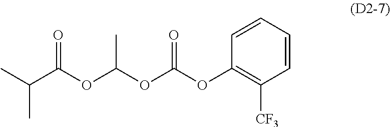 [SMILES: CC(OC(=O)Oc1ccccc1C(F)(F)F)OC(=O)C(C)C]

To a reaction vessel under nitrogen atmosphere is charged 1-chloroethyl 2-trifluoromethylphenyl carbonate (may be prepared as described in Description 1-7; 100 mmol), isobutyric acid (80 ml) and Cu₂O (15.0 g). The reactor is degassed by nitrogen. The reaction is heated to 115-120° C. and stirred for 3-5 hrs. The reaction mixture is then cooled to <50° C. The solids in the reaction are filtered and washed with heptane (160 ml). The filtrate and the heptane wash are combined. Water (100 ml) is added to the mixture. The mixture is cooled to 0° C. A dilute aqueous solution of ammonium hydroxide (˜15%) is slowly added to the mixture while keeping the temperature below 15° C. to adjust the pH of the mixture to 9.5-10. After mixing for 10 minutes and settling for 10 minutes, the aqueous layer is removed. The organic layer is washed with water (110 ml). The solvents are distilled out to dryness to yield the title compound.

### Description 2-8

### 1-({[(3-Trifluoromethylphenyl)oxy]carbonyl}oxy)ethyl 2-methylpropanoate (D2-8)

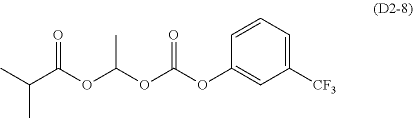 [SMILES: CC(OC(=O)Oc1cccc(C(F)(F)F)c1)OC(=O)C(C)C]

To a reaction vessel under nitrogen atmosphere is charged 1-chloroethyl 3-trifluoromethylphenyl carbonate (may be prepared as described in Description 1-8; 100 mmol), isobutyric acid (80 ml) and Cu₂O (15.0 g). The reactor is degassed by nitrogen. The reaction is heated to 115-120° C. and stirred for 3-5 hrs. The reaction mixture is then cooled to <50° C. The solids in the reaction are filtered and washed with heptane (160 ml). The filtrate and the heptane wash are combined. Water (100 ml) is added to the mixture. The mixture is cooled to 0° C. A dilute aqueous solution of ammonium hydroxide (˜15%) is slowly added to the mixture while keeping the temperature below 15° C. to adjust the pH of the mixture to 9.5-10. After mixing for 10 minutes and settling for 10 minutes, the aqueous layer is removed. The organic layer is washed with water (110 ml). The solvents are distilled out to dryness to yield the title compound.

### Description 2-9

### 1-({[(3-Trifluoromethylphenyl)oxy]carbonyl}oxy)ethyl 2-methylpropanoate (D2-9)

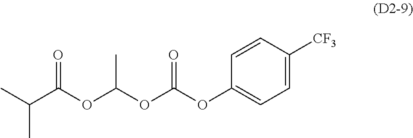 [SMILES: CC(OC(=O)Oc1ccc(C(F)(F)F)cc1)OC(=O)C(C)C]

To a reaction vessel under nitrogen atmosphere is charged 1-chloroethyl 2-trifluoromethylphenyl carbonate (may be prepared as described in Description 1-9; 100 mmol), isobutyric acid (80 ml) and Cu₂O (15.0 g). The reactor is degassed by nitrogen. The reaction is heated to 115-120° C. and stirred for 3-5 hrs. The reaction mixture is then cooled to <50° C. The solids in the reaction are filtered and washed with heptane (160 ml). The filtrate and the heptane wash are combined. Water (100 ml) is added to the mixture. The mixture is cooled to 0° C. A dilute aqueous solution of ammonium hydroxide (˜15%) is slowly added to the mixture while keeping the temperature below 15° C. to adjust the pH of the mixture to 9.5-10. After mixing for 10 minutes and settling for 10 minutes, the aqueous layer is removed. The organic layer is washed with water (110 ml). The solvents are distilled out to dryness to yield the title compound.

### Description 2-10

### 1-({[(4-Methoxycarbonylphenyl)oxy]carbonyl}oxy)ethyl 2-methylpropanoate (D2-10)

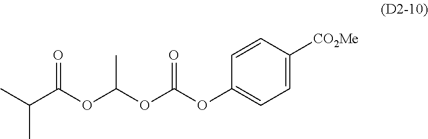 [SMILES: COC(=O)c1ccc(OC(=O)OC(C)OC(=O)C(C)C)cc1]

To a reaction vessel under nitrogen atmosphere is charged 1-chloroethyl 4-methoxycarbonylphenyl carbonate (may be prepared as described in Description 1-10; 100 mmol), isobutyric acid (80 ml) and Cu₂O (15.0 g). The reactor is degassed by nitrogen. The reaction is heated to 115-120° C. and stirred for 3-5 hrs. The reaction mixture is then cooled to <50° C. The solids in the reaction are filtered and washed with heptane (160 ml). The filtrate and the heptane wash are combined. Water (100 ml) is added to the mixture. The mixture is cooled to 0° C. A dilute aqueous solution of ammonium hydroxide (˜15%) is slowly added to the mixture while keeping the temperature below 15° C. to adjust the pH of the mixture to 9.5-10. After mixing for 10 minutes and settling for 10 minutes, the aqueous layer is removed. The organic layer is washed with water (110 ml). The solvents are distilled out to dryness to yield the title compound.

1-({[(2-Methoxycarbonylphenyl)oxy]carbonyl}oxy)ethyl 2-methylpropanoate (D2-11) and 1-({[(3-Methoxycarbonylphenyl)oxy]carbonyl}oxy)ethyl 2-methylpropanoate (D2-10) (D2-12) can be prepared using the appropriate reagents and following the method described for D2-10.

### Description 2-13

### 1-({[(3,4-Methylenedioxyphenyl)oxy]carbonyl}oxy)ethyl 2-methylpropanoate (D2-13)

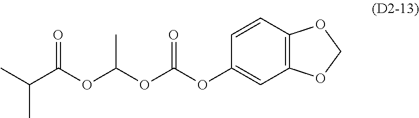 [SMILES: CC(OC(=O)Oc1ccc2c(c1)OCO2)OC(=O)C(C)C]

To a reaction vessel under nitrogen atmosphere is charged 1-chloroethyl 3,4-methylenedioxyphenyl carbonate (may be prepared as described in Description 1-13; 100 mmol), isobutyric acid (80 ml) and Cu₂O (15.0 g). The reactor is degassed by nitrogen. The reaction is heated to 115-120° C. and stirred for 3-5 hrs. The reaction mixture is then cooled to <50° C. The solids in the reaction are filtered and washed with heptane (160 ml). The filtrate and the heptane wash are combined. Water (100 ml) is added to the mixture. The mixture is cooled to 0° C. A dilute aqueous solution of ammonium hydroxide (˜15%) is slowly added to the mixture while keeping the temperature below 15° C. to adjust the pH of the mixture to 9.5-10. After mixing for 10 minutes and settling for 10 minutes, the aqueous layer is removed. The organic layer is washed with water (110 ml). The solvents are distilled out to dryness to yield the title compound.

1-({[(2,3-methylenedioxyphenyl)oxy]carbonyl}oxy)ethyl 2-methylpropanoate (D2-14) can be prepared using the appropriate reagents and following the method described for D2-13.

### Description 2-15

### 1-({[(3,4-Methylenedioxyphenyl)oxy]carbonyl}oxy)ethyl 2-methylpropanoate (D2-15)

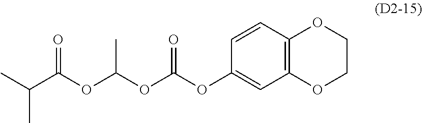 [SMILES: CC(OC(=O)Oc1ccc2c(c1)OCCO2)OC(=O)C(C)C]

To a reaction vessel under nitrogen atmosphere is charged 1-chloroethyl 3,4-ethylenedioxyphenyl carbonate (may be prepared as described in Description 1-13; 100 mmol), isobutyric acid (80 ml) and Cu₂O (15.0 g). The reactor is degassed by nitrogen. The reaction is heated to 115-120° C. and stirred for 3-5 hrs. The reaction mixture is then cooled to <50° C. The solids in the reaction are filtered and washed with heptane (160 ml). The filtrate and the heptane wash are combined. Water (100 ml) is added to the mixture. The mixture is cooled to 0° C. A dilute aqueous solution of ammonium hydroxide (˜15%) is slowly added to the mixture while keeping the temperature below 15° C. to adjust the pH of the mixture to 9.5-10. After mixing for 10 minutes and settling for 10 minutes, the aqueous layer is removed. The organic layer is washed with water (110 ml). The solvents are distilled out to dryness to yield the title compound.

1-({[(2,3-ethylenedioxyphenyl)oxy]carbonyl}oxy)ethyl 2-methylpropanoate (D2-16) can be prepared using the appropriate reagents and following the method described for compound D2-15.

The following compounds can be prepared by following the methods described above.

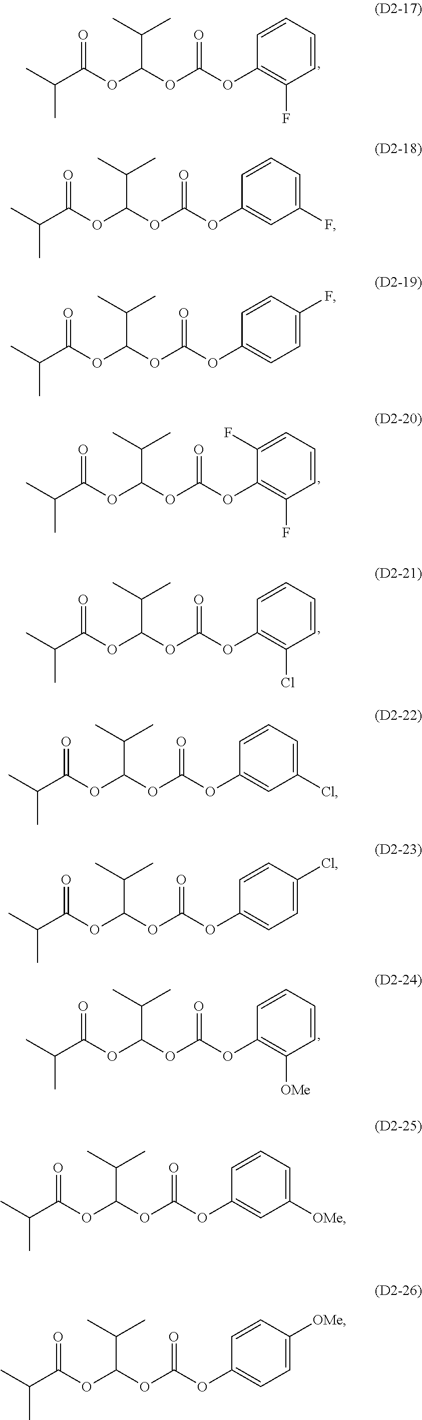

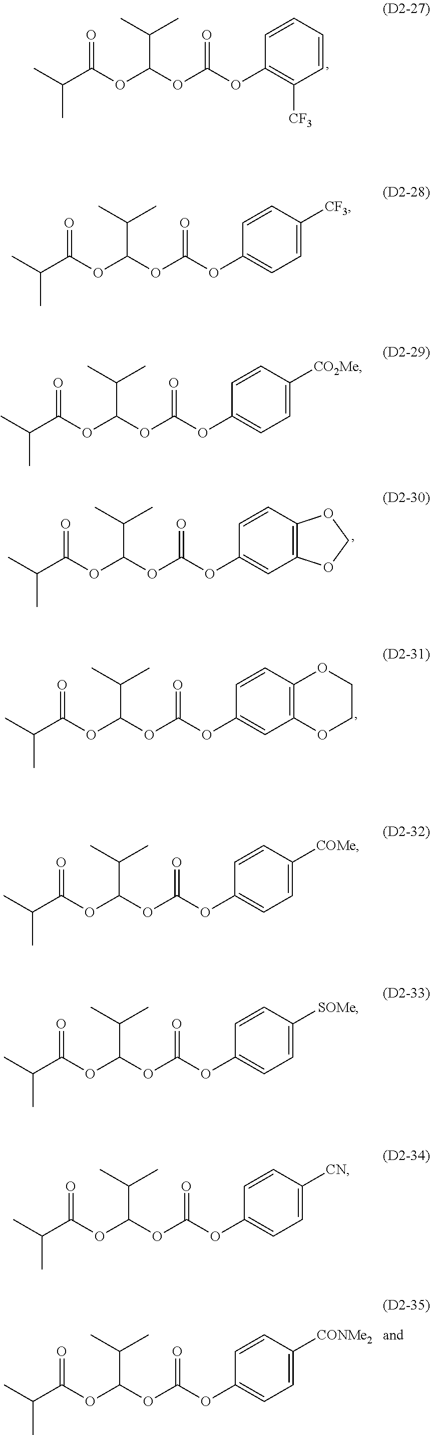

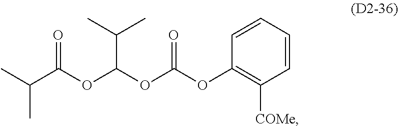

### Representative Preparation of Pure Isomers of Carbonates of Formula (III)

The pure enantiomers of carbonate compounds of formula (III) may be prepared by enzymatic methods or other conventional methods known to one skilled in the art. For example, the R-isomer of the carbonate may be prepared by enzymatic reaction of, or resolution of, a carbonate of formula (III) with lipase or other suitable enzymes as described in U.S. Pat. No. 7,872,046 or in U.S. Pat. No. 8,062,870.

### Description 3-1

### 1(R)-({[(2-Fluorophenyl)oxy]carbonyl}oxy)ethyl 2-methylpropanoate (D3-1)

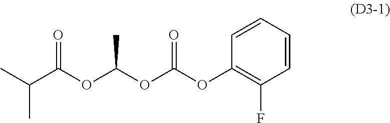 [SMILES: CC(C)C(=O)O[C@@H](C)OC(=O)Oc1ccccc1F]

1-({[(2-Fluorophenyl)oxy]carbonyl}oxy)ethyl 2-methylpropanoate (D2-1), prepared as described above (200 g) and lipase from Candida antarctica, immobilized on acrylic resin, (8.0 g) is stirred in phosphate buffered saline, pH 7.2, (1.6 L) at room temperature. The progress of the reaction is monitored by ¹H-NMR using the chiral solvating agent [(R)-(+)-2,2,2-trifluoro-1-(9-anthryl)ethanol] and is complete within 8-20 h. The reaction mixture is diluted with diethyl ether and the diethyl ether layer separated and filtered through a pad of Celite to remove the enzyme. The ether phase is washed repeatedly with water then brine, and dried over anhydrous Na₂SO₄. Removal of the solvent in vacuo affords the title compound (D3-1).

The following compounds can be prepared by following the method described for compound D3-1 and using the appropriate starting materials.

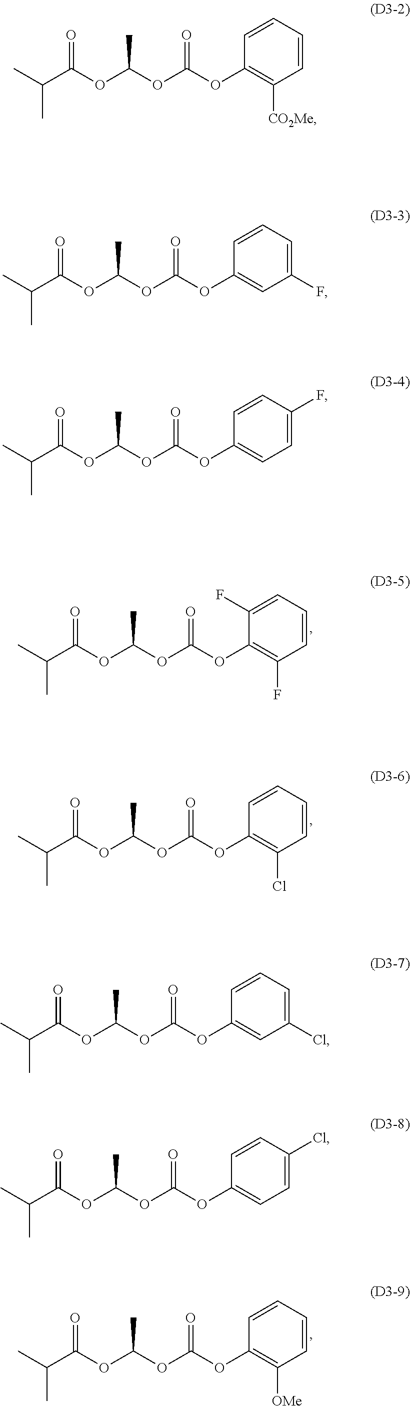

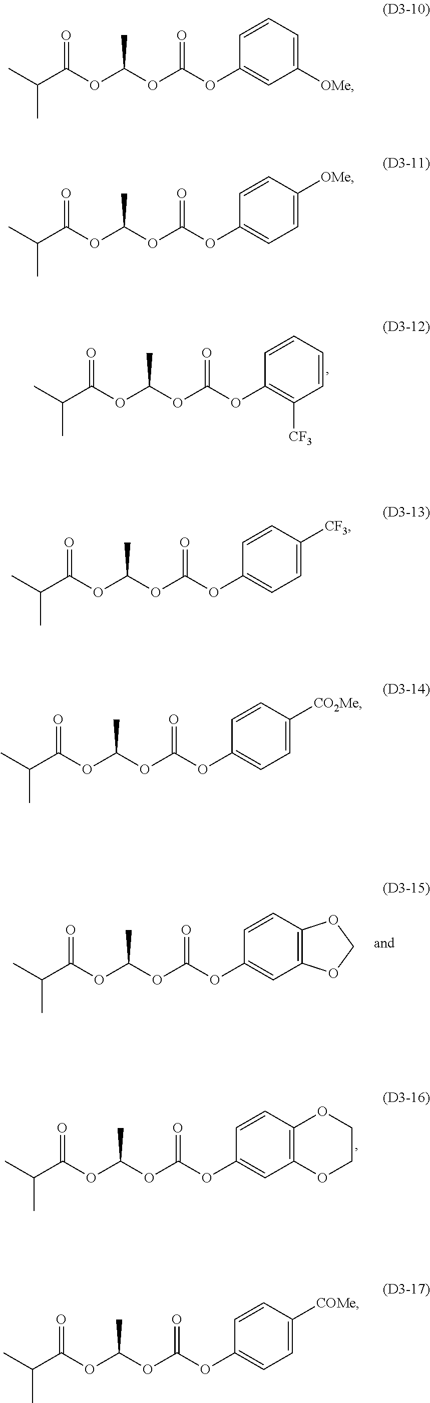

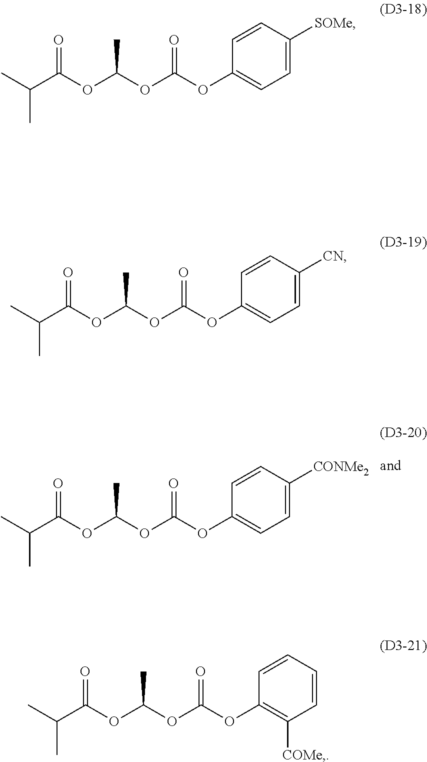

### Description 3-22

### 1(S)-({[(2-Fluorophenyl)oxy]carbonyl}oxy)-2-methylpropyl 2-methylpropanoate (D3-22)

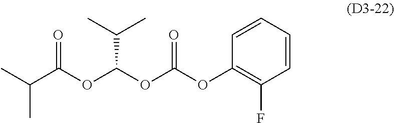 [SMILES: CC(C)C(=O)O[C@@H](OC(=O)Oc1ccccc1F)C(C)C]

A suspension of enzyme (5-10% by weight) in 50 mM pH 7.2 phosphate buffer (45 mL) and the racemic carbonate (D2-17) (10 mmol) in isopropyl ether (5 mL) is shaken on an orbital shaker at room temperature (25° C.). The reaction is monitored by ¹H-NMR using chiral solvating agent. After the reaction is complete the reaction mixture is filtered through a pad of CELITE® 545, followed by extraction with methyl-tert-butyl ether (MTBE). The organic layer is separated, washed with water and brine, and dried over anhydrous sodium sulfate (Na₂SO₄). The removal of the solvents under vacuo yields the corresponding enzymatically resolved D3-17 carbonate.

The following compounds can be prepared by following the method described for compound D3-22 and using the appropriate starting materials.

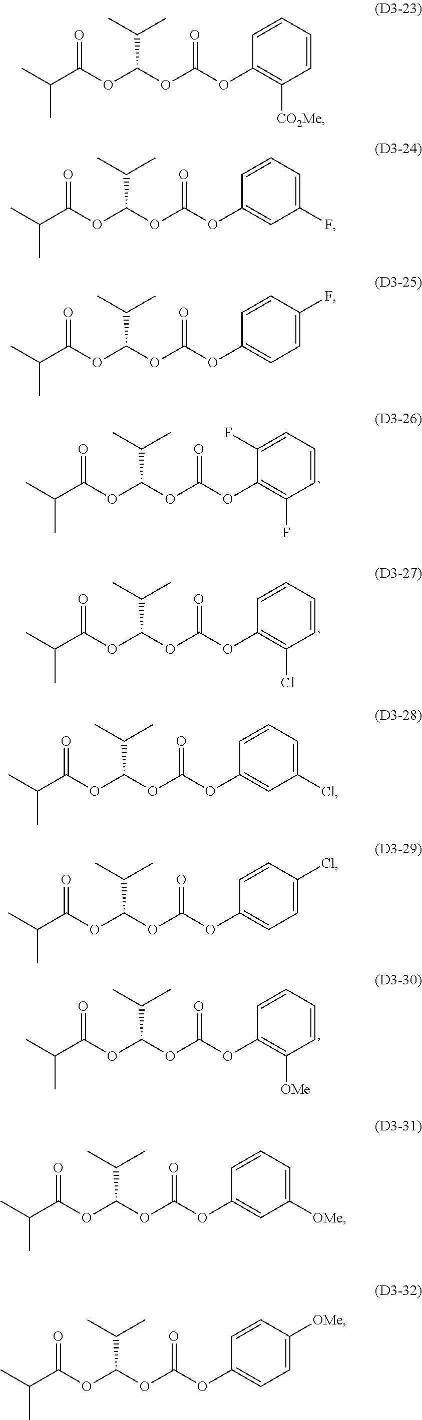

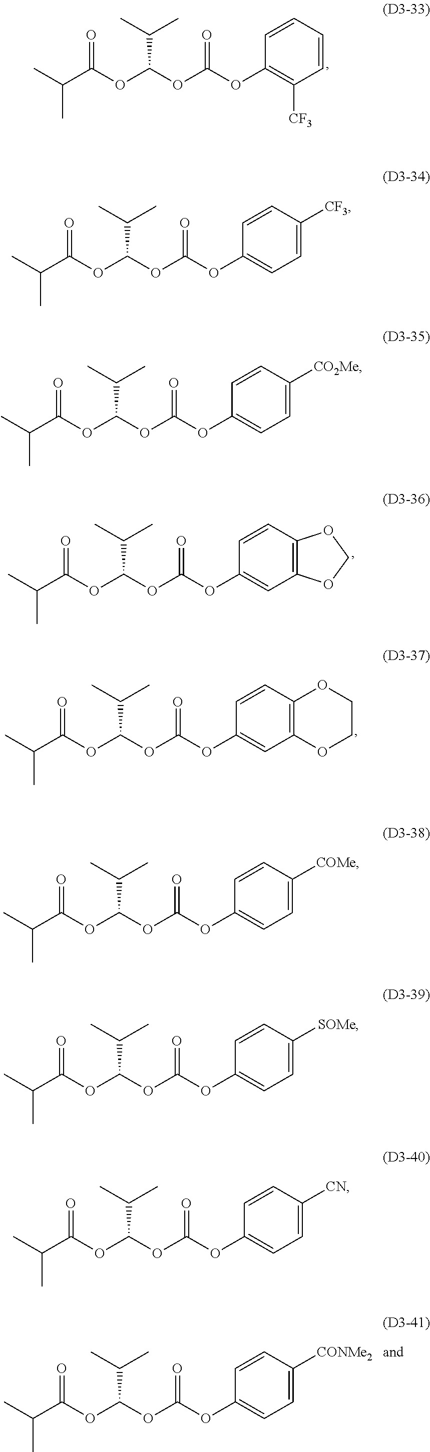

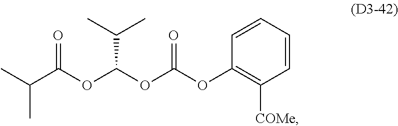

### Representative Synthesis of Compounds of Formula (I) (Step B)

The carbamate compounds of formula (I) may be prepared by reacting the carbonate of formula (III) with the appropriate amine as described below.

### Example 1

### 1-{[(α-Isobutanoyloxyethoxy)carbonyl]aminomethyl}-1-cyclohexane acetic acid (E1)

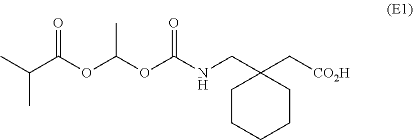 [SMILES: CC(OC(=O)NCC1(CC(=O)O)CCCCC1)OC(=O)C(C)C]

### Method A

To a jacketed laboratory reactor was charged 1-({[(2-fluorophenyl)oxy]carbonyl}oxy)ethyl 2-methylpropanoate (may be prepared as described in Description 2-1; 20.0 g) in heptanes solution (40 ml). Gabapentin (13.94 g), water (40 ml) and acetonitrile (30 ml) were charged and stirred for 10 minutes at 10-20° C. Triethylamine (11.4 ml) was charged over 5 minutes. The reaction was stirred at 10-20° C. for 4-8 hrs until 1-({[(2-fluorophenyl)oxy]carbonyl}oxy)ethyl 2-methylpropanoate was <2% by HPLC. 2 M H₂SO₄ aqueous solution (20 ml) was charged to acidify the reaction to pH 4-4.5. Tert-butyl methyl ether (90 ml) was charged and mixed for ˜10 minutes. After removal of aq. layer, the organic layer was washed with water (30 ml). The solvents were stripped off under reduced pressure with process temperature ≦35° C. and reactor jacket temperature ≦45° C. Heptane (100 ml) was charged and stripped off under vacuum. The crude product was used in crystallization directly.

Heptane (180 ml) and tert-butyl methyl ether (20 ml) were charged to the crude product. The mixture was then warmed up to 40-45° C. to achieve a clear solution. The process temperature was cooled to ˜30° C. over ˜50 minutes. A 3% w/w seed crystal was added. The temperature was slowly cooled to 15° C. and stayed at 15° C. for ˜6 hrs. When a significant amount of the product solid came out of solution (monitored by React-IR), the mixture was slowly cooled to −2 to 0° C. After holding at −2 to 0° C. for 9 hrs, the mixture was filtered and washed w/ heptane (60 ml) and heptane/tert-butyl methyl ether (10:1) (60 ml). The wet cake was dried at 25-30° C. under high vacuum overnight. A white crystalline product (17.08 grams, 80% yield) was obtained.

### Method B

To a jacketed laboratory reactor was charged 1-({[(3-fluorophenyl)oxy]carbonyl}oxy)ethyl 2-methylpropanoate (may be prepared as described in Description 2-2; 10.06 g) and tert-butyl methyl ether (20 ml). Gabapentin (7.65 g), water (20 ml) and acetonitrile (15 ml) were charged and stirred for ˜10 minutes at 10-20° C. Triethylamine (6.2 ml) was charged over ˜5 minutes. The reaction was stirred at 10-20° C. for 5 hrs. 10% potassium hydrogensulfate aqueous solution (67 ml) was charged to acidify reaction to pH 4-4.5. Tert-butyl methyl ether (80 ml) was charged and mixed for ˜10 minutes. After removal of aq. layer, the organic layer was washed with water (50 ml). The solvents were stripped off under reduced pressure with process temperature ≦35° C. and reactor jacket temperature ≦45° C. Methylcyclohexane (50 ml) was charged and stripped off under vacuum. The crude product was used in crystallization directly.

Heptane (92 ml) and tert-butyl methyl ether (10 ml) were charged to the crude product. The mixture was then warmed up to 40-45° C. to achieve a clear solution. The process temperature was cooled to ˜30° C. over ˜50 minutes. A 3% w/w seed crystal was added. The temperature was slowly cooled to 15° C. and stayed at 15° C. for ˜6 hrs. When a significant amount of the product solid came out of solution (monitored by React-IR), the mixture was slowly cooled to −2 to 0° C. After holding at −2 to 0° C. for ˜9 hrs, the mixture was filtered and washed w/ heptane (40 ml) and heptane/tert-butyl methyl ether (10:1) (30 ml). The wet cake was dried at 25-30° C. under high vacuum overnight. A white crystalline product (10.6 grams, 83% yield) was obtained.

### Method C

To a jacketed laboratory reactor was charged 1-({[(4-fluorophenyl)oxy]carbonyl}oxy)ethyl 2-methylpropanoate (may be prepared as described in Description 2-3; 10.81 g) and tert-butyl methyl ether (23 ml). Gabapentin (8.2 g), water (20 ml) and acetonitrile (20 ml) were charged and stirred for 10 minutes at 20° C. Triethylamine (6.7 ml) was charged over 5 minutes. The reaction was stirred at 20° C. for 6 hrs. 10% potassium hydrogensulfate aqueous solution (67 ml) was charged to acidify reaction to pH 4-4.5. Tert-butyl methyl ether (80 ml) was charged and mixed for 10 minutes. After removal of aq. layer, the organic layer was washed with water (35 ml). The solvents were stripped off under reduced pressure with process temperature ≦35° C. and reactor jacket temperature ≦45° C. Heptane (60 ml) was charged and stripped off under vacuum. The crude product was used in crystallization directly.

Heptane (100 ml) and tert-butyl methyl ether (14 ml) were charged to the crude product. The mixture was then warmed up to 40-45° C. to achieve a clear solution. The process temperature was cooled to ˜30° C. over ˜50 minutes. A 3% w/w seed crystal was added. The temperature was slowly cooled to 15° C. and stayed at 15° C. for ˜6 hrs. When a significant amount of the product solid came out of solution (monitored by React-IR), the mixture was slowly cooled to −2 to 0° C. After holding at −2 to 0° C. for ˜9 hrs, the mixture was filtered and washed w/ heptane (40 ml) and heptane/tert-butyl methyl ether (10:1) (30 ml). The wet cake was dried at 25-30° C. under high vacuum overnight. A white crystalline product (10.6 grams, 83% yield) was obtained.

### Method D

To a reaction vessel was charged 1-({[(2,6-difluorophenyl)oxy]carbonyl}oxy)ethyl 2-methylpropanoate (may be prepared as described in Description 2-4; 0.10 g) and gabapentin (0.15 g) in acetonitrile (0.6 ml) and water (0.2 ml). The reaction was warmed to 60° C. and stirred for 1-2 hrs. The desired product was confirmed by LC-MS.

### Method E

Add gabapentin, 1-({[(2-chlorophenyl)oxy]carbonyl}oxy)ethyl 2-methylpropanoate (may be prepared as described in Description 2-5), and tetrahydrofuran to a 500 ml jacketed laboratory reactor. Cool suspension to 15° C. Add NaOH (aq), keeping the reaction temperature ≦30° C. Stir resulting solution at 30° C. for 0.5 h or until HPLC indicates <0.5% PAR 1-({[(2-chlorophenyl)oxy]carbonyl}oxy)ethyl 2-methylpropanoate. Add H₂SO₄ (aq), H₂O, and toluene, keeping the temperature of the reaction ≦30° C. Cool to 20° C. and separate layers. Extract aqueous layer with toluene. To aqueous layer, add H₂SO₄ (aq) and tert-butyl methyl ether, keeping the temperature of the reaction ≦25° C. Separate layers and wash organic layer with H₂O. Separate layers and add methylcyclohexane to organic layer. Distill at 100 torr, keeping the temperature of the reaction ≦40° C. Add methylcyclohexane and distill at 70 torr, keeping the temperature of the reaction ≦40° C. Adjust temperature to 35° C. and add tert-butyl methyl ether. Add methylcyclohexane and cool to 28° C. Seed with seed crystals of the title compound and hold at 28° C. for 5 h. Cool to 15° C. at 0.33° C./min and then hold at 15° C. for 1 h. Heat to 28° C. at 0.33° C./min and then hold at 28° C. for 1 h. Cool to 0° C. at 0.33° C./min and then hold at 0° C. for 1 h. Filter, washing the cake twice with cold methylcyclohexane. Dry under vacuum at 35° C.

Percent yield observed following the method of Example 1, method E: approximately 74%

### Method F

Gabapentin is dissolved in 4M sodium hydroxide to give solution A. 1-({[(2-chlorophenyl)oxy]carbonyl}oxy)ethyl 2-methylpropanoate (may be prepared as described in Description 2-5) is dissolved in tetrahydrofuran to give solution B. Solution A is combined with additional 4M sodium hydroxide in flow mode before being combined with solution B at 30° C. in flow mode in a reactor (residence time ca 1.5 hr).

The reaction mixture is combined with 2M sulphuric acid and additional water in flow mode, before a counter-current extraction with toluene is performed in continuous liquid-liquid extraction apparatus (typically across three stages). The heavy (aqueous) phase is combined with 2M sulfuric acid in flow mode, before a counter-current extraction with tetrahydrofuran and methylcyclohexane is performed in continuous liquid-liquid extraction apparatus (typically a single stage). The light (organic) phase is washed in a counter-current extraction with water in continuous liquid-liquid extraction apparatus (typically a single stage).

The organic (light) phase is combined with methylcyclohexane and tetrahydrofuran and water are removed by continuous reduced-pressure distillation at ca 57° C., 230 mbar.

A continuous cooling crystallisation is performed across (typically) two stirred-tank crystallisation vessels, with wet milling in the first reactor to promote nucleation and control particle size. The first crystallisation vessel is maintained at ca 32° C., with an average residence time of ca 2 hr. The second crystallisation vessel is maintained at ca 20° C., with an average residence time of ca 2 hr.

A batch filtration is performed in a filter dryer. The damp cake is washed with methylcyclohexane. The isolated drug substance is dried at 20° C. (either in nitrogen flow or under vacuum).

Percent yield observed following the method of Example 1, method F: ca 80% th

### Method G

1-[({[3-(Methoxy)phenyl]oxy]carbonyl}oxy)ethyl 2-methylpropanoate (may be prepared as described in Description 2-6; 1 wt.) is dissolved in tetrahydrofuran (5 vol). Gabapentin (0.75 wt., 1.24 eq.) is then dissolved in 4N NaOH (1.1 vol, 1.24 eq.) and tetramethylguanidine (0.22 vol., 0.495 eq.). The two feeds are then mixed together at 20±5° C. and stirred at this temp for ca. 60 mins. A further aliquot of 4N NaOH (0.44 vol) is added and the reaction mixed for at least 30 mins until the reaction is deemed complete by HPLC.

Tert-butyl methyl ether (6 vol) and 0.5M H₂SO₄ (2.67 vol) are then added, the phases allowed to separate and the aqueous phase is then washed with tert-butyl methyl ether (2×2.5 vol). The aqueous phase is then acidified by the addition of 0.5M H₂SO₄ (3.37 vol) and extracted by tert-butyl methyl ether (2×2.5 vol). The combined organic phases are then washed with 0.05M H₂SO₄ (5.0 vol) and then water (5.0 vol).

Methylcyclohexane (10 vol) is then added and the mixture distilled under vacuum (temperature ≦45° C., P˜0.1-0.2 bar) to remove the tert-butyl methyl ether. The mixture is then allowed to cool to 20±5° C. and the product isolated by filtration. The cake is then washed with methylcyclohexane (2×2 vol) and pulled dry. The solid is then oven dried under vacuum at 40° C.

Percent yield range observed following the method of Example 1, method G: 60-80% th solution yield.

### Method H

1-[({[2-Trifluoromethylphenyl]oxy}carbonyl)oxy]ethyl 2-methylpropanoate (may be prepared as described in Description 2-7; 1 wt.) is dissolved in tetrahydrofuran (5 vol). Gabapentin (0.75 wt., 1.24 eq.) is then dissolved in 4N NaOH (1.1 vol, 1.24 eq.) and tetramethylguanidine (0.22 vol., 0.495 eq.). The two feeds are then mixed together at 20±5° C. and stirred at this temp for ca. 60 mins A further aliquot of 4N NaOH (0.44 vol) is added and the reaction mixed for at least 30 mins until the reaction is deemed complete by HPLC.

Tert-butyl methyl ether (6 vol) and 0.5M H₂SO₄ (2.67 vol) are then added, the phases allowed to separate and the aqueous phase is then washed with tert-butyl methyl ether (2×2.5 vol). The aqueous phase is then acidified by the addition of 0.5M H₂SO₄ (3.37 vol) and extracted by tert-butyl methyl ether (2×2.5 vol). The combined organic phases are then washed with 0.05M H₂SO₄ (5.0 vol) and then water (5.0 vol).

Methylcyclohexane (10 vol) is then added and the mixture distilled under vacuum (temperature ≦45° C., P˜0.1-0.2 bar) to remove the tert-butyl methyl ether. The mixture is then allowed to cool to 20±5° C. and the product isolated by filtration. The cake is then washed with methylcyclohexane (2×2 vol) and pulled dry. The solid is then oven dried under vacuum at 40° C. to yield the title product.

### Method I

1-[({[4-Methoxycarbonylphenyl]oxy}carbonyl)oxy]ethyl 2-methylpropanoate (may be prepared as described in Description 2-10; 1 wt.) is dissolved in tetrahydrofuran (5 vol). Gabapentin (0.75 wt., 1.24 eq.) is then dissolved in 4N NaOH (1.1 vol, 1.24 eq.) and tetramethylguanidine (0.22 vol., 0.495 eq.). The two feeds are then mixed together at 20±5° C. and stirred at this temp for ca. 60 mins A further aliquot of 4N NaOH (0.44 vol) is added and the reaction mixed for at least 30 mins until the reaction is deemed complete by HPLC.

Tert-butyl methyl ether (6 vol) and 0.5M H₂SO₄ (2.67 vol) are then added, the phases allowed to separate and the aqueous phase is then washed with tert-butyl methyl ether (2×2.5 vol). The aqueous phase is then acidified by the addition of 0.5M H₂SO₄ (3.37 vol) and extracted by tert-butyl methyl ether (2×2.5 vol). The combined organic phases are then washed with 0.05M H₂SO₄ (5.0 vol) and then water (5.0 vol).

Methylcyclohexane (10 vol) is then added and the mixture distilled under vacuum (temperature ≦45° C., P˜0.1-0.2 bar) to remove the tert-butyl methyl ether. The mixture is then allowed to cool to 20±5° C. and the product isolated by filtration. The cake is then washed with methylcyclohexane (2×2 vol) and pulled dry. The solid is then oven dried under vacuum at 40° C. to yield the title product.

### Method J

1-[({[3,4-Methylenedioxyphenyl]oxy}carbonyl)oxy]ethyl 2-methylpropanoate (may be prepared as described in Description 2-13; 1 wt.) is dissolved in tetrahydrofuran (5 vol). Gabapentin (0.75 wt., 1.24 eq.) is then dissolved in 4N NaOH (1.1 vol, 1.24 eq.) and tetramethylguanidine (0.22 vol., 0.495 eq.). The two feeds are then mixed together at 20±5° C. and stirred at this temp for ca. 60 mins A further aliquot of 4N NaOH (0.44 vol) is added and the reaction mixed for at least 30 mins until the reaction is deemed complete by HPLC.

Tert-butyl methyl ether (6 vol) and 0.5M H₂SO₄ (2.67 vol) are then added, the phases allowed to separate and the aqueous phase is then washed with tert-butyl methyl ether (2×2.5 vol). The aqueous phase is then acidified by the addition of 0.5M H₂SO₄ (3.37 vol) and extracted by tert-butyl methyl ether (2×2.5 vol). The combined organic phases are then washed with 0.05M H₂SO₄ (5.0 vol) and then water (5.0 vol).

Methylcyclohexane (10 vol) is then added and the mixture distilled under vacuum (temperature ≦45° C., P˜0.1-0.2 bar) to remove the tert-butyl methyl ether. The mixture is then allowed to cool to 20±5° C. and the product isolated by filtration. The cake is then washed with methylcyclohexane (2×2 vol) and pulled dry. The solid is then oven dried under vacuum at 40° C. to yield the title product.

### Method K

1-[({[3,4-Ethylenedioxyphenyl]oxy}carbonyl)oxy]ethyl 2-methylpropanoate (may be prepared as described in Description 2-13; 1 wt.) is dissolved in tetrahydrofuran (5 vol). Gabapentin (0.75 wt., 1.24 eq.) is then dissolved in 4N NaOH (1.1 vol, 1.24 eq.) and tetramethylguanidine (0.22 vol., 0.495 eq.). The two feeds are then mixed together at 20±5° C. and stirred at this temp for ca. 60 mins A further aliquot of 4N NaOH (0.44 vol) is added and the reaction mixed for at least 30 mins until the reaction is deemed complete by HPLC.

Tert-butyl methyl ether (6 vol) and 0.5M H₂SO₄ (2.67 vol) are then added, the phases allowed to separate and the aqueous phase is then washed with tert-butyl methyl ether (2×2.5 vol). The aqueous phase is then acidified by the addition of 0.5M H₂SO₄ (3.37 vol) and extracted by tert-butyl methyl ether (2×2.5 vol). The combined organic phases are then washed with 0.05M H₂SO₄ (5.0 vol) and then water (5.0 vol).

Methylcyclohexane (10 vol) is then added and the mixture distilled under vacuum (temperature ≦45° C., P˜0.1-0.2 bar) to remove the tert-butyl methyl ether. The mixture is then allowed to cool to 20±5° C. and the product isolated by filtration. The cake is then washed with methylcyclohexane (2×2 vol) and pulled dry. The solid is then oven dried under vacuum at 40° C. to yield the title product.

### Example 2

### 3(S)-{[(α-Isobutanoyloxyethoxy)carbonyl]aminomethyl}-5-methylhexanoic acid (F1)

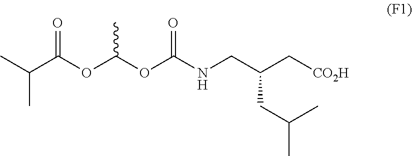 [SMILES: CC(C)C[C@H](CNC(=O)OC(C)OC(=O)C(C)C)CC(=O)O]

### Method A

To a jacketed laboratory reactor is charged 1-({[(2-fluorophenyl)oxy]carbonyl}oxy)ethyl 2-methylpropanoate (may be prepared as described in Description 2-1; 20.0 g) in heptanes solution (40 ml). Pregabalin (13.3 g), water (40 ml) and acetonitrile (30 ml) are charged and the mixture is stirred for 10 minutes at 10-20° C. Triethylamine (11.4 ml) is added over 5 minutes. The reaction is stirred at 10-20° C. for 4-8 hrs until 1-({[(2-fluorophenyl)oxy]carbonyl}oxy)ethyl 2-methylpropanoate is <2% by HPLC. 2 M H₂SO₄ aqueous solution (20 ml) is charged to acidify reaction to pH 4-4.5. Tert-butyl methyl ether (90 ml) is charged and mixed for ˜10 minutes. After removal of aq. layer, the organic layer is washed with water (30 ml). The solvents are stripped off under reduced pressure with process temperature ≦35° C. and reactor jacket temperature ≦45° C. to yield the title product.

### Method B

To a jacketed laboratory reactor is charged 1-({[(3-fluorophenyl)oxy]carbonyl}oxy)ethyl 2-methylpropanoate (may be prepared as described in Description 2-2; 10.06 g) and tert-butyl methyl ether (20 ml). Pregabalin (7.35 g), water (20 ml) and acetonitrile (15 ml) are added and the mixture is stirred for ˜10 minutes at 10-20° C. Triethylamine (6.2 ml) is charged over 5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for ˜10 minutes. After removal of aq. layer, the organic layer is washed with water (50 ml). The solvents are stripped off under reduced pressure with process temperature ≦35° C. and reactor jacket temperature ≦45° C. to yield the crude title product.

### Method C

To a jacketed laboratory reactor is charged 1-({[(4-trifluoromethylphenyl)oxy]carbonyl}oxy)ethyl 2-methylpropanoate (may be prepared as described in Description 2-9; 10.80 g) and tert-butyl methyl ether (20 ml). Pregabalin (7.35 g), water (20 ml) and acetonitrile (15 ml) are added and the mixture is stirred for ˜10 minutes at 10-20° C. Triethylamine (6.2 ml) is charged over ˜5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for ˜10 minutes. After removal of aq. layer, the organic layer is washed with water (50 ml). The solvents are stripped off under reduced pressure with process temperature ≦35° C. and reactor jacket temperature ≦45° C. to yield the crude title product.

### Method D

To a jacketed laboratory reactor is charged 1-({[(4-methoxycarbonylphenyl)oxy]carbonyl}oxy)ethyl 2-methylpropanoate (may be prepared as described in Description 2-10; 10.80 g) and tert-butyl methyl ether (20 ml). Pregabalin (7.35 g), water (20 ml) and acetonitrile (15 ml) are added and the mixture is stirred for ˜10 minutes at 10-20° C. Triethylamine (6.2 ml) is charged over ˜5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for ˜10 minutes. After removal of aq. layer, the organic layer is washed with water (50 ml). The solvents are stripped off under reduced pressure with process temperature ≦35° C. and reactor jacket temperature ≦45° C. to yield the crude title product.

### Method E

To a jacketed laboratory reactor is charged 1-({[(3,4-methylenedioxyphenyl)oxy]carbonyl}oxy)ethyl 2-methylpropanoate (may be prepared as described in Description 2-13; 10.50 g) and tert-butyl methyl ether (20 ml). Pregabalin (7.35 g), water (20 ml) and acetonitrile (15 ml) are added and the mixture is stirred for ˜10 minutes at 10-20° C. Triethylamine (6.2 ml) is charged over ˜5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for ˜10 minutes. After removal of aq. layer, the organic layer is washed with water (50 ml). The solvents are stripped off under reduced pressure with process temperature ≦35° C. and reactor jacket temperature ≦45° C. to yield the crude title product.

### Example 3

### 3(S)-{[(1R)-1-(2-methylpropanoyloxyethoxy)carbonyl]aminomethyl}-5-methylhexanoic acid (F2)

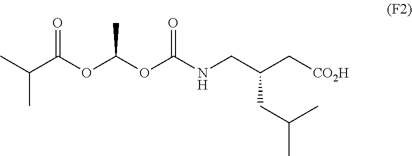 [SMILES: CC(C)C[C@H](CNC(=O)O[C@H](C)OC(=O)C(C)C)CC(=O)O]

### Method A

The compound is prepared following the method described in Example 2 (Method A) and reacting 1(R)-({[(2-fluorophenyl)oxy]carbonyl}oxy)ethyl 2-methylpropanoate (may be prepared as described in Description 3-1) and pregabalin.

### Method B

The compound is prepared following the method described in Example 2 (Method B) and reacting 1(R)-({[(3-fluorophenyl)oxy]carbonyl}oxy)ethyl 2-methylpropano ate (may be prepared as described in Description 3-3) and pregabalin.

### Method C

The compound is prepared following the method described in Example 2 (Method C) and reacting 1(R)-({[(4-trifluoromethylphenyl)oxy]carbonyl}oxy)ethyl 2-methylpropanoate (may be prepared as described in Description 3-13) and pregabalin.

### Method D

The compound is prepared following the method described in Example 2 (Method D) and reacting 1(R)-({[(4-methoxyphenyl)oxy]carbonyl}oxy)ethyl 2-methylpropanoate (may be prepared as described in Description 3-11) and pregabalin.

### Method E

The compound is prepared following the method described in Example 2 (Method E) and reacting 1(R)-({[(3,4-methylenedioxy phenyl)oxy]carbonyl}oxy)ethyl 2-methylpropanoate (may be prepared as described in Description 3-15 and pregabalin.

### Example 4

### 4-{[(α-Isobutanoyloxyl-β-methylpropoxy)carbonyl]amino}-3 (R)-(4-chlorophenyl)butanoic acid (G1)

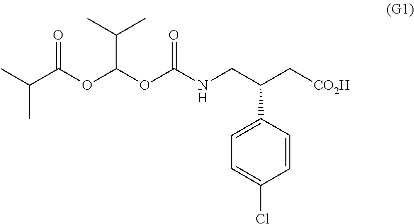 [SMILES: CC(C)C(=O)OC(OC(=O)NC[C@H](CC(=O)O)c1ccc(Cl)cc1)C(C)C]

### Method A

To a jacketed laboratory reactor is charged 1-({[(2-fluorophenyl)oxy]carbonyl}oxy)-2-methylproyl 2-methylpropanoate (may be prepared by following an analogous method described for D2-1 and using the appropriate reagents; 20.0 g) in heptanes solution (40 ml). R-Baclofen (14.75 g), water (40 ml) and acetonitrile (30 ml) are charged and the mixture is stirred for ˜10 minutes at 10-20° C. Triethylamine (11.4 ml) is added over ˜5 minutes. The reaction is stirred at 10-20° C. for 4-8 hrs until 1-({[(2-fluorophenyl)oxy]carbonyl}oxy)-2-methylpropyl 2-methylpropanoate is <2% by HPLC. 2 M H₂SO₄ aqueous solution (20 ml) is charged to acidify reaction to pH 4-4.5. Tert-butyl methyl ether (90 ml) is charged and mixed for ˜10 minutes. After removal of the aqueous layer, the organic layer is washed with water (30 ml). The solvents are stripped off under reduced pressure with process temperature ≦35° C. and reactor jacket temperature ≦45° C. to yield the title product.

### Method B

To a jacketed laboratory reactor is charged 1-({[(3-fluorophenyl)oxy]carbonyl}oxy)-2-methylpropyl 2-methylpropanoate (may be prepared by following an analogous method described for D2-2 and using the appropriate reagents; 12 g) and tert-butyl methyl ether (20 ml). R-Baclofen (7.35 g), water (20 ml) and acetonitrile (15 ml) are added and the mixture is stirred for ˜10 minutes at 10-20° C. Triethylamine (6.2 ml) is charged over ˜5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for ˜10 minutes. After removal of the aqueous layer, the organic layer is washed with water (50 ml). The solvents are stripped off under reduced pressure with process temperature ≦35° C. and reactor jacket temperature ≦45° C. to yield the crude title product.

### Method C

To a jacketed laboratory reactor is charged 1-({[(4-methoxycarbonylphenyl)oxy]carbonyl}oxy)-2-methylproyl 2-methylpropanoate (may be prepared by following an analogous method described for D2-10 and using the appropriate reagents; 20.0 g) in heptanes solution (40 ml). R-Baclofen (14.75 g), water (40 ml) and acetonitrile (30 ml) are charged and the mixture is stirred for ˜10 minutes at 10-20° C. Triethylamine (11.4 ml) is added over ˜5 minutes. The reaction is stirred at 10-20° C. for 4-8 hrs until 1-({[(2-fluorophenyl)oxy]carbonyl}oxy)-2-methylpropyl 2-methylpropanoate is <2% by HPLC. 2 M H₂SO₄ aqueous solution (20 ml) is charged to acidify reaction to pH 4-4.5. Tert-butyl methyl ether (90 ml) is charged and mixed for ˜10 minutes. After removal of the aqueous layer, the organic layer is washed with water (30 ml). The solvents are stripped off under reduced pressure with process temperature ≦35° C. and reactor jacket temperature ≦45° C. to yield the title product.

### Example 5

### 4-{[(1S)-2-methyl-1-(2-methylpropanoyloxy)carbonyl]amino}-3(R)-(4-chlorophenyl)butanoic acid (G2)

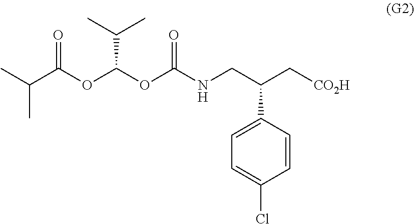 [SMILES: CC(C)C(=O)O[C@@H](OC(=O)NC[C@H](CC(=O)O)c1ccc(Cl)cc1)C(C)C]

### Method A

The compound is prepared following the method described in Example 4 (Method A) and reacting 1(S)-({[(2-fluorophenyl)oxy]carbonyl}oxy)-2-methylpropyl 2-methylpropanoate (may be prepared as described in Description 3-17) and R-baclofen.

### Method B

The compound is prepared following the method described in Example 4 (Method B) and reacting 1(S)-({[(3-fluorophenyl)oxy]carbonyl}oxy)-2-methylpropyl 2-methylpropanoate (may be prepared as described in Description 3-19) and R-baclofen.

### Method C

The compound is prepared following the method described in Example 4 (Method C) and reacting 1(S)-({[(4-methoxyphenyl)oxy]carbonyl}oxy)-2-methylpropyl 2-methylpropanoate (may be prepared as described in Description 3-27) and R-baclofen.

### Example 6

### 4-{[(α-Isobutanoyloxyethoxy)carbonyl]amino}-3(R)-(4-fluorophenyl)butanoic acid (H1)

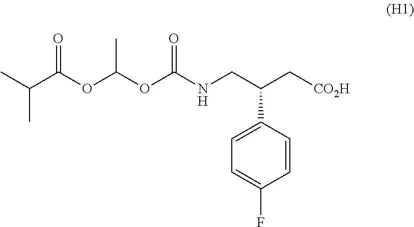 [SMILES: CC(OC(=O)NC[C@H](CC(=O)O)c1ccc(F)cc1)OC(=O)C(C)C]

### Method A

To a jacketed laboratory reactor is charged 1-({[(2-fluorophenyl)oxy]carbonyl}oxy)ethyl 2-methylpropanoate (may be prepared as described in Description 2-1; 20.0 g) in heptanes solution (40 ml). 4-Amino-3-(4-fluorophenyl)butanoic acid (14.00 g), water (40 ml) and acetonitrile (30 ml) are charged and the mixture is stirred for 10 minutes at 10-20° C. Triethylamine (11.4 ml) is added over ˜5 minutes. The reaction is stirred at 10-20° C. for 4-8 hrs until 1-({[(2-fluorophenyl)oxy]carbonyl}oxy)ethyl 2-methylpropanoate is <2% by HPLC. 2 M H₂SO₄ aqueous solution (20 ml) is charged to acidify reaction to pH 4-4.5. Tert-butyl methyl ether (90 ml) is charged and mixed for ˜10 minutes. After removal of aq. layer, the organic layer is washed with water (30 ml). The solvents are stripped off under reduced pressure with process temperature ≦35° C. and reactor jacket temperature ≦45° C. to yield the title product.

### Method B

To a jacketed laboratory reactor is charged 1-({[(3-fluorophenyl)oxy]carbonyl}oxy)ethyl 2-methylpropanoate (may be prepared as described in Description 2-2; 10.06 g) and tert-butyl methyl ether (20 ml). 4-Amino-3-(4-fluorophenyl)butanoic acid (8.30 g), water (20 ml) and acetonitrile (15 ml) are added and the mixture is stirred for ˜10 minutes at 10-20° C. Triethylamine (6.2 ml) is charged over 5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for ˜10 minutes. After removal of aq. layer, the organic layer is washed with water (50 ml). The solvents are stripped off under reduced pressure with process temperature ≦35° C. and reactor jacket temperature ≦45° C. to yield the crude title product.

### Method C

To a jacketed laboratory reactor is charged 1-({[(4-trifluoromethylphenyl)oxy]carbonyl}oxy)ethyl 2-methylpropanoate (may be prepared as described in Description 2-9; 10.80 g) and tert-butyl methyl ether (20 ml). 4-Amino-3-(4-fluorophenyl)butanoic acid (8.30 g), water (20 ml) and acetonitrile (15 ml) are added and the mixture is stirred for ˜10 minutes at 10-20° C. Triethylamine (6.2 ml) is charged over 5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for ˜10 minutes. After removal of aq. layer, the organic layer is washed with water (50 ml). The solvents are stripped off under reduced pressure with process temperature ≦35° C. and reactor jacket temperature ≦45° C. to yield the crude title product.

### Method D

To a jacketed laboratory reactor is charged 1-({[(4-methoxycarbonylphenyl)oxy]carbonyl}oxy)ethyl 2-methylpropanoate (may be prepared as described in Description 2-10; 10.80 g) and tert-butyl methyl ether (20 ml). 4-Amino-3-(4-fluorophenyl)butanoic acid (8.30 g), water (20 ml) and acetonitrile (15 ml) are added and the mixture is stirred for ˜10 minutes at 10-20° C. Triethylamine (6.2 ml) is charged over 5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for ˜10 minutes. After removal of aq. layer, the organic layer is washed with water (50 ml). The solvents are stripped off under reduced pressure with process temperature ≦35° C. and reactor jacket temperature ≦45° C. to yield the crude title product.

### Method E

To a jacketed laboratory reactor is charged 1-({[(3,4-methylenedioxyphenyl)oxy]carbonyl}oxy)ethyl 2-methylpropanoate (may be prepared as described in Description 2-13; 10.50 g) and tert-butyl methyl ether (20 ml). 4-Amino-3-(4-fluorophenyl)butanoic acid (8.30 g), water (20 ml) and acetonitrile (15 ml) are added and the mixture is stirred for ˜10 minutes at 10-20° C. Triethylamine (6.2 ml) is charged over 5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for ˜10 minutes. After removal of aq. layer, the organic layer is washed with water (50 ml). The solvents are stripped off under reduced pressure with process temperature ≦35° C. and reactor jacket temperature ≦45° C. to yield the crude title product.

### Example 7

### 4-{[(1R)-1-(1-(2-methylpropanoyloxy)ethoxy]carbonylamino}-3(R)-(4-fluorophenyl)butanoic acid (112)

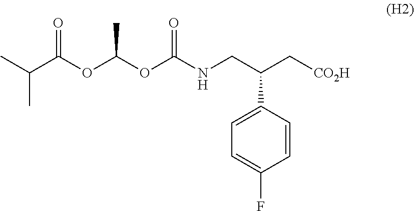 [SMILES: CC(C)C(=O)O[C@@H](C)OC(=O)NC[C@H](CC(=O)O)c1ccc(F)cc1]

### Method A

The compound is prepared following the method described in Example 6 (Method A) and reacting 1(R)-({[(2-fluorophenyl)oxy]carbonyl}oxy)ethyl 2-methylpropanoate (may be prepared as described in Description 3-1) and 4-amino-3-(4-fluorophenyl)butanoic acid.

### Method B

The compound is prepared following the method described in Example 6 (Method B) and reacting 1(R)-({[(3-fluorophenyl)oxy]carbonyl}oxy)ethyl 2-methylpropanoate (may be prepared as described in Description 3-3) and 4-amino-3-(4-fluorophenyl)butanoic acid.

### Method C

The compound is prepared following the method described in Example 6 (Method C) and reacting 1(R)-({[(4-trifluoromethylphenyl)oxy]carbonyl}oxy)ethyl 2-methylpropanoate (may be prepared as described in Description 3-13) and 4-amino-3-(4-fluorophenyl)butanoic acid.

### Method D

The compound is prepared following the method described in Example 6 (Method D) and reacting 1(R)-({[(4-methoxyphenyl)oxy]carbonyl}oxy)ethyl 2-methylpropanoate (may be prepared as described in Description 3-11) and 4-amino-3-(4-fluorophenyl)butanoic acid.

### Method E

The compound is prepared following the method described in Example 6 (Method E) and reacting 1(R)-({[(3,4-methylenedioxy phenyl)oxy]carbonyl}oxy)ethyl 2-methylpropanoate (may be prepared as described in Description 3-15) and 4-amino-3-(4-fluorophenyl)butanoic acid.

### Example 8

### 4-{[(α-Isobutanoyloxyethoxy)carbonyl]aminomethyl}-cyclohexanoic acid (J1)

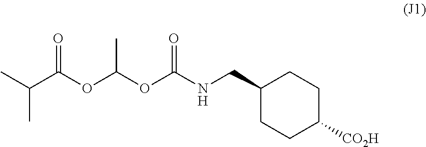 [SMILES: CC(OC(=O)NC[C@H]1CC[C@H](C(=O)O)CC1)OC(=O)C(C)C]

### Method A

To a jacketed laboratory reactor is charged 1-({[(2-fluorophenyl)oxy]carbonyl}oxy)ethyl 2-methylpropanoate (may be prepared as described in Description 2-1; 20.0 g) in heptanes solution (40 ml). Tranexamic acid (11.00 g), water (40 ml) and acetonitrile (30 ml) are charged and the mixture is stirred for ˜10 minutes at 10-20° C. Triethylamine (11.4 ml) is added over ˜5 minutes. The reaction is stirred at 10-20° C. for 4-8 hrs until 1-({[(2-fluorophenyl)oxy]carbonyl}oxy)ethyl 2-methylpropanoate is <2% by HPLC. 2 M H₂SO₄ aqueous solution (20 ml) is charged to acidify reaction to pH 4-4.5. Tert-butyl methyl ether (90 ml) is charged and mixed for ˜10 minutes. After removal of aq. layer, the organic layer is washed with water (30 ml). The solvents are stripped off under reduced pressure with process temperature ≦35° C. and reactor jacket temperature ≦45° C. to yield the title product.

### Method B

To a jacketed laboratory reactor is charged 1-({[(3-fluorophenyl)oxy]carbonyl}oxy)ethyl 2-methylpropanoate (may be prepared as described in Description 2-2; 10.06 g) and tert-butyl methyl ether (20 ml). Tranexamic acid (6.20 g), water (20 ml) and acetonitrile (15 ml) are added and the mixture is stirred for ˜10 minutes at 10-20° C. Triethylamine (6.2 ml) is charged over ˜5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for ˜10 minutes. After removal of aq. layer, the organic layer is washed with water (50 ml). The solvents are stripped off under reduced pressure with process temperature ≦35° C. and reactor jacket temperature ≦45° C. to yield the crude title product.

### Method C

To a jacketed laboratory reactor is charged 1-({[(4-trifluoromethylphenyl)oxy]carbonyl}oxy)ethyl 2-methylpropanoate (may be prepared as described in Description 2-9; 10.80 g) and tert-butyl methyl ether (20 ml). Tranexamic acid (7.10 g), water (20 ml) and acetonitrile (15 ml) are added and the mixture is stirred for 10 minutes at 10-20° C. Triethylamine (6.2 ml) is charged over ˜5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for 10 minutes. After removal of aq. layer, the organic layer is washed with water (50 ml). The solvents are stripped off under reduced pressure with process temperature ≦35° C. and reactor jacket temperature ≦45° C. to yield the crude title product.

### Method D

To a jacketed laboratory reactor is charged 1-({[(4-methoxycarbonylphenyl)oxy]carbonyl}oxy)ethyl 2-methylpropanoate (may be prepared as described in Description 2-10; 10.80 g) and tert-butyl methyl ether (20 ml). Tranexamic acid (6.80 g), water (20 ml) and acetonitrile (15 ml) are added and the mixture is stirred for 10 minutes at 10-20° C. Triethylamine (6.2 ml) is charged over ˜5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for 10 minutes. After removal of aq. layer, the organic layer is washed with water (50 ml). The solvents are stripped off under reduced pressure with process temperature ≦35° C. and reactor jacket temperature ≦45° C. to yield the crude title product.

### Method E

To a jacketed laboratory reactor is charged 1-({[(3,4-methylenedioxyphenyl)oxy]carbonyl}oxy)ethyl 2-methylpropanoate (may be prepared as described in Description 2-13; 10.50 g) and tert-butyl methyl ether (20 ml). Tranexamic acid (7.30 g), water (20 ml) and acetonitrile (15 ml) are added and the mixture is stirred for 10 minutes at 10-20° C. Triethylamine (6.2 ml) is charged over ˜5 minutes. The reaction is stirred at 10-20° C. for 5 hrs. 10% potassium hydrogensulfate aqueous solution (67 ml) is charged to acidify reaction to pH 4-4.5. Tert-butyl methyl ether (80 ml) is charged and mixed for 10 minutes. After removal of aq. layer, the organic layer is washed with water (50 ml). The solvents are stripped off under reduced pressure with process temperature ≦35° C. and reactor jacket temperature ≦45° C. to yield the crude title product.

From the foregoing description, various modifications and changes in the compositions and methods provided herein will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

At least some of the chemical names of compounds of the present disclosure as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. and ChemDraw Ultra Version 10.0, available from CambridgeSoft®. In the instance where the indicated chemical name and the depicted structure differ, the depicted structure will control.

Chemical structures shown herein were prepared using ISIS®/DRAW or ChemDraw. Any open valency appearing on a carbon, oxygen or nitrogen atom in the structures herein indicates the presence of a hydrogen atom. Where a chiral center exists in a structure but no specific stereochemistry is shown for the chiral center, both enantiomers associated with the chiral structure are encompassed by the structure.

1. A method of making a compound of formula (I), or a stereoisomer thereof, a diastereomer thereof, or a salt of any one of foregoing, comprising:
(A) reacting a compound of formula (II), or a stereoisomer or a salt thereof, with R¹CO₂H to form a compound of formula (III); 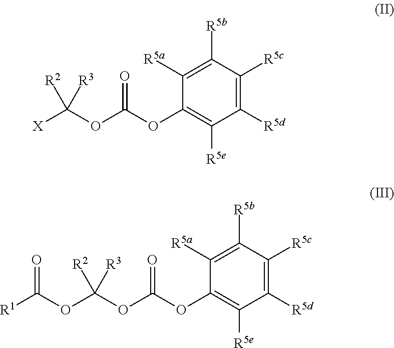
and
(B) reacting the compound of formula (III), or a stereoisomer or a salt thereof, with HNR^{4a}R^{4b} to form the compound of formula (I): 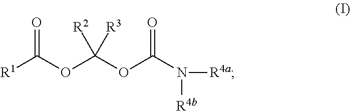
wherein:
each of R¹ and R² is independently C₁₋₄ alkyl;
R³ is H or C₁₋₄ alkyl;
HNR^{4a}R^{4b} is a drug molecule having an amino moiety;
R^{4a} and R^{4a} are groups of the drug molecule attached to the amino moiety;
each of R^{5a}, R^{5b}, R^{5c}, R^{5d}, and R^{5e} is independently selected from H, halo, C₁₋₄ alkyl, halo C₁₋₄ alkyl, phenyl, —C(O)O—C₁₋₄ alkyl, —C(O)—C₁₋₄ alkyl, —S(O)—C₁₋₄ alkyl, CN, —C(O)—NR^{6a}R^{6b}, substituted or unsubstituted C₁₋₄ alkoxy, and substituted or unsubstituted phenoxy; provided that at least one of R^{5a}, R^{5b}, R^{5c}, R^{5d}, and R^{5e} is other than H; or any two adjacent R^{5a}, R^{5b}, R^{5c}, R^{5d}, and R^{5e} are joined together to form a carbocycle or heterocycle;
each R^{6a} and R^{6b} is independently H, or C₁₋₄ alkyl; or R^{6a} and R^{6b} together with N they are attached to form heterocycle; and
X is a leaving group.

2. The method of claim 1, wherein X is halo.

3. The method of claim 1, wherein X is Cl.

4. The method of claim 1, wherein reaction step (A) occurs in a solvent.

5. The method of claim 4, wherein the solvent is selected from the group consisting of heptane, xylene, toluene, N-methylpyrrolidine, N,N-diisopropylamine, dimethyl formamide, dimethyl sulfoxide, diphenyl ether, and combinations thereof.

6. The method of claim 1, wherein reaction step (A) occurs at a temperature from about 50° C. to about 120° C.

7. The method of claim 1, wherein reaction step (A) occurs in the presence of a metal oxide.

8. The method of claim 1, wherein the reaction step (A) occurs in the presence of a metal alkanoate or a metal salt of R¹CO₂H.

9. The method of claim 1, wherein reaction step (A) occurs in the presence of R¹C(O)—O—C(O)R¹.

10. The method of claim 1, wherein reaction step (A) occurs in the presence of a tetraalkylammonium salt.

11. The method of claim 1, wherein reaction step (A) occurs in the presence of an organic base.

12. The method of claim 1, wherein reaction step (B) occurs in a solvent selected from the group consisting of heptane, xylene, toluene, dialkyl ether, cyclic ethers, dimethyl formamide, dimethyl sulfoxide, water, acetonitrile, ethyl acetate, and combinations thereof.

13. The method of claim 1, wherein reaction step (B) occurs at a temperature from about 0° C. to about 50° C.

14. The method of claim 1, wherein reaction step (B) occurs in the presence of a base.

15. The method of claim 1, wherein R¹ is i-Pr; R² is Me or i-Pr; and R³ is H.

16. The method of claim 1, wherein one or more of R^{5a}, R^{5b}, R^{5c}, R^{5d}, and R^{5e} is independently halo, C₁₋₄ alkyl, —C(O)O—C₁₋₄ alkyl, or substituted or unsubstituted alkoxy, and the rest are H.

17. The method of claim 1, wherein the drug molecule HNR^{4a}R^{4b} is selected from: acebutalol, albuterol, alprenolol, atenolol, bunolol, bupropion, butopamine, butoxamine, carbuterol, cartelolol, colterol, deterenol, dexpropanolol, diacetolol, dobutamine, exaprolol, exprenolol, fenoterol, fenyripol, labotolol, levobunolol, metolol, metaproterenol, metoprolol, nadolol, pamatolol, penbutalol, pindolol, pirbuterol, practolol, prenalterol, primidolol, prizidilol, procaterol, propanolol, quinterenol, rimiterol, ritodrine, solotol, soterenol, sulfiniolol, sulfinterol, sulictidil, tazaolol, terbutaline, timolol, tiprenolol, tipridil, tolamolol, thiabendazole, albendazole, albutoin, alendronate, alinidine, alizapride, amiloride, a minorex, aprinocid, cambendazole, cimetidine, cisapride, clonidine, cyclobenzadole, delavirdine, efegatrin, etintidine, fenbendazole, fenmetazole, flubendazole, fludorex, gabapentin, icadronate, lobendazole, mebendazole, metazoline, metoclopramide, methylphenidate, mexiletine, neridronate, nocodazole, oxfendazole, oxibendazole, oxmetidine, pamidronate, parbendazole, pramipexole, prazosin, pregabalin, procainamide, ranitidine, tetrahydrazoline, tiamenidine, tinazoline, tiotidine, tocamide, tolazoline, tramazoline, xylometazoline, dimethoxyphenethylamine, n-[3(R)-[2-piperidin-4-yl)ethyl]-2-piperidone-1-yl]acetyl-3(R)-methyl-β-alanine, adrenolone, aletamine, amidephrine, amphetamine, aspartame, bamethan, betahistine, carbidopa, clorprenaline, chlortermine, dopamine, L-dopa, ephrinephrine, etryptamine, fenfluramine, methyldopamine, norepinephrine, enviroxime, nifedipine, nimodipine, triamterene, pipedemic acid and similar compounds, 1-ethyl-6-fluoro-1,4-dihydro-4-oxo-7-(1-piperazinyl)-1,8-napthyridine-3-carboxylic acid and 1-cyclopropyl-6-fluoro-1,4-dihydro-4-oxo-7-(piperazinyl)-3-quinolinecarboxylic acid, theprubicin, deoxyspergualin, seglitide, nebracetam, benanomicin B, eremomycin, thrazarine, tosufloxacin, baogongteng A, angiopeptin, boholmycin, ravidomycin, tageflar, orienticins, amphotericin B, tiamdipine, doxorubicin, lysobactin, mofegiline, octreotide, oxolide, amikacin, phospholine, nuvanil, cispentacin, chlorotetain, remacemide, ramoplanins, janthinomycins, mersacidin, droxidopa, helvecardin A, helvecardin B, rilmazafone, vigabatrin, amlodipine, (R)-(+)-amlodipine, mideplanin, milnacipran, pranedipine, olradipine, deoxymethylspergualin, fudosteine, trovafloxacin, ceranapril, restricticin, idarubicin, arbekacin, giracodazole, poststatin, pazufloxacin, D-cycloserine, ovothiol A, ceftizoxime, icatibant, p-iodorubidazone, aladapcin, dalargin, seproxetine, pradimicin E, pradimicin FA-2, tafenoquine, sampatrilat, ruboxyl, dactimicin, alatrofloxacin, galarubicin, metaraminol, exatecan, squalamine, paromomycin, leustroducsin A, leustroducsin B, leustroducsin C, lanicemine, azoxybacilin, tetrafibricin, pixantrone, ziconotide, garomefrine, spinorphin, doripenem, alestramustine, seraspenide, safingol, aminolevulinic acid, pelagiomicin C, styloguanidine, L-4-oxalysine, eglumegad, rhodopeptins, mycestericin E, midaxifylline, anisperimus, lagatide, ibutamoren, oritavancin, ecenofloxacin, metyrosine, methyldopa, baclofen, tranylcypromine, micronomicin, zorubicin, epirubicin, gilatide, epithalon, cystamine, pluraflavin A, pluraflavin B, pasireotide, caprazamycin, barusiban, spisulosine, 21-aminoepothilone B, capsavanil, olcegepant, sulphostin, lobophorin A, papuamide A, papuamide B, cystocin, deoxynegamycin, galnon, pyloricidin B, brasilicardin A, neramexane, kaitocephalin, icofungipen, aliskiren, capromorelin, histaprodifen, donitriptan, cambrescidins, tipifarnib, tabimorelin, belactosin A, belactosin C, circinamide, targinine, sulphazocine, nepicastat, oseltamivir, hydrostatin A, butabindide, netamiftide, memantine, fluvoxamine, deferoxamine, tranexamic acid, fortimicin A, cefaclor, lisinopril, ubestatin, cefminox, aspoxicillin, cefcanel, cefcanel daloxate, olamufloxacin, R-(+)-aminoindane, gemifloxacin, kahalalide F, palau'amine, examorelin, leustroducsin H, sabarubicin, amifostine, L-homothiocitrulline, L-thiocitrulline, impentamine, neboglamine, amselamine, cetefloxacin, cyclothialidine, fluvirucin B2, loracarbef, cefprozil, sperabillins, milacamide, avizafone, α-methyltryptophan, cytaramycin, lanomycin, decaplanin, eflornithine, L-histidinol, tuftsin, kanamycin, amthamine, sitafloxacin, leurubicin, amantadine, isodoxorubicin, gludopa, bactobolin, esafloxacin, tabilautide, lazabemide, enalkiren, amrubicin, daunorubicin, mureidomycins, pyridazomycin, cimaterol, (+)-isamoltan, N-desmethylmilameline, noberastine, fosopamine, adaprolol, pradimicin B, amosulalol, xamoterol, boholmycin, risotilide, indeloxazine, denopamine, parodilol, utibapril, nardeterol, biemnidin, sparfloxacin, sibanomicin, tianeptine, oberadilol, methoctramine, sezolamide, anabasine, zilpaterol, zabiciprilat, enkastins, ulifloxacin, (+)-sotalol, deoxynojirimycin, altromycin A, altromycin C, dorzolamide, fepradinol, delapril, ciprofloxacin, balofloxacin, mepindolol, berlafenone, ramipril, dopexamine, dilevalol, (−)-nebivolol, duramycin, enalapril, meluadrine, zelandopam, voglibose, sertraline, carvedilol, pafenolol, paroxetine, fluoxetine, phendioxan, salmeterol, solpecainol, repinotan, bambuterol, safinamide, tilisolol, 7-oxostaurosporine, caldaret, sertraline, cilazapril, benazepril, prisotinol, gatifloxacin, ovothiol B, adaprolol, tienoxolol, fluparoxan, alprenoxime, efegatran, pradimicin, salbostatin, ersentilide, (S)-noremopamil, esperamicin A1, batoprazine, ersentilide, osutidine, quinapril, dihydrexidine, argiopine, pradimicin D, frovatriptan, hispidospermidin, silodosin, michellamine B, sibenadet, tetrindol, talibegron, topixantrone, nortopixantrone, tecalcet, buteranol, α-methylepinephrine, nornicotine, thiofedrine, lenapenem, imidapril, epibatidine, premafloxacin, socorromycin, trandolapril, tamsulosin, dirithromycin, inogatran, vicenistatin, immepyr, immepip, balanol, orbifloxacin, maropitant, dabelotine, lerisetron, ertapenem, nolomirole, moxifloxacin, vofopitant, halofuginone, melagatran, ximelagatran, fasudil, isofagomine, pseudoephedrine, propafenone, celiprolol, carteolol, penbutolol, labetalol, acebutolol, reproterol, rimoterol, amoxapine, maprotiline, viloxazine, protriptyline, nortriptyline, desipramine, oxprenolol, propranolol, ketamine, butofilolol, flecamide, tulobuterol, befunolol, immucillin-H, vestipitant, cinacalcet, lapatinib, desloratadine, ladostigil, vildagliptin, tulathromycin B, becampanel, salbutamol, delucemine, solabegron, paroxetine, gaboxadol, telavancin, ralfinamide, tomoxetine, dalbavancin, elarofiban, ferulinolol, fenoldopam, sumanirole, sarizotan, brinzolamide, pradofloxacin, garenoxacin, reboxetine, ezlopitant, palindore, nebivolol, dinapsoline, proxodolol, repinotan, demexiptiline, mitoxantrone, norfloxacin, dilevalol, nipradilol, esmolol, ibopamine, troxipide, arotinolol, formoterol, bopindolol, cloranolol, mefloquine, perindopril, mabuterol, bisoprolol, bevantolol, betaxolol, tertatolol, enoxacin, lotrafiban, moexipril, droxinavir, adrogolide, alniditan, tigecycline, lubazodone, meropenem, temocapril, napsamycins, (−)-cicloprolol, ecteinascidins, alprafenone, landiolol, tirofiban, noberastine, rasagiline, setazindol, picumeterol, arbutamine, mecamylamine, delfaprazine, imidapril, midafotel, manzamines, binospirone, duloxetine, and litoxetine.

18. The method of claim 1, wherein R^{4a} is H; and R^{4b} is selected from

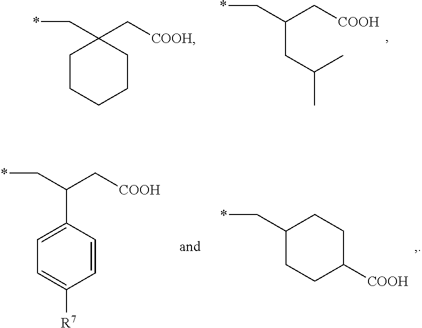

19. The method of claim 18, wherein R⁷ is Cl or F.

20. The method of claim 1, wherein the compound of formula (I) is a compound according to formula (IVa), (IVb), (IVc), (IVd), or (IVe): 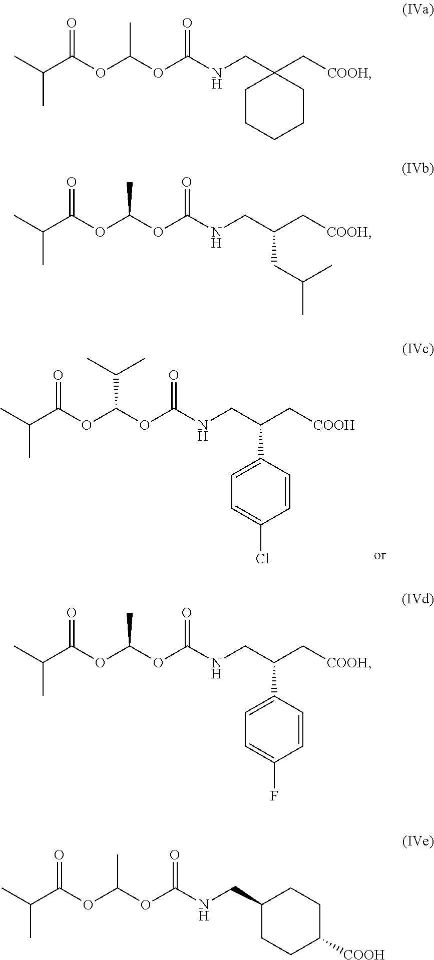 [SMILES: CC(OC(=O)NCC1(CC(=O)O)CCCCC1)OC(=O)C(C)C ; CC(C)C[C@H](CNC(=O)O[C@H](C)OC(=O)C(C)C)CC(=O)O ; CC(C)C(=O)O[C@@H](OC(=O)NC[C@H](CC(=O)O)c1ccc(Cl)cc1)C(C)C ; CC(C)C(=O)O[C@@H](C)OC(=O)NC[C@H](CC(=O)O)c1ccc(F)cc1 ; CC(OC(=O)NC[C@H]1CC[C@H](C(=O)O)CC1)OC(=O)C(C)C]
or a salt thereof.

21. A compound according to formula (III): 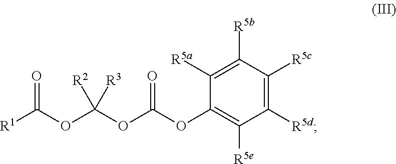
a stereoisomer thereof, a diastereomer thereof; or a salt of any one of foregoing;
wherein
each of R¹ and R² is independently C₁₋₄ alkyl; R³ is H or C₁₋₄ alkyl;
each of R^{5a}, R^{5b}, R^{5c}, R^{5d}, or R^{5e} is independently selected from a group consisting of H, halo, C₁₋₄ alkyl, halo C₁₋₄ alkyl, phenyl, —C(O)O—C₁₋₄ alkyl, —C(O)—C₁₋₄ alkyl, —S(O)—C₁₋₄ alkyl, CN, —C(O)—NR^{6a}R^{6b}, substituted or unsubstituted C₁₋₄ alkoxy, and substituted or unsubstituted phenoxy; and
each of R^{6a} and R^{6b} is independently H, or C₁₋₄ alkyl; or R^{6a} and R^{6b} together with N they are attached to form heterocycle;
provided that at least one of R^{5a}, R^{5b}, R^{5c}, R^{5d}, and R^{5e} is other than H; or any two of R^{5a}, R^{5b}, R^{5c}, R^{5d}, and R^{5e} form O—CH₂—O—, or O—CH₂—CH₂—O—.

22. The compound of claim 21, wherein R¹ is i-Pr; R² is methyl or i-Pr; and R³ is H.

23. The compound of claim 21, wherein one or more of R^{5a}, R^{5b}, R^{5c}, R^{5d}, and R^{5e} is halo, and the rest are H.

24. The compound of claim 21, wherein one of R^{5a}, R^{5b}, R^{5c}, R^{5d}, and R^{5e} is Me, Et, —C(O)OMe, —C(O)OEt, —OMe, —CF₃, CN, —C(O)Me, —S(O)Me, —C(O)NH₂, —C(O)NMe₂, and the rest are H.

25. The compound of claim 21, wherein the compound is a compound according to formula (VIIa), (VIIb), (VIIc), (VIId), (VIIe), (VIIf), (VIIg), or (VIIh): 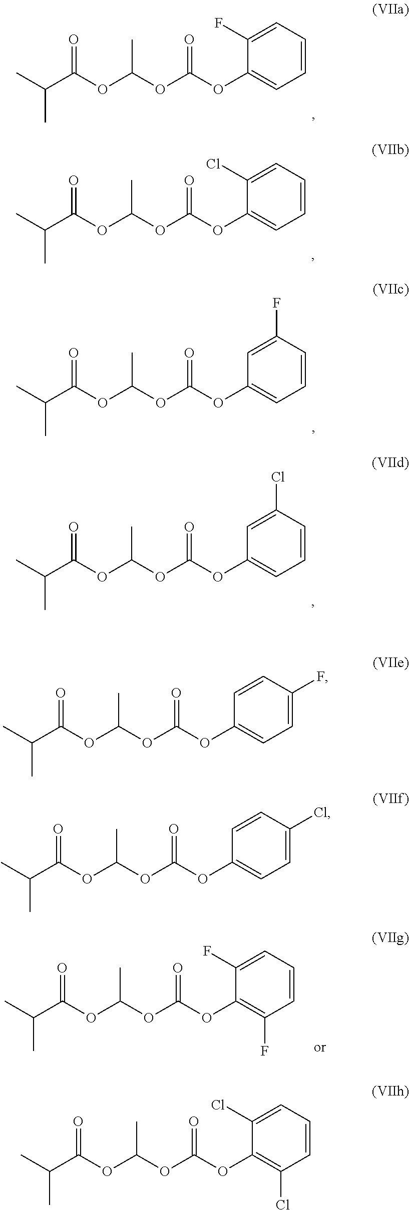
a stereoisomer thereof, a diastereomer thereof; or a salt of any one of foregoing.

26. The compound of claim 21, wherein the compound is a compound according to formula (VIIIa), (VIIIb), (VIIIc), (VIIId), (VIIIe), or (VIIIf): 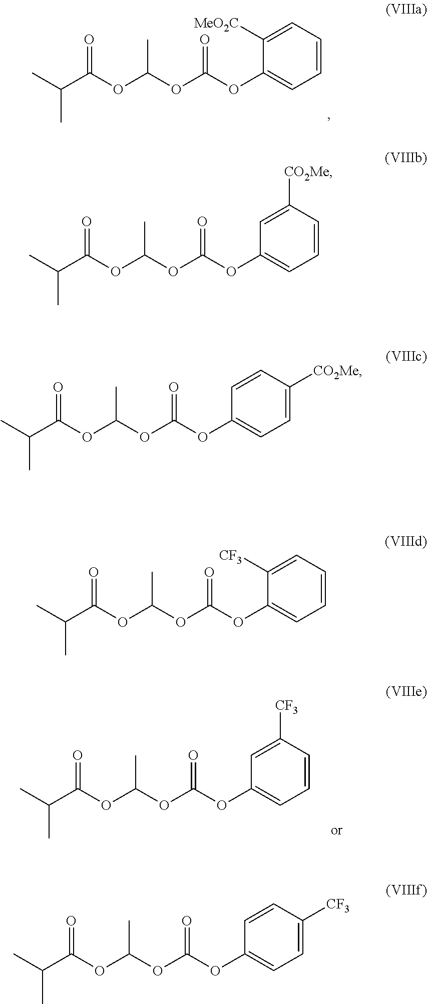 [SMILES: COC(=O)c1ccccc1OC(=O)OC(C)OC(=O)C(C)C ; COC(=O)c1cccc(OC(=O)OC(C)OC(=O)C(C)C)c1 ; COC(=O)c1ccc(OC(=O)OC(C)OC(=O)C(C)C)cc1 ; CC(OC(=O)Oc1ccccc1C(F)(F)F)OC(=O)C(C)C ; CC(OC(=O)Oc1cccc(C(F)(F)F)c1)OC(=O)C(C)C ; CC(OC(=O)Oc1ccc(C(F)(F)F)cc1)OC(=O)C(C)C]
a stereoisomer thereof, a diastereomer thereof; or a salt of any one of foregoing.

27. The compound of claim 21, wherein the compound is a compound according to formula (IXa), (IXb), (IXc), (IXd), (IXe), (IXf), (IXg), or (IXh): 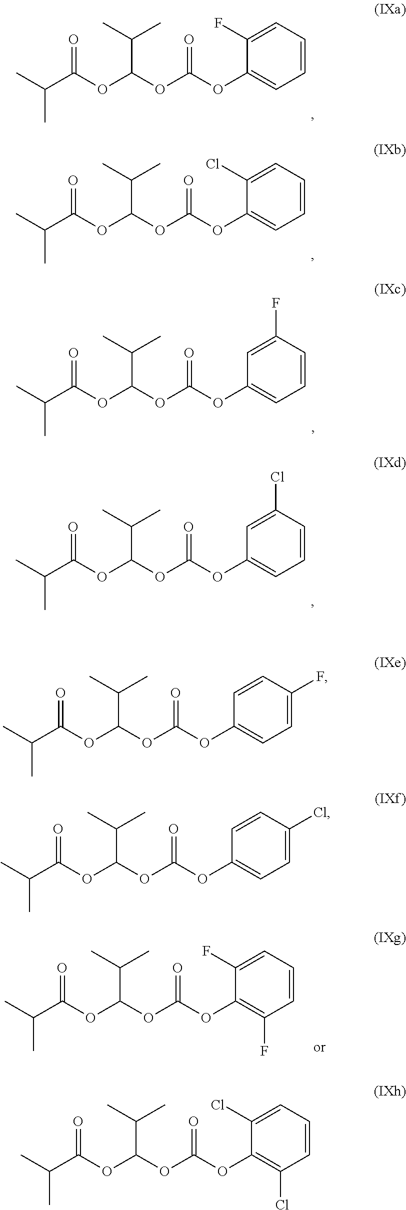
a stereoisomer thereof, a diastereomer thereof; or a salt of any one of foregoing.

28. The compound of claim 21, wherein the compound is a compound according to formula (Xa), (Xb), (Xc), (Xd), (Xe), or (Xf): 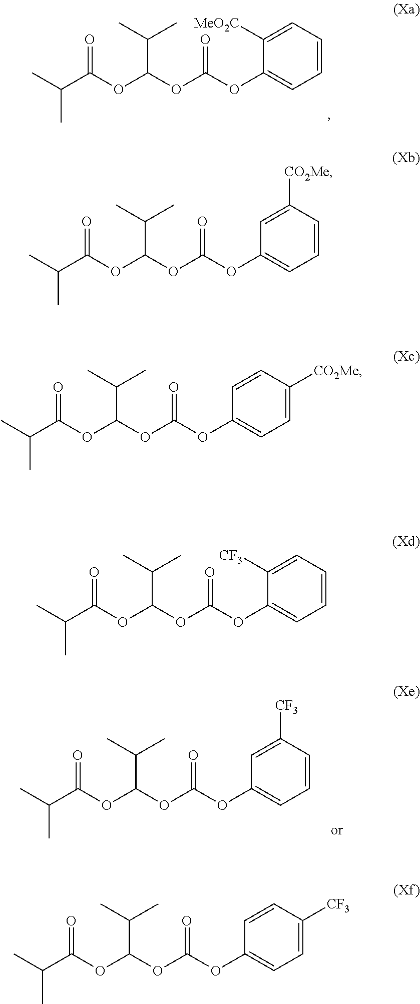 [SMILES: COC(=O)c1ccccc1OC(=O)OC(OC(=O)C(C)C)C(C)C ; COC(=O)c1cccc(OC(=O)OC(OC(=O)C(C)C)C(C)C)c1 ; COC(=O)c1ccc(OC(=O)OC(OC(=O)C(C)C)C(C)C)cc1 ; CC(C)C(=O)OC(OC(=O)Oc1ccccc1C(F)(F)F)C(C)C ; CC(C)C(=O)OC(OC(=O)Oc1cccc(C(F)(F)F)c1)C(C)C ; CC(C)C(=O)OC(OC(=O)Oc1ccc(C(F)(F)F)cc1)C(C)C]
a stereoisomer thereof, a diastereomer thereof; or a salt of any one of foregoing.

29. The compound of claim 21, wherein the compound is a compound according to formula (XIa), (XIb), (XIc), (XId), (XIe), (XIf), (XIg), or (XIh): 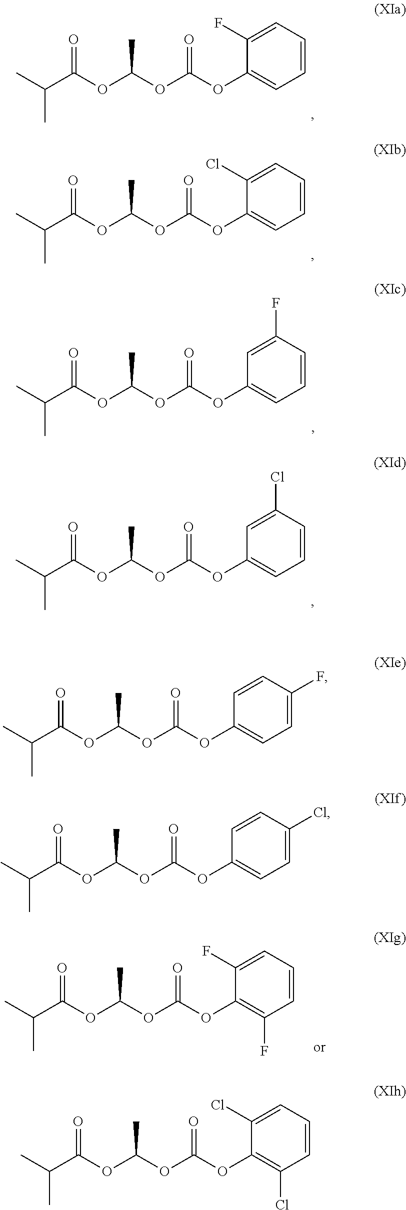
a stereoisomer thereof, a diastereomer thereof; or a salt of any of the foregoing.

30. The compound of claim 21, wherein the compound is a compound according to formula (XIIa), (XIIb), (XIIc), (XIId), (XIIe), or (XIIf): 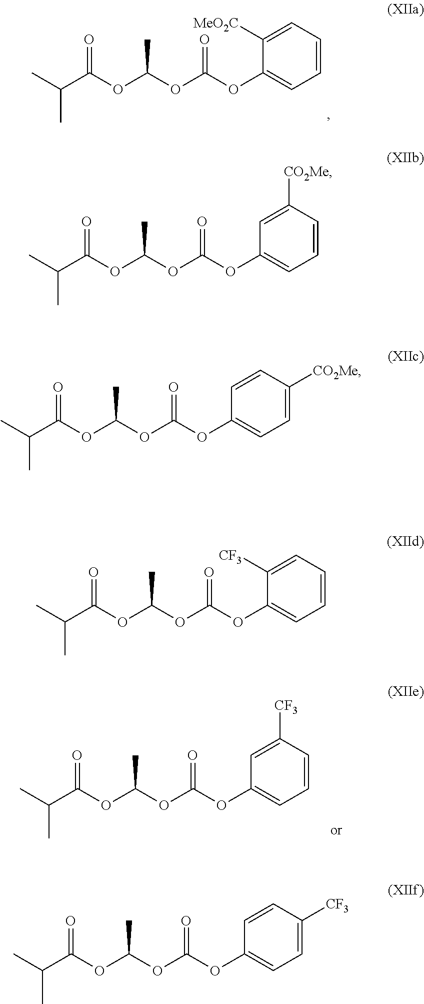 [SMILES: COC(=O)c1ccccc1OC(=O)O[C@H](C)OC(=O)C(C)C ; COC(=O)c1cccc(OC(=O)O[C@H](C)OC(=O)C(C)C)c1 ; COC(=O)c1ccc(OC(=O)O[C@H](C)OC(=O)C(C)C)cc1 ; CC(C)C(=O)O[C@@H](C)OC(=O)Oc1ccccc1C(F)(F)F ; CC(C)C(=O)O[C@@H](C)OC(=O)Oc1cccc(C(F)(F)F)c1 ; CC(C)C(=O)O[C@@H](C)OC(=O)Oc1ccc(C(F)(F)F)cc1]
a stereoisomer thereof, a diastereomer thereof; or a salt thereof.

31. The compound of claim 21, wherein the compound is a compound according to formula (XIIIa), (XIIIb), (XIIIc), (XIIId), (XIIIe), (XIIIf), (XIIIg), or (XIIIh) 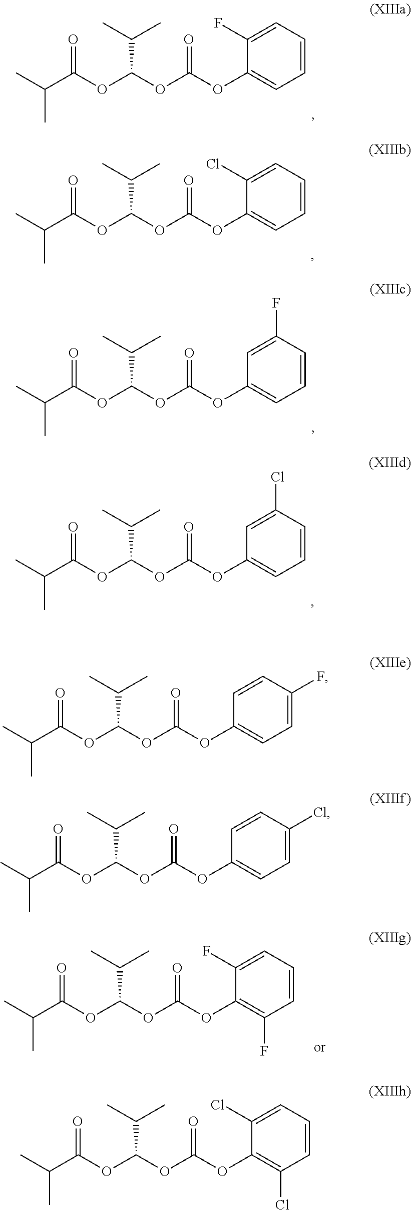
a stereoisomer thereof, a diastereomer thereof; or a salt of any of the foregoing.

32. The compound of claim 21, wherein the compound is a compound according to formula (XIVa), (XIVb), (XIVc), (XIVd), (XIVe), or (XIVf): 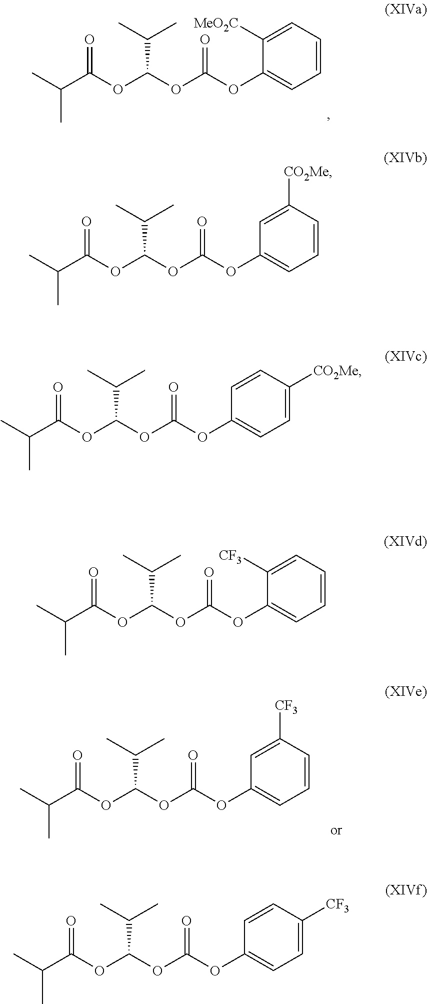 [SMILES: COC(=O)c1ccccc1OC(=O)O[C@H](OC(=O)C(C)C)C(C)C ; COC(=O)c1cccc(OC(=O)O[C@H](OC(=O)C(C)C)C(C)C)c1 ; COC(=O)c1ccc(OC(=O)O[C@H](OC(=O)C(C)C)C(C)C)cc1 ; CC(C)C(=O)O[C@@H](OC(=O)Oc1ccccc1C(F)(F)F)C(C)C ; CC(C)C(=O)O[C@@H](OC(=O)Oc1cccc(C(F)(F)F)c1)C(C)C ; CC(C)C(=O)O[C@@H](OC(=O)Oc1ccc(C(F)(F)F)cc1)C(C)C]
a stereoisomer thereof, a diastereomer thereof; or a salt of any of the foregoing.

33. The compound of claim 21, wherein the compound is a compound according to formula (XVa), (XVb), (XVc), (XVd), (XVe), or (XVf): 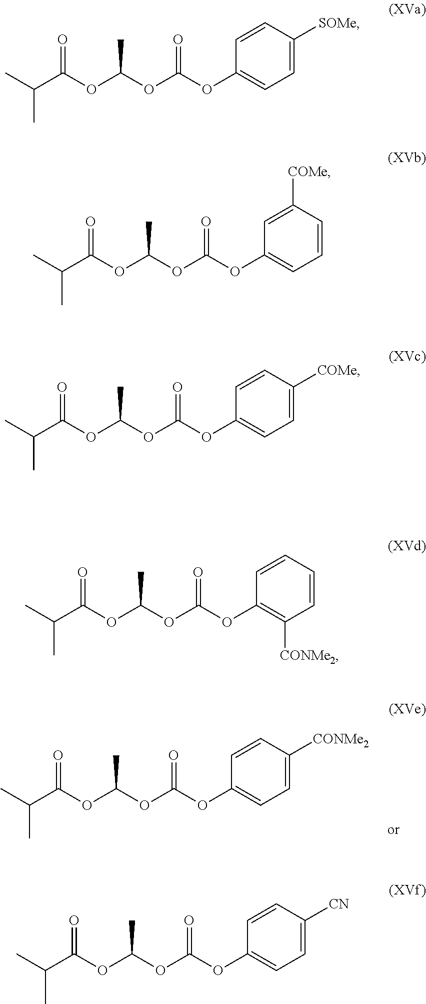
a stereoisomer thereof, a diastereomer thereof; or a salt thereof.

34. The compound of claim 21, wherein the compound is a compound according to formula (XVIa), (XVIb), (XVIc), (XVId), (XVIe), or (XVIf): 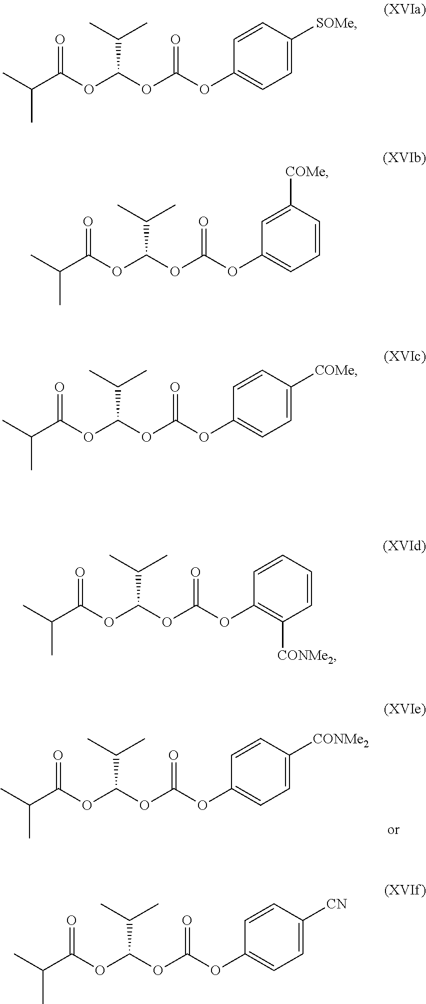
a stereoisomer thereof, a diastereomer thereof; or a salt thereof.
